SEQUENCE LISTING (245 sequences)

SEQ ID NO: 1 - protein, 8 aa, Artificial Sequence
  Description of Artificial Sequence Synthetic peptide
QWAVGHXL

SEQ ID NO: 2 - protein, 8 aa, Artificial Sequence
  Description of Artificial Sequence Synthetic peptide
QWAVGHXL

SEQ ID NO: 3 - protein, 8 aa, Artificial Sequence
  Description of Artificial Sequence Synthetic peptide
QWAVGHXL

SEQ ID NO: 4 - protein, 8 aa, Artificial Sequence
  Description of Artificial Sequence Synthetic peptide
QWAVGHXL

SEQ ID NO: 5 - protein, 8 aa, Artificial Sequence
  Description of Artificial Sequence Synthetic peptide
QWAVGHXX

SEQ ID NO: 6 - protein, 4 aa, Artificial Sequence
  Description of Artificial Sequence Synthetic peptide
VAFG

SEQ ID NO: 7 - protein, 8 aa, Artificial Sequence
  Description of Artificial Sequence Synthetic peptide
QWAVGHXX

SEQ ID NO: 8 - protein, 8 aa, Artificial Sequence
  Description of Artificial Sequence Synthetic peptide
QWAVGHXL

SEQ ID NO: 9 - protein, 4 aa, Artificial Sequence
  Description of Artificial Sequence Synthetic peptide
VAHG

SEQ ID NO: 10 - protein, 10 aa, Artificial Sequence
  Description of Artificial Sequence Synthetic peptide
RXQWAVGHXL

SEQ ID NO: 11 - protein, 4 aa, Artificial Sequence
  Description of Artificial Sequence Synthetic peptide
VAFG

SEQ ID NO: 12 - protein, 8 aa, Artificial Sequence
  Description of Artificial Sequence Synthetic peptide
QWAVGHXL

SEQ ID NO: 13 - protein, 4 aa, Artificial Sequence
  Description of Artificial Sequence Synthetic peptide
VAHG

SEQ ID NO: 14 - protein, 9 aa, Artificial Sequence
  Description of Artificial Sequence Synthetic peptide
XQWAVGHXL

SEQ ID NO: 15 - protein, 4 aa, Artificial Sequence
  Description of Artificial Sequence Synthetic peptide
VAFG

SEQ ID NO: 16 - protein, 10 aa, Artificial Sequence
  Description of Artificial Sequence Synthetic peptide
RXQWAVGHXL

SEQ ID NO: 17 - protein, 8 aa, Artificial Sequence
  Description of Artificial Sequence Synthetic peptide
QWAVGHXL

SEQ ID NO: 18 - protein, 9 aa, Artificial Sequence
  Description of Artificial Sequence Synthetic peptide
XQWAVGHXL

SEQ ID NO: 19 - protein, 8 aa, Artificial Sequence
  Description of Artificial Sequence Synthetic peptide
QWAVGHXL

SEQ ID NO: 20 - protein, 9 aa, Artificial Sequence
  Description of Artificial Sequence Synthetic peptide
XQWAVGHXL

SEQ ID NO: 21 - protein, 9 aa, Artificial Sequence
  Description of Artificial Sequence Synthetic peptide
XQWAVGHXX

SEQ ID NO: 22 - protein, 9 aa, Artificial Sequence
  Description of Artificial Sequence Synthetic peptide
XQWAVGHXL

SEQ ID NO: 23 - protein, 8 aa, Artificial Sequence
  Description of Artificial Sequence Synthetic peptide
QWAVGHXX

SEQ ID NO: 24 - protein, 9 aa, Artificial Sequence
  Description of Artificial Sequence Synthetic peptide
XQWAVGHXL

SEQ ID NO: 25 - protein, 10 aa, Artificial Sequence
  Description of Artificial Sequence Synthetic peptide
RXQWAVGHXL

SEQ ID NO: 26 - protein, 10 aa, Artificial Sequence
  Description of Artificial Sequence Synthetic peptide
RXQWAVGHXL

SEQ ID NO: 27 - protein, 8 aa, Artificial Sequence
  Description of Artificial Sequence Synthetic peptide
QWAVGHXX

SEQ ID NO: 28 - protein, 8 aa, Artificial Sequence
  Description of Artificial Sequence Synthetic peptide
QWAVGHXG

SEQ ID NO: 29 - protein, 9 aa, Artificial Sequence
  Description of Artificial Sequence Synthetic peptide
XQWAVGHXL

SEQ ID NO: 30 - protein, 8 aa, Artificial Sequence
  Description of Artificial Sequence Synthetic peptide
QWAVGHXG

SEQ ID NO: 31 - protein, 10 aa, Artificial Sequence
  Description of Artificial Sequence Synthetic peptide
RXQWAVGHXL

SEQ ID NO: 32 - protein, 8 aa, Artificial Sequence
  Description of Artificial Sequence Synthetic peptide
QWAVGHXL

SEQ ID NO: 33 - protein, 8 aa, Artificial Sequence
  Description of Artificial Sequence Synthetic peptide
QWAVGHXG

SEQ ID NO: 34 - protein, 8 aa, Artificial Sequence
  Description of Artificial Sequence Synthetic peptide
QWAVGHXX

SEQ ID NO: 35 - protein, 8 aa, Artificial Sequence
  Description of Artificial Sequence Synthetic peptide
QWAVGHXX

SEQ ID NO: 36 - protein, 8 aa, Artificial Sequence
  Description of Artificial Sequence Synthetic peptide
QWAVGHXA

SEQ ID NO: 37 - protein, 10 aa, Artificial Sequence
  Description of Artificial Sequence Synthetic peptide
RXQWAVGHXL

SEQ ID NO: 38 - protein, 10 aa, Artificial Sequence
  Description of Artificial Sequence Synthetic peptide
RXQWAVGHXL

SEQ ID NO: 39 - protein, 9 aa, Artificial Sequence
  Description of Artificial Sequence Synthetic peptide
XQWAVGHXL

SEQ ID NO: 40 - protein, 8 aa, Artificial Sequence
  Description of Artificial Sequence Synthetic peptide
QWAVGHXL

SEQ ID NO: 41 - protein, 9 aa, Artificial Sequence
  Description of Artificial Sequence Synthetic peptide
XQWAVGHXL

SEQ ID NO: 42 - protein, 8 aa, Artificial Sequence
  Description of Artificial Sequence Synthetic peptide
QWAVGHXX

SEQ ID NO: 43 - protein, 8 aa, Artificial Sequence
  Description of Artificial Sequence Synthetic peptide
QWAVGHXG

SEQ ID NO: 44 - protein, 9 aa, Artificial Sequence
  Description of Artificial Sequence Synthetic peptide
XQWAVGHXX

SEQ ID NO: 45 - protein, 9 aa, Artificial Sequence
  Description of Artificial Sequence Synthetic peptide
XQWAVGHXL

SEQ ID NO: 46 - protein, 8 aa, Artificial Sequence
  Description of Artificial Sequence Synthetic peptide
QWAVGHXL

SEQ ID NO: 47 - protein, 9 aa, Artificial Sequence
  Description of Artificial Sequence Synthetic peptide
XQWAVGHXL

SEQ ID NO: 48 - protein, 8 aa, Artificial Sequence
  Description of Artificial Sequence Synthetic peptide
QWAVGHXL

SEQ ID NO: 49 - protein, 8 aa, Artificial Sequence
  Description of Artificial Sequence Synthetic peptide
QWAVGHXL

SEQ ID NO: 50 - protein, 8 aa, Artificial Sequence
  Description of Artificial Sequence Synthetic peptide
QWAVGHXL

SEQ ID NO: 51 - protein, 8 aa, Artificial Sequence
  Description of Artificial Sequence Synthetic peptide
QWAVGHXL

SEQ ID NO: 52 - protein, 8 aa, Artificial Sequence
  Description of Artificial Sequence Synthetic peptide
QWAVAHXX

SEQ ID NO: 53 - protein, 8 aa, Artificial Sequence
  Description of Artificial Sequence Synthetic peptide
QWAVAHFX

SEQ ID NO: 54 - protein, 8 aa, Artificial Sequence
  Description of Artificial Sequence Synthetic peptide
QWAVAHFL

SEQ ID NO: 55 - protein, 8 aa, Artificial Sequence
  Description of Artificial Sequence Synthetic peptide
QWAVAHFL

SEQ ID NO: 56 - protein, 8 aa, Artificial Sequence
  Description of Artificial Sequence Synthetic peptide
QWAVAHLL

SEQ ID NO: 57 - protein, 8 aa, Artificial Sequence
  Description of Artificial Sequence Synthetic peptide
QWAVAHIL

SEQ ID NO: 58 - protein, 8 aa, Artificial Sequence
  Description of Artificial Sequence Synthetic peptide
QWAVAHLG

SEQ ID NO: 59 - protein, 8 aa, Artificial Sequence
  Description of Artificial Sequence Synthetic peptide
QWAVAHFX

SEQ ID NO: 60 - protein, 8 aa, Artificial Sequence
  Description of Artificial Sequence Synthetic peptide
QWAVAHFX

SEQ ID NO: 61 - protein, 8 aa, Artificial Sequence
  Description of Artificial Sequence Synthetic peptide
QWAVAHFG

SEQ ID NO: 62 - protein, 8 aa, Artificial Sequence
  Description of Artificial Sequence Synthetic peptide
QWAVAHXG

SEQ ID NO: 63 - protein, 8 aa, Artificial Sequence
  Description of Artificial Sequence Synthetic peptide
QWAVAHXG

SEQ ID NO: 64 - protein, 8 aa, Artificial Sequence
  Description of Artificial Sequence Synthetic peptide
QWAVAHFX

SEQ ID NO: 65 - protein, 8 aa, Artificial Sequence
  Description of Artificial Sequence Synthetic peptide
QWAVAHFL

SEQ ID NO: 66 - protein, 8 aa, Artificial Sequence
  Description of Artificial Sequence Synthetic peptide
QWAVAHFL

SEQ ID NO: 67 - protein, 8 aa, Artificial Sequence
  Description of Artificial Sequence Synthetic peptide
QWAVAHFL

SEQ ID NO: 68 - protein, 8 aa, Artificial Sequence
  Description of Artificial Sequence Synthetic peptide
QWAVAHFL

SEQ ID NO: 69 - protein, 8 aa, Artificial Sequence
  Description of Artificial Sequence Synthetic peptide
QWAVAHFL

SEQ ID NO: 70 - protein, 8 aa, Artificial Sequence
  Description of Artificial Sequence Synthetic peptide
QWAVAHFL

SEQ ID NO: 71 - protein, 8 aa, Artificial Sequence
  Description of Artificial Sequence Synthetic peptide
QWAVAHIG

SEQ ID NO: 72 - protein, 8 aa, Artificial Sequence
  Description of Artificial Sequence Synthetic peptide
QWAVGHXX

SEQ ID NO: 73 - protein, 8 aa, Artificial Sequence
  Description of Artificial Sequence Synthetic peptide
QWAVGHXX

SEQ ID NO: 74 - protein, 8 aa, Artificial Sequence
  Description of Artificial Sequence Synthetic peptide
QWAVGHXA

SEQ ID NO: 75 - protein, 8 aa, Artificial Sequence
  Description of Artificial Sequence Synthetic peptide
QWAVGHXA

SEQ ID NO: 76 - protein, 10 aa, Artificial Sequence
  Description of Artificial Sequence Synthetic peptide
KAQWAVGHXL

SEQ ID NO: 77 - protein, 7 aa, Artificial Sequence
  Description of Artificial Sequence Synthetic peptide
QWAVHXL

SEQ ID NO: 78 - protein, 10 aa, Artificial Sequence
  Description of Artificial Sequence Synthetic peptide
KAQWAVGHXL

SEQ ID NO: 79 - protein, 10 aa, Artificial Sequence
  Description of Artificial Sequence Synthetic peptide
RSQWAVGHXL

SEQ ID NO: 80 - protein, 10 aa, Artificial Sequence
  Description of Artificial Sequence Synthetic peptide
SSQWAVGHXL

SEQ ID NO: 81 - protein, 10 aa, Artificial Sequence
  Description of Artificial Sequence Synthetic peptide
KSQWAVGHXL

SEQ ID NO: 82 - protein, 8 aa, Artificial Sequence
  Description of Artificial Sequence Synthetic peptide
QWAVGHXL

SEQ ID NO: 83 - protein, 10 aa, Artificial Sequence
  Description of Artificial Sequence Synthetic peptide
RSQWAVGHXL

SEQ ID NO: 84 - protein, 10 aa, Artificial Sequence
  Description of Artificial Sequence Synthetic peptide
KAQWAVGHXL

SEQ ID NO: 85 - protein, 9 aa, Artificial Sequence
  Description of Artificial Sequence Synthetic peptide
XQWAVGHXL

SEQ ID NO: 86 - protein, 10 aa, Artificial Sequence
  Description of Artificial Sequence Synthetic peptide
RXQWAVGHXL

SEQ ID NO: 87 - protein, 10 aa, Artificial Sequence
  Description of Artificial Sequence Synthetic peptide
RXQWAVGHXL

SEQ ID NO: 88 - protein, 9 aa, Artificial Sequence
  Description of Artificial Sequence Synthetic peptide
XQWAVGHXL

SEQ ID NO: 89 - protein, 8 aa, Artificial Sequence
  Description of Artificial Sequence Synthetic peptide
QWAVGHXL

SEQ ID NO: 90 - protein, 8 aa, Artificial Sequence
  Description of Artificial Sequence Synthetic peptide
QWAVGHXL

SEQ ID NO: 91 - protein, 8 aa, Artificial Sequence
  Description of Artificial Sequence Synthetic peptide
QWAVGHXL

SEQ ID NO: 92 - protein, 8 aa, Artificial Sequence
  Description of Artificial Sequence Synthetic peptide
QWAVGHXL

SEQ ID NO: 93 - protein, 10 aa, Artificial Sequence
  Description of Artificial Sequence Synthetic peptide
RXQWAVGHXL

SEQ ID NO: 94 - protein, 9 aa, Artificial Sequence
  Description of Artificial Sequence Synthetic peptide
XQWAVGHXX

SEQ ID NO: 95 - protein, 10 aa, Artificial Sequence
  Description of Artificial Sequence Synthetic peptide
SSQWAVGHXL

SEQ ID NO: 96 - protein, 9 aa, Artificial Sequence
  Description of Artificial Sequence Synthetic peptide
XQWAVGHXL

SEQ ID NO: 97 - protein, 9 aa, Artificial Sequence
  Description of Artificial Sequence Synthetic peptide
XQWAVGHXL

SEQ ID NO: 98 - protein, 9 aa, Artificial Sequence
  Description of Artificial Sequence Synthetic peptide
XQWAVGHXX

SEQ ID NO: 99 - protein, 9 aa, Artificial Sequence
  Description of Artificial Sequence Synthetic peptide
XQWAVGHXG

SEQ ID NO: 100 - protein, 9 aa, Artificial Sequence
  Description of Artificial Sequence Synthetic peptide
XQWAVGHXL

SEQ ID NO: 101 - protein, 9 aa, Artificial Sequence
  Description of Artificial Sequence Synthetic peptide
QWAVGHXXL

SEQ ID NO: 102 - protein, 9 aa, Artificial Sequence
  Description of Artificial Sequence Synthetic peptide
QWAVGHXXX

SEQ ID NO: 103 - protein, 9 aa, Artificial Sequence
  Description of Artificial Sequence Synthetic peptide
XQWAVGHXG

SEQ ID NO: 104 - protein, 6 aa, Artificial Sequence
  Description of Artificial Sequence Synthetic peptide
KFXWKT

SEQ ID NO: 105 - protein, 6 aa, Artificial Sequence
  Description of Artificial Sequence Synthetic peptide
XMFYWK

SEQ ID NO: 106 - protein, 8 aa, Artificial Sequence
  Description of Artificial Sequence Synthetic peptide
CLITRCRY

SEQ ID NO: 107 - protein, 8 aa, Artificial Sequence
  Description of Artificial Sequence Synthetic peptide
CLIVRCRY

SEQ ID NO: 108 - protein, 9 aa, Artificial Sequence
  Description of Artificial Sequence Synthetic peptide
XQWAVGHXL

SEQ ID NO: 109 - protein, 9 aa, Artificial Sequence
  Description of Artificial Sequence Synthetic peptide
XQWAVGHXL

SEQ ID NO: 110 - protein, 9 aa, Artificial Sequence
  Description of Artificial Sequence Synthetic peptide
XQWAVGHXX

SEQ ID NO: 111 - protein, 10 aa, Artificial Sequence
  Description of Artificial Sequence Synthetic peptide
RXQWAVGHXL

SEQ ID NO: 112 - protein, 8 aa, Artificial Sequence
  Description of Artificial Sequence Synthetic peptide
QWAVGHXL

SEQ ID NO: 113 - protein, 10 aa, Artificial Sequence
  Description of Artificial Sequence Synthetic peptide
RXQWAVGHXL

SEQ ID NO: 114 - protein, 10 aa, Artificial Sequence
  Description of Artificial Sequence Synthetic peptide
RXQWAVGHXL

SEQ ID NO: 115 - protein, 11 aa, Artificial Sequence
  Description of Artificial Sequence Synthetic peptide
RARQWAVGHXL

SEQ ID NO: 116 - protein, 9 aa, Artificial Sequence
  Description of Artificial Sequence Synthetic peptide
XQWAVGHXX

SEQ ID NO: 117 - protein, 9 aa, Artificial Sequence
  Description of Artificial Sequence Synthetic peptide
XQWAVGHXG

SEQ ID NO: 118 - protein, 10 aa, Artificial Sequence
  Description of Artificial Sequence Synthetic peptide
RRQWAVGHXL

SEQ ID NO: 119 - protein, 10 aa, Artificial Sequence
  Description of Artificial Sequence Synthetic peptide
RAQWAVGHXL

SEQ ID NO: 120 - protein, 8 aa, Artificial Sequence
  Description of Artificial Sequence Synthetic peptide
QWAVGHXL

SEQ ID NO: 121 - protein, 9 aa, Artificial Sequence
  Description of Artificial Sequence Synthetic peptide
XQWAVGHXX

SEQ ID NO: 122 - protein, 9 aa, Artificial Sequence
  Description of Artificial Sequence Synthetic peptide
XQWAVGHXL

SEQ ID NO: 123 - protein, 9 aa, Artificial Sequence
  Description of Artificial Sequence Synthetic peptide
XQWAVGHXX

SEQ ID NO: 124 - protein, 9 aa, Artificial Sequence
  Description of Artificial Sequence Synthetic peptide
XQWAVGHXL

SEQ ID NO: 125 - protein, 9 aa, Artificial Sequence
  Description of Artificial Sequence Synthetic peptide
XQWAVGHXL

SEQ ID NO: 126 - protein, 9 aa, Artificial Sequence
  Description of Artificial Sequence Synthetic peptide
XQWAVGHXL

SEQ ID NO: 127 - protein, 9 aa, Artificial Sequence
  Description of Artificial Sequence Synthetic peptide
XQWAVAHXX

SEQ ID NO: 128 - protein, 9 aa, Artificial Sequence
  Description of Artificial Sequence Synthetic peptide
XQWAVAHFX

SEQ ID NO: 129 - protein, 9 aa, Artificial Sequence
  Description of Artificial Sequence Synthetic peptide
XQWAVAHFL

SEQ ID NO: 130 - protein, 9 aa, Artificial Sequence
  Description of Artificial Sequence Synthetic peptide
XQWAVAHLL

SEQ ID NO: 131 - protein, 9 aa, Artificial Sequence
  Description of Artificial Sequence Synthetic peptide
XQWAVAHIL

SEQ ID NO: 132 - protein, 9 aa, Artificial Sequence
  Description of Artificial Sequence Synthetic peptide
XQWAVAHLG

SEQ ID NO: 133 - protein, 9 aa, Artificial Sequence
  Description of Artificial Sequence Synthetic peptide
XQWAVAHFX

SEQ ID NO: 134 - protein, 9 aa, Artificial Sequence
  Description of Artificial Sequence Synthetic peptide
XQWAVAHFX

SEQ ID NO: 135 - protein, 9 aa, Artificial Sequence
  Description of Artificial Sequence Synthetic peptide
XQWAVAHFG

SEQ ID NO: 136 - protein, 9 aa, Artificial Sequence
  Description of Artificial Sequence Synthetic peptide
XQWAVAHXG

SEQ ID NO: 137 - protein, 9 aa, Artificial Sequence
  Description of Artificial Sequence Synthetic peptide
XQWAVAHXG

SEQ ID NO: 138 - protein, 9 aa, Artificial Sequence
  Description of Artificial Sequence Synthetic peptide
XQWAVAHFX

SEQ ID NO: 139 - protein, 9 aa, Artificial Sequence
  Description of Artificial Sequence Synthetic peptide
XQWAVAHFL

SEQ ID NO: 140 - protein, 9 aa, Artificial Sequence
  Description of Artificial Sequence Synthetic peptide
XQWAVAHFL

SEQ ID NO: 141 - protein, 9 aa, Artificial Sequence
  Description of Artificial Sequence Synthetic peptide
XQWAVAHIG

SEQ ID NO: 142 - protein, 9 aa, Artificial Sequence
  Description of Artificial Sequence Synthetic peptide
XQWAVGHXX

SEQ ID NO: 143 - protein, 9 aa, Artificial Sequence
  Description of Artificial Sequence Synthetic peptide
XQWAVGHXX

SEQ ID NO: 144 - protein, 9 aa, Artificial Sequence
  Description of Artificial Sequence Synthetic peptide
XQWAVGHXA

SEQ ID NO: 145 - protein, 9 aa, Artificial Sequence
  Description of Artificial Sequence Synthetic peptide
XQWAVGHXA

SEQ ID NO: 146 - protein, 8 aa, Artificial Sequence
  Description of Artificial Sequence Synthetic peptide
QWAVGHXL

SEQ ID NO: 147 - protein, 8 aa, Artificial Sequence
  Description of Artificial Sequence Synthetic peptide
QWAVGHXL

SEQ ID NO: 148 - protein, 9 aa, Artificial Sequence
  Description of Artificial Sequence Synthetic peptide
XQWAVGHXL

SEQ ID NO: 149 - protein, 10 aa, Artificial Sequence
  Description of Artificial Sequence Synthetic peptide
RXQWAVGHXL

SEQ ID NO: 150 - protein, 9 aa, Artificial Sequence
  Description of Artificial Sequence Synthetic peptide
RXQWAVHXL

SEQ ID NO: 151 - protein, 10 aa, Artificial Sequence
  Description of Artificial Sequence Synthetic peptide
RXQWAVGHXL

SEQ ID NO: 152 - protein, 9 aa, Artificial Sequence
  Description of Artificial Sequence Synthetic peptide
XQWAVGHXL

SEQ ID NO: 153 - protein, 8 aa, Artificial Sequence
  Description of Artificial Sequence Synthetic peptide
QWAVGHXL

SEQ ID NO: 154 - protein, 9 aa, Artificial Sequence
  Description of Artificial Sequence Synthetic peptide
XQWAVGHXL

SEQ ID NO: 155 - protein, 9 aa, Artificial Sequence
  Description of Artificial Sequence Synthetic peptide
XQWAVGHXX

SEQ ID NO: 156 - protein, 9 aa, Artificial Sequence
  Description of Artificial Sequence Synthetic peptide
XQWAVGHXL

SEQ ID NO: 157 - protein, 9 aa, Artificial Sequence
  Description of Artificial Sequence Synthetic peptide
XQWAVGHXL

SEQ ID NO: 158 - protein, 10 aa, Artificial Sequence
  Description of Artificial Sequence Synthetic peptide
KAQWAVGHXL

SEQ ID NO: 159 - protein, 10 aa, Artificial Sequence
  Description of Artificial Sequence Synthetic peptide
KAQWAVGHXL

SEQ ID NO: 160 - protein, 10 aa, Artificial Sequence
  Description of Artificial Sequence Synthetic peptide
RSQWAVGHXL

SEQ ID NO: 161 - protein, 10 aa, Artificial Sequence
  Description of Artificial Sequence Synthetic peptide
SSQWAVGHXL

SEQ ID NO: 162 - protein, 10 aa, Artificial Sequence
  Description of Artificial Sequence Synthetic peptide
KSQWAVGHXL

SEQ ID NO: 163 - protein, 10 aa, Artificial Sequence
  Description of Artificial Sequence Synthetic peptide
RSQWAVGHXL

SEQ ID NO: 164 - protein, 10 aa, Artificial Sequence
  Description of Artificial Sequence Synthetic peptide
KAQWAVGHXL

SEQ ID NO: 165 - protein, 9 aa, Artificial Sequence
  Description of Artificial Sequence Synthetic peptide
XQWAVGHXL

SEQ ID NO: 166 - protein, 10 aa, Artificial Sequence
  Description of Artificial Sequence Synthetic peptide
RXQWAVGHXL

SEQ ID NO: 167 - protein, 10 aa, Artificial Sequence
  Description of Artificial Sequence Synthetic peptide
RXQWAVGHXL

SEQ ID NO: 168 - protein, 9 aa, Artificial Sequence
  Description of Artificial Sequence Synthetic peptide
XQWAVGHXL

SEQ ID NO: 169 - protein, 8 aa, Artificial Sequence
  Description of Artificial Sequence Synthetic peptide
QWAVGHXL

SEQ ID NO: 170 - protein, 10 aa, Artificial Sequence
  Description of Artificial Sequence Synthetic peptide
RAQWAVGHXL

SEQ ID NO: 171 - protein, 9 aa, Artificial Sequence
  Description of Artificial Sequence Synthetic peptide
XQWAVGHXX

SEQ ID NO: 172 - protein, 10 aa, Artificial Sequence
  Description of Artificial Sequence Synthetic peptide
SSQWAVGHXL

SEQ ID NO: 173 - protein, 9 aa, Artificial Sequence
  Description of Artificial Sequence Synthetic peptide
XQWAVGHXL

SEQ ID NO: 174 - protein, 9 aa, Artificial Sequence
  Description of Artificial Sequence Synthetic peptide
XQWAVGHXL

SEQ ID NO: 175 - protein, 9 aa, Artificial Sequence
  Description of Artificial Sequence Synthetic peptide
XQWAVGHXX

SEQ ID NO: 176 - protein, 9 aa, Artificial Sequence
  Description of Artificial Sequence Synthetic peptide
XQWAVGHXG

SEQ ID NO: 177 - protein, 9 aa, Artificial Sequence
  Description of Artificial Sequence Synthetic peptide
XQWAVGHXL

SEQ ID NO: 178 - protein, 9 aa, Artificial Sequence
  Description of Artificial Sequence Synthetic peptide
XQWAVGHXG

SEQ ID NO: 179 - protein, 9 aa, Artificial Sequence
  Description of Artificial Sequence Synthetic peptide
XQWAVGHXL

SEQ ID NO: 180 - protein, 9 aa, Artificial Sequence
  Description of Artificial Sequence Synthetic peptide
XQWAVGHXL

SEQ ID NO: 181 - protein, 9 aa, Artificial Sequence
  Description of Artificial Sequence Synthetic peptide
XQWAVGHXX

SEQ ID NO: 182 - protein, 10 aa, Artificial Sequence
  Description of Artificial Sequence Synthetic peptide
RXQWAVGHXL

SEQ ID NO: 183 - protein, 8 aa, Artificial Sequence
  Description of Artificial Sequence Synthetic peptide
QWAVGHXL

SEQ ID NO: 184 - protein, 10 aa, Artificial Sequence
  Description of Artificial Sequence Synthetic peptide
RXQWAVGHXL

SEQ ID NO: 185 - protein, 10 aa, Artificial Sequence
  Description of Artificial Sequence Synthetic peptide
RXQWAVGHXL

SEQ ID NO: 186 - protein, 11 aa, Artificial Sequence
  Description of Artificial Sequence Synthetic peptide
RARQWAVGHXL

SEQ ID NO: 187 - protein, 9 aa, Artificial Sequence
  Description of Artificial Sequence Synthetic peptide
XQWAVGHXX

SEQ ID NO: 188 - protein, 9 aa, Artificial Sequence
  Description of Artificial Sequence Synthetic peptide
XQWAVGHXG

SEQ ID NO: 189 - protein, 10 aa, Artificial Sequence
  Description of Artificial Sequence Synthetic peptide
RRQWAVGHXL

SEQ ID NO: 190 - protein, 10 aa, Artificial Sequence
  Description of Artificial Sequence Synthetic peptide
RAQWAVGHXL

SEQ ID NO: 191 - protein, 8 aa, Artificial Sequence
  Description of Artificial Sequence Synthetic peptide
QWAVGHXL

SEQ ID NO: 192 - protein, 9 aa, Artificial Sequence
  Description of Artificial Sequence Synthetic peptide
XQWAVGHXX

SEQ ID NO: 193 - protein, 9 aa, Artificial Sequence
  Description of Artificial Sequence Synthetic peptide
XQWAVGHXL

SEQ ID NO: 194 - protein, 9 aa, Artificial Sequence
  Description of Artificial Sequence Synthetic peptide
XQWAVGHXX

SEQ ID NO: 195 - protein, 9 aa, Artificial Sequence
  Description of Artificial Sequence Synthetic peptide
XQWAVGHXL

SEQ ID NO: 196 - protein, 9 aa, Artificial Sequence
  Description of Artificial Sequence Synthetic peptide
XQWAVGHXL

SEQ ID NO: 197 - protein, 9 aa, Artificial Sequence
  Description of Artificial Sequence Synthetic peptide
XQWAVGHXL

SEQ ID NO: 198 - protein, 9 aa, Artificial Sequence
  Description of Artificial Sequence Synthetic peptide
XQWAVAHXX

SEQ ID NO: 199 - protein, 9 aa, Artificial Sequence
  Description of Artificial Sequence Synthetic peptide
XQWAVAHFX

SEQ ID NO: 200 - protein, 9 aa, Artificial Sequence
  Description of Artificial Sequence Synthetic peptide
XQWAVAHFL

SEQ ID NO: 201 - protein, 9 aa, Artificial Sequence
  Description of Artificial Sequence Synthetic peptide
XQWAVAHLL

SEQ ID NO: 202 - protein, 9 aa, Artificial Sequence
  Description of Artificial Sequence Synthetic peptide
XQWAVAHIL

SEQ ID NO: 203 - protein, 9 aa, Artificial Sequence
  Description of Artificial Sequence Synthetic peptide
XQWAVAHLG

SEQ ID NO: 204 - protein, 9 aa, Artificial Sequence
  Description of Artificial Sequence Synthetic peptide
XQWAVAHFX

SEQ ID NO: 205 - protein, 9 aa, Artificial Sequence
  Description of Artificial Sequence Synthetic peptide
XQWAVAHFX

SEQ ID NO: 206 - protein, 9 aa, Artificial Sequence
  Description of Artificial Sequence Synthetic peptide
XQWAVAHFG

SEQ ID NO: 207 - protein, 9 aa, Artificial Sequence
  Description of Artificial Sequence Synthetic peptide
XQWAVAHXG

SEQ ID NO: 208 - protein, 9 aa, Artificial Sequence
  Description of Artificial Sequence Synthetic peptide
XQWAVAHXG

SEQ ID NO: 209 - protein, 9 aa, Artificial Sequence
  Description of Artificial Sequence Synthetic peptide
XQWAVAHFX

SEQ ID NO: 210 - protein, 9 aa, Artificial Sequence
  Description of Artificial Sequence Synthetic peptide
XQWAVAHFL

SEQ ID NO: 211 - protein, 9 aa, Artificial Sequence
  Description of Artificial Sequence Synthetic peptide
XQWAVAHFL

SEQ ID NO: 212 - protein, 9 aa, Artificial Sequence
  Description of Artificial Sequence Synthetic peptide
XQWAVAHIG

SEQ ID NO: 213 - protein, 9 aa, Artificial Sequence
  Description of Artificial Sequence Synthetic peptide
XQWAVGHXX

SEQ ID NO: 214 - protein, 9 aa, Artificial Sequence
  Description of Artificial Sequence Synthetic peptide
XQWAVGHXX

SEQ ID NO: 215 - protein, 9 aa, Artificial Sequence
  Description of Artificial Sequence Synthetic peptide
XQWAVGHXA

SEQ ID NO: 216 - protein, 9 aa, Artificial Sequence
  Description of Artificial Sequence Synthetic peptide
XQWAVGHXA

SEQ ID NO: 217 - protein, 8 aa, Artificial Sequence
  Description of Artificial Sequence Synthetic peptide
QWAVGHXL

SEQ ID NO: 218 - protein, 8 aa, Artificial Sequence
  Description of Artificial Sequence Synthetic peptide
QWAVGHXL

SEQ ID NO: 219 - protein, 9 aa, Artificial Sequence
  Description of Artificial Sequence Synthetic peptide
XQWAVGHXL

SEQ ID NO: 220 - protein, 4 aa, Artificial Sequence
  Description of Artificial Sequence Synthetic peptide
VAFG

SEQ ID NO: 221 - protein, 4 aa, Artificial Sequence
  Description of Artificial Sequence Synthetic peptide
VAFG

SEQ ID NO: 222 - protein, 4 aa, Artificial Sequence
  Description of Artificial Sequence Synthetic peptide
VAHG

SEQ ID NO: 223 - protein, 4 aa, Artificial Sequence
  Description of Artificial Sequence Synthetic peptide
VAHG

SEQ ID NO: 224 - protein, 9 aa, Artificial Sequence
  Description of Artificial Sequence Synthetic peptide
XQWAVGHXL

SEQ ID NO: 225 - protein, 10 aa, Artificial Sequence
  Description of Artificial Sequence Synthetic peptide
RXQWAVGHXL

SEQ ID NO: 226 - protein, 10 aa, Artificial Sequence
  Description of Artificial Sequence Synthetic peptide
RXQWAVGHXL

SEQ ID NO: 227 - protein, 10 aa, Artificial Sequence
  Description of Artificial Sequence Synthetic peptide
RXQWAVGHXL

SEQ ID NO: 228 - protein, 10 aa, Artificial Sequence
  Description of Artificial Sequence Synthetic peptide
RXQWAVGHXL

SEQ ID NO: 229 - protein, 4 aa, Artificial Sequence
  Description of Artificial Sequence Synthetic peptide
VAFG

SEQ ID NO: 230 - protein, 4 aa, Artificial Sequence
  Description of Artificial Sequence Synthetic peptide
VAHG

SEQ ID NO: 231 - protein, 7 aa, Unknown
  Description of Unknown Mammalian peptide sequence
QWAVGHL

SEQ ID NO: 232 - protein, 6 aa, Unknown
  Description of Unknown Mammalian peptide sequence
QWAVGH

SEQ ID NO: 233 - protein, 8 aa, Unknown
  Description of Unknown Mammalian peptide sequence
QWAVGAHA

SEQ ID NO: 234 - protein, 7 aa, Unknown
  Description of Unknown Mammalian peptide sequence
QWAVAHF

SEQ ID NO: 235 - protein, 8 aa, Unknown
  Description of Unknown Mammalian peptide sequence
QWAVAHFL

SEQ ID NO: 236 - protein, 8 aa, Unknown
  Description of Unknown Mammalian peptide sequence
QWAVAHLL

SEQ ID NO: 237 - protein, 8 aa, Unknown
  Description of Unknown Mammalian peptide sequence
QWAVAHIL

SEQ ID NO: 238 - protein, 8 aa, Unknown
  Description of Unknown Mammalian peptide sequence
QWAVAHLG

SEQ ID NO: 239 - protein, 8 aa, Unknown
  Description of Unknown Mammalian peptide sequence
QWAVAHFG

SEQ ID NO: 240 - protein, 8 aa, Unknown
  Description of Unknown Mammalian peptide sequence
QWAVAHIG

SEQ ID NO: 241 - protein, 6 aa, Unknown
  Description of Unknown Mammalian peptide sequence
QWAVHL

SEQ ID NO: 242 - protein, 7 aa, Unknown
  Description of Unknown Mammalian peptide sequence
QWAVGHG

SEQ ID NO: 243 - protein, 7 aa, Unknown
  Description of Unknown Mammalian peptide sequence
QWAVGHA

SEQ ID NO: 244 - protein, 6 aa, Unknown
  Description of Unknown Mammalian peptide sequence
QWAVAH

SEQ ID NO: 245 - protein, 7 aa, Unknown
  Description of Unknown Mammalian peptide sequence
QWAVAHG

(19) United States
(12) Patent Application Publication
(10) Pub. No.: US 2009/0317326 A1
(43) Pub. Date: Dec. 24, 2009

(54) RADIOFLUORINATION METHODS

(76) Inventors: Ananth Srinivasan, Berlin (DE); Timo Stellfeld, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/851,940

(22) Filed: Sep. 7, 2007

Related U.S. Application Data
(60) Provisional application No. 60/905,032, filed on Mar. 6, 2007.

(30) Foreign Application Priority Data
Mar. 1, 2007 (EP) 07090035.2
Apr. 23, 2007 (EP) 07090079.0

(51) Int. Cl. A61K 51/08; C07K 7/00; A61K 51/04; A61K 101/02

(52) U.S. Cl. 424/1.69; 530/329; 530/328

(57) ABSTRACT

The present invention relates to radiolabelled substitute benzene compounds for diagnostic imaging. The present invention provides methods for preparation of such compounds, in particular, preparation of novel compounds which serve as precursors for ¹⁸F-labeling, and the use of thus ¹⁸F-labeled compounds for diagnostic imaging.

This application claims the benefit of the filing date of U.S. Provisional Application Ser. No. 60/905,032 filed Mar. 1, 2007, which is incorporated by reference herein.

### FIELD OF INVENTION

This invention relates to novel substitute benzene compounds, which provide access to halogen-labelled, more specifically ¹⁸F-labelled biologically active compounds and the respective halogen-labelled, more specifically ¹⁸F-labelled compounds, methods of preparing such halogen-labelled, more specifically ¹⁸F-labelled compounds, a composition comprising such compounds and their use for diagnostic imaging, a kit comprising a sealed vial containing a predetermined quantity of such novel substitute benzene compounds and such compounds for use as medicament, as diagnostic imaging agent and most specifically as imaging agent for Positron Emission Tomography (PET).

### BACKGROUND

Over the last few years, in-vivo scanning using Positron Emission Tomography (PET) has increased. PET is both a medical and research tool. It is used heavily in clinical oncology for medical imaging of tumors and the search for metastasis, and for clinical diagnosis of certain diffuse brain diseases such as those causing various types of dementias. Radiotracers consisting of a radionuclide stably bound to a biomolecule is used for in vivo imaging of disorders.

In designing an effective radiopharmaceutical tracer for use as a diagnostic agent, it is imperative that the drug has appropriate in vivo targeting and pharmacokinetic properties. Fritzberg et al. (J. Nucl. Med., 1992, 33:394) state further that radionuclide chemistry and associated linkages underscore the need to optimize the attachment and labelling of chemical modifications of the biomolecule carrier, diluent, excipient or adjuvant. Hence the type of radionuclide, the type of biomolecule and the method used for linking them to one another may have a crucial effect onto the radiotracer properties.

Peptides are biomolecules that play a crucial role in many physiological processes including actions as neurotransmitters, hormones, and antibiotics. Research has shown their importance in such fields as neuroscience, immunology, pharmacology, and cell biology. Some peptides can act as chemical messenger. They bind to receptor on the target cell surface and the biological effect of the ligand is transmitted to the target tissue. Hence the specific receptor binding property of the ligand can be exploited by labelling the ligand with a radionuclide. Theoretically, the high affinity of the ligand for the receptor facilitates retention of the radio labelled ligand in receptor expressing tissues. However, it is still under investigation which peptides can efficiently be labelled and under which conditions the labelling shall occur. It is well known that receptor specificity of ligand peptide may be altered during chemical reaction. Therefore an optimal peptidic construct has to be determined.

Tumors overexpress various receptor types to which peptide bound specifically. Boerman et al. (Seminar in Nuclear Medicine, 30(3) July, 2000; pp 195-208) provide a non exhaustive list of peptides binding to receptor involved in tumor, i.e., somatostatin, Vasoactive intestinal peptide (VIP), Bombesin binding to Gastrin-releasing peptide (GRP) receptor, Gastrin, Cholecystokinin (CCK), and Calcitonin.

The radionuclides used in PET scanning are typically isotopes with short half lives such as ¹¹C (˜20 min), ¹³N (˜10 min), ¹⁵O (˜2 min), ⁶⁸Ga (˜68 min) or ¹⁸F (˜110 min). Due to their short half lives, the radionuclides must be produced in a cyclotron which is not too far away in delivery-time from the PET scanner. These radionuclides are incorporated into biologically active compounds or biomolecules that have the function to vehicle the radionuclide into the body though the targeted site, for example a tumor.

The linkage of the radionuclide to the biomolecule is done by various methods resulting in the presence or not of a linker between the radionuclide and the biomolecule. Hence, various linkers are known. C. J. Smith et al. (“Radiochemical investigations of ¹⁷⁷Lu-DOTA-8-Aoc-BBN[7-14]NH₂: an in vitro/in vivo assessment of the targeting ability of this new radiopharmaceutical for PC-3 human prostate cancer cells.” Nucl Med Bio 30(2):101-9; 2003) disclose radiolabeled bombesin wherein the linker is DOTA-X where X is a carbon tether. However, the radiolabel ¹⁷⁷Lu (half life 6.5 days) does not match the biological half-life of the native bombesin what makes the ¹⁷⁷Lu-DOTA-X-bombesin a non-appropriate radiotracer for imaging tumor.

E. Garcia Garayoa et al. (“Chemical and biological characterization of new Re(CO)₃/[^{99m}Tc](CO)₃ bombesin Analogues.” Nucl Med. Biol.; 17-28; 2007) disclose a spacer between the radionuclide [^{99m}Tc] and the bombesin wherein the spacer is −β-Ala-β-Ala- and 3,6-dioxa-8-aminooctanoic acid. E. Garcia Garayoa et al., conclude that the different spacer does not have a significant effect on stability or on receptor affinity.

Listed above linkers have been specifically designed for a specific type of radionuclide and determine the type and chemical conditions of the radiobinding method.

More recently, peptides have been conjugated to a macrocyclic chelator for labelling with ⁶⁴Cu, ⁸⁶Y, and ⁶⁸Ga for PET application. However, such radionuclides interact with the in-vivo catabolism resulting in unwanted physiologic effects and chelate attachment.

¹⁸F-labeled compounds are gaining importance due to the availability thereof as well as due to the development of methods for labeling biomolecules. It has been shown that some compounds labeled with ¹⁸F produce images of high quality. Additionally, the longer lifetime of ¹⁸F would permit longer imaging times and allow preparation of radiotracer batches for multiple patients and delivery of the tracer to other facilities, making the technique more widely available to clinical investigators. Additionally, it has been observed that the development of PET cameras and availability of the instrumentation in many PET centers is increasing. Hence, it is increasingly important to develop new tracers labeled with ¹⁸F.

The nucleophilic aromatic ¹⁸F-fluorination reaction is of great importance for ¹⁸F-labelled radiopharmaceuticals which are used as in vivo imaging agents targeting and visualizing diseases, e.g., solid tumors.

Various methods of radiofluorination have been published using different precursors or starting material for obtaining ¹⁸F-labelled peptides. Due to the smaller size of peptides, both higher target-to-background ratios and rapid blood clearance can often be achieved with radiolabeled peptides. Hence, short-lived positron emission tomography (PET) isotopes are potential candidates for labelling peptides. Among a number of positron-emitting nuclides, fluorine-18 appears to be the best candidate for labelling bioactive peptides by virtue of its favourable physical and nuclear characteristics. The major disadvantage of labelling peptides with ¹⁸F is the laborious and time-consuming preparation of the ¹⁸F labelling agents. Due to the complex nature of peptides and several functional groups associated with the primary structure, ¹⁸F-labelled peptides are not prepared by direct fluorination. Hence, difficulties associated with the preparation of ¹⁸F-labeled peptide were alleviated with the employment of prosthetic groups as shown below. Several such prosthetic groups have been proposed in the literature, including N-succinimidyl-4-[¹⁸F]fluorobenzoate, m-maleimido-N-(p-[¹⁸F]fluorobenzyl)-benzamide, N-(p-[¹⁸F]fluorophenyl) maleimide, and 4-[¹⁸F]fluorophenacylbromide. Almost all of the methodologies currently used today for the labeling of peptides and proteins with ¹⁸F utilize active esters of the fluorine labeled synthon.

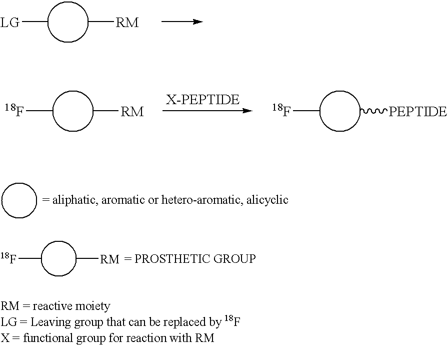

Okarvi et al. (“Recent progress in fluorine-18 labelled peptide radiopharmaceuticals.” Eur J. Nucl. Med., 2001 Jul. 28(7):929-38)) present a review of the recent developments in ¹⁸F-labelled biologically active peptides used in PET.

Xianzhong Zhang et al. (“¹⁸F-labeled bombesin analogs for targeting GRP receptor-expressing prostate cancer,” J. Nucl. Med., 47(3):492-501 (2006)) relate to the 2-step method detailed above. [Lys3]Bombesin ([Lys3]BBN) and aminocaproic acid-bombesin(7-14) (Aca-BBN(7-14)) were labeled with ¹⁸F by coupling the Lys3 amino group and Aca amino group, respectively, with N-succinimidyl-4-¹⁸F-fluorobenzoate (¹⁸F-SFB) under slightly basic condition (pH 8.5). Unfortunately, the obtained ¹⁸F-FB-[Lys3]BBN is metabolically relatively unstable having for result to reduce the extent of use of the ¹⁸F-FB-[Lys3]BBN for reliable imaging of tumor.

Thorsten Poethko et al. (“Two-step methodology for high-yield routine radiohalogenation of peptides: ¹⁸F-labeled RGD and octreotide analogs.” J. Nucl. Med., 2004 May; 45(5):892-902) relate to a 2-step method for labelling RGD and octreotide analogs. The method discloses the steps of radiosynthesis of the ¹⁸F-labeled aldehyde or ketone and the chemoselective ligation of the ¹⁸F-labeled aldehyde or ketone to the aminooxy functionalized peptide.

Thorsten Poethko et al. (“First ¹⁸F-labeled tracer suitable for routine clinical imaging of socmatostatin receptor-expressing tumors using positron emission tomography.” Clin. Cancer Res., 2004 June 1; 10(11):3593-606) apply the 2-step method for the synthesis of ¹⁸F-labeled carbohydrated Tyr(3)-octreotate (TOCA) analogs with optimized pharmacokinetics suitable for clinical routine somatostatin-receptor (sst) imaging.

WO 2003/080544 A1 and WO 2004/080492 A1 relate to radiofluorination methods of bioactive peptides for diagnostics imaging using the 2-step method shown above.

The most crucial aspect in the successful treatment of any cancer is early detection. Likewise, it is crucial to properly diagnose the tumor and metastasis.

Routine application of ¹⁸F-labeled peptides for quantitative in vivo receptor imaging of receptor-expressing tissues and quantification of receptor status using PET is limited by the lack of appropriate radiofluorination methods for routine large-scale synthesis of ¹⁸F-labeled peptides. There is a clear need for radiofluorination method that can be conducted rapidly without loss of receptor affinity by the peptide and leading to a positive imaging (with reduced background), wherein the radiotracer is stable and shows an enhanced clearance properties

The conversions of mono-(mainly para-) substituted phenyl-trimethylammonium derivatives to substituted [¹⁸F]-fluorobenzene derivatives which serve as radiopharmaceutical itself or as prosthetic group for the ¹⁸F-labeling of small and large molecules have been reported in the literature (Irie et al., 1982, Fluorine Chem., 27, (1985), 117-191; Haka et al, 1989) (see scheme 1).

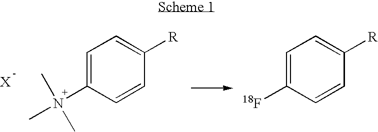

There are only a few publications about nucleophilic aromatic ¹⁸F-fluorination reactions of trimethylammonium-substituted aromatic derivatives which contain two or more substituents beside the trimethylammonium moiety:

Oya et al., treated [2-chloro-5-(2-dimethylcarbamoyl-phenylsulfanyl)-4-nitro-phenyl]-trimethylammonium triflate with [¹⁸F]potassium fluoride and obtained the desired ¹⁸F-labelled compound (Journal of Medicinal Chemistry (2002), 45(21), 4716-4723).

Li et al. reported on the ¹⁸F-fluorination reaction of 4-(N,N,N-trimethylammonium)-3-cyano-3′-iodobenzophenone triflate (Bioconjugate Chemistry (2003), 14(2), 287-294).

Enas et al. converted (2,2-dimethyl-1,3-dioxo-indan-5-yl)-trimethylammonium triflate into the desired ¹⁸F-labelled compound (Journal of Fluorine Chemistry, (1993), 63(3), 233-41).

Seimbille et al. and other groups labelled (2-chloro-4-nitro-phenyl)-trimethylammonium triflate successfully with ¹⁸F (J. Labelled Compd. Radiopharm., (2005), 48, 11, 829-843).

(2-Benzyloxy-4-formyl-phenyl)-trimethylammonium triflate was successfully labelled with ¹⁸F at high temperature (130° C.) by Langer et, al. (Bioorg. Med. Chem., EN, 9, 3, 2001, 677-694).

Lang et al. radiolabelled trimethyl-(2-methyl-4-pentamethylphenyl methoxycarbonyl-phenyl)-ammonium triflate by use of [¹⁸F]potassium fluoride (J. Med. Chem., 42, 9, 1999, 1576-1586).

Trimethyl-(4-nitro-naphthalen-1-yl)-ammonium triflate was labelled with ¹⁸F by Amokhtari et al. (J. Labelled Compd. Radiopharm., S42, 1, (1999), S622-S623).

Lemaire et al. converted (2-formyl-5-methoxy-phenyl)-trimethylammonium triflate into the desired ¹⁸F-labelled product (J. Labelled Compd. Radiopharm., 44, 2001, S857-S859).

VanBrocklin et al. describe the ¹⁸F labeling of (2-bromo-4-nitro-phenyl)-trimethyl-ammonium triflate (J. Labelled Compd. Radiopharm., 44, 2001, S880-S882).

Cetir Centre Medic report on the successful ¹⁸F-labeling of (5-chloro-8-hydroxy-quinolin-7-yl)-trimethylammonium triflate (EP 1 563 852 A1).

Most of these mentioned ¹⁸F-labelled aromatic derivatives which contain two or more additional substituents cannot be coupled to chemical functionalities like amines, thiols, carboxylic acids, phenols or other chemicals groups of complex molecules like peptides without further transformations.

¹⁸F-labelings of more complex radiopharmaceuticals like peptides take place in all known publications in a two- or multi-step strategy (see scheme 2, overview: Eur. J. Nucl. Med., (2001), 28, 929-938).

For these kinds of ¹⁸F-labeling also mono-substituted phenyl-trimethylammonium derivatives are used and react in a first step with [¹⁸F]potassium fluoride to obtain substituted [¹⁸F]-fluorobenzene derivatives. These compounds are then coupled in a second step to larger and more complex molecules like peptides or nucleotides (see scheme 2).

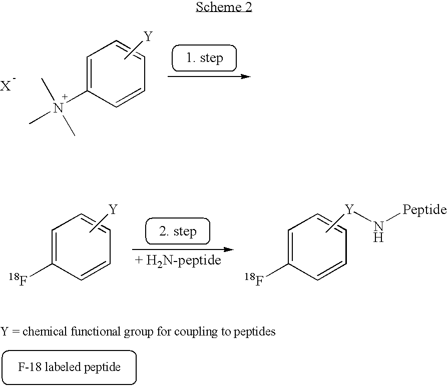

Especially 4-[¹⁸F]fluorobenzaldehyde has been used in many examples for F-18 labeling of complex molecules (e.g., Journal of Nuclear Medicine, (2004), 45(5), 892-902). But also N-succinimidyl-8-[4′-[¹⁸F]fluorobenzylamino]suberate (Bioconjugate Chem., (1991), 2, 44-49), 4-[¹⁸F]fluorophenacyl bromide and 3-[¹⁸F]fluoro-5-nitrobenzimidate (J. Nucl Med., (1987), 28, 462-470), m-maleimido-N-(p-[¹⁸F]fluorobenzyl)-benzamide (J. Labelled Compd. Radiopharm., (1989), 26, 287-289,), N-{4-[4-[¹⁸F]fluorobenzylidene(aminooxy)-butyl}-maleimide (Bioconjugate Chem., (2003), 14, 1253-1259), [¹⁸F]N-(4-fluorobenzyl)-2-bromoacetamide (Bioconjugate Chem., (2000), 11, 627-636) and [¹⁸F]-3,5-difluorophenyl azide (and 5 derivatives) (J. Org. Chem., (1995), 60, 6680-6681) are known examples. F-18 labeling of peptides via para-[¹⁸F]-fluorobenzoates is also a very common method either by coupling of the corresponding acid with additional activating agents (such as 1,3-dicyclohexylcarbodiimide/1-hydroxy-7-azabenzotriazole (DCC/HOAt) or N-[(dimethylamino)-1H-1,2,3-triazolyl[4,5]pyridine-1-yl-methylene]-N-methyl-methan-aminium hexafluorophosphate N-oxide (HATU/DIPEA, Eur. J. Nucl. Med. Mol. Imaging., (2002), 29, 754-759) or by isolated N-succinimidyl 4-[¹⁸F]fluorobenzoate (Nucl. Med. Biol., (1996), 23, 365).

As outlined above, the current state of art provides the trimethylammonium group and the nitro group as the sole leaving groups to afford ¹⁸F-labelled compounds for both indirect labeling of peptides via prosthetic groups (references above), direct labeling of peptides as well as for small molecules (see EP 06090166) not published at the date of filing.

Further references:
WO 2004/080492 A1, “Methods of radiofluorination of biologically active vectors” Published 23 Sep. 2004.
K. Bruus-Jensen, T. Poethko, M. Schottelius, A. Hauser, M. Schwaiger, H. J. Wester: “Chemoselective hydrazones formation between HYNIC-functionalized peptides and (18)F-fluorinated aldehydes.” Nucl Med. Biol., (2006) 33(2):173-83.
T. Poethko, M. Schottelius, G. Thurnshirn, U. Hersel, M. Herz, G. Henriksen, H. Kessler, M. Schwaiger, H. J. Wester: “Two-step methodology for high-yield routine radiohalogenation of peptides, (18)F-labelled RGD and octreotide analogs.” J Nucl Med., 2004 May, 45(5):892-902 and references therein.
Zhang X, Cai W, Cao F, Schreibmann E, Wu Y, Wu J. C, Xing L, Chen X. “¹⁸F-labelled bombesin analogs for targeting GRP receptor-expressing prostate cancer.” J Nucl. Med. (2006), 47(3):492-501.
Z. Li, Y. S. Ding, A. Gifford, J. S. Fowler, J. S. Gatley. “Synthesis of structurally identical fluorine-18 and iodine isotope labeling compounds for comparative imaging” Bioconjug Chem., (2003), 14(2):287-94.

For a number of these diagnostic imaging compounds it would be detrimental for their targeting activity to be subject to harsh reaction conditions during radiolabeling like, e.g., high temperatures which are usually used during nucleophilic aromatic ¹⁸F-fluorination reaction. That is why in the prior art, e.g., peptides are labelled via a two step approach as outlined above. This two step approach is time consuming and requires multiple purification steps. Displacement of the trimethylammonium and/or nitro leaving groups is accomplished at elevated temperatures and hence it is desirable to provide alternative leaving groups to accomplish the ¹⁸F incorporation under milder conditions compatible with chemical and biological stability of the targeting agent. Due to the limited half life of the ¹⁸F isotope of about only 111 minutes, there is a high need for compounds and methods that allow provision of the ¹⁸F-radiolabelled compound with less steps needed.

The problem to be solved by the present invention is the provision of compounds and methods that allow for radiolabeling compounds with halogen, more specifically with ¹⁸F, in a one-step approach.

### SUMMARY OF THE INVENTION

A first aspect of the present invention refers to novel substitute benzene compounds having general chemical Formula A, wherein K=LG-O (general chemical Formula I), and to pharmaceutically acceptable salts, hydrates, esters, amides, solvates and prodrugs thereof. These compounds are precursors to the novel substitute benzene compounds according to the second aspect of the present invention.

A second aspect of the present invention refers to novel substitute benzene compounds having general chemical Formula A, wherein K=W (general chemical Formula II), and to pharmaceutically acceptable salts, hydrates, esters, amides, solvates and prodrugs thereof.

Compounds having general chemical Formula A, wherein K=LG-O (general chemical Formula I), can be converted into compounds having general chemical Formula A, wherein K=W (general chemical Formula II), by means of a one-step labeling more preferably radiolabeling reaction with a fluorine isotope more specifically with ¹⁸F.

A third aspect of the present invention refers to a one-step method of labeling more preferably radiolabeling radiofluorinating compounds having general chemical Formula A, wherein K=LG-O, in order to arrive at compounds having general chemical Formula A, wherein K=W.

A fourth aspect of the present invention refers to compositions, more preferably to diagnostic compositions, comprising a compound having general chemical Formula A, wherein K=LG-O, or a pharmaceutically acceptable salt, hydrate, ester, amide, solvate or prodrug thereof and a pharmaceutically acceptable carrier, diluent, excipient or adjuvant. According to this fourth aspect the present invention further refers to compositions, more preferably diagnostic compositions, comprising a radiolabelled compound having general chemical Formula A, wherein K=W, or a pharmaceutically acceptable salt, hydrate, ester, amide, solvate or prodrug thereof and a pharmaceutically acceptable carrier, diluent, excipient or adjuvant.

A fifth aspect of the present invention refers to a method of imaging diseases, the method comprising introducing into a patient a detectable quantity of a labelled compound having general chemical Formula A, wherein K=W, or a pharmaceutically acceptable salt, hydrate, ester, amide, solvate or prodrug thereof.

A sixth aspect of the present invention refers to a kit for preparing a radiopharmaceutical preparation, said kit comprising a sealed vial containing a predetermined quantity of the compound of Formula A, wherein K=LG-O, or a pharmaceutically acceptable salt, hydrate, ester, amide, solvate or prodrug thereof.

A seventh aspect of the present invention refers to a compound having general chemical Formula A, wherein K=LG-O or W, or of a pharmaceutically acceptable salt, hydrate, ester, amide, solvate or prodrug thereof for use as medicament and, if K=W, for use as diagnostic imaging agent and more specifically for use as imaging agent for PET.

An eighth aspect of the present invention refers to a use of a compound having general chemical Formula A, wherein K=LG-O or W, or of a pharmaceutically acceptable salt, hydrate, ester, amide, solvate or prodrug thereof for the manufacture of a medicament, more specifically for the manufacture of a diagnostic imaging agent and most specifically for the manufacture of a diagnostic imaging agent for imaging tissue at a target site using the imaging agent.

Further aspects of the present invention refer to methods and intermediates useful for synthesizing the tumor imaging compounds of Formula A, wherein K=LG-O or W, as described herein.

### DETAILED DESCRIPTION OF THE INVENTION

As used hereinafter in the description of the invention and in the claims, the term “alkyl”, by itself or as part of another group, refers to a straight chain or branched chain alkyl group with 1 to 20 carbon atoms such as, for example methyl, ethyl, propyl, isopropyl, butyl, isobutyl, tert-butyl, pentyl, isopentyl, neopentyl, heptyl, hexyl, decyl. Alkyl groups can also be substituted, such as by halogen atoms, hydroxyl groups, C₁-C₄ alkoxy groups or C₆-C₁₂ aryl groups (which, intern, can also be substituted, such as by 1 to 3 halogen atoms). More preferably alkyl is C₁-C₁₀ alkyl, C₁-C₆ alkyl or C₁-C₄ alkyl.

As used hereinafter in the description of the invention and in the claims, the term “cycloalkyl” by itself or as part of another group, refers to mono- or bicyclic chain of alkyl group with 3 to 20 carbon atoms such as, for example cyclopropyl, cyclobutyl, cyclopentyl, cyclohexyl or cycloheptyl. More preferably cycloalkyl is C₃-C₁₀ cycloalkyl or C₅-C₈ cycloalkyl, most preferably C₆ cycloalkyl.

As used hereinafter in the description of the invention and in the claims, the term “heterocycloalkyl”, by itself or as part of another group, refers to groups having 3 to 20 mono- or bi-ring atoms of a cycloalkyl; and containing carbon atoms and 1, 2, 3 or 4 oxygen, nitrogen or sulfur heteroatoms. More preferably heterocycloalkyl is C₃-C₁₀ heterocycloalkyl, C₅-C₈ heterocycloalkyl or C₅-C₁₄ heterocycloalkyl, most preferably C₆ heterocycloalkyl.

As used hereinafter in the description of the invention and in the claims, the term “aralkyl” refers to aryl-substituted alkyl radicals such as benzyl, diphenylmethyl, triphenylmethyl, phenylethyl, phenylbutyl and diphenylethyl.

As used hereinafter in the description of the invention and in the claims, the terms “aryloxy” refers to aryl groups having an oxygen through which the radical is attached to a nucleus, examples of which are phenoxy.

As used hereinafter in the description of the invention and in the claims, the terms “alkenyl” and “alkynyl” are similarly defined as for alkyl, but contain at least one carbon-carbon double or triple bond, respectively. More preferably C₂-C₆ alkenyl and C₂-C₆ alkynyl.

As used hereinafter in the description of the invention and in the claims, the term “lower unbranched or branched alkyl” shall have the following meaning: a substituted or unsubstituted, straight or branched chain monovalent or divalent radical consisting substantially of carbon and hydrogen, containing no unsaturation and having from one to eight carbon atoms, e.g., but not limited to methyl, ethyl, n-propyl, n-pentyl, 1,1-dimethylethyl (t-butyl), n-heptyl and the like.

As used hereinafter in the description of the invention and in the claims, the terms “aralkenyl” refers to aromatic structure (aryl) coupled to alkenyl as defined above.

As used hereinafter in the description of the invention and in the claims, the terms “alkoxy (or alkyloxy), aryloxy, and aralkenyloxy” refer to alkyl, aryl, and aralkenyl groups respectively linked by an oxygen atom, with the alkyl, aryl, and aralkenyl portion being as defined above.

As used hereinafter in the description of the invention and in the claims, the terms “inorganic acid” and “organic acid”, refer to mineral acids, including, but not being limited to: acids such as carbonic, nitric, phosphoric, hydrochloric, perchloric or sulphuric acid or the acidic salts thereof such as potassium hydrogen sulphate, or to appropriate organic acids which include, but are not limited to: acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulfonic, ethanesulfonic, benzenesulfonic, phantothenic, toluenesulfonic, trifluormethansulfonic and sulfanilic acid, respectively.

As used hereinafter in the description of the invention and in the claims, the term “aryl”, by itself or as part of another group, refers to monocyclic or bicyclic aromatic groups containing from 6 to 12 carbon atoms in the ring portion, preferably 6-10 carbons in the ring portion, such as phenyl, naphthyl or tetrahydronaphthyl.

As used hereinafter in the description of the invention and in the claims, the term “heteroaryl”, by itself or as part of another group, refers to groups having 5 to 14 ring atoms, 6, 10 or 14 π electrons shared in a cyclic array; and containing carbon atoms and 1, 2, 3 or 4 oxygen, nitrogen or sulfur heteroatoms. Examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxythiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, carbolinyl, phenanthridinyl, acridinyl, perimidinyl, phenanthrolinyl, phenazinyl, isothiazolyl, phenothiazinyl, isoxazolyl, furazanyl und phenoxazinyl.

Whenever the term substituted is used, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a pharmaceutical composition. The substituent groups may be selected from halogen atoms, hydroxyl groups, C₁-C₄ alkoxy groups or C₆-C₁₂ aryl groups (which, intern, can also be substituted, such as by 1 to 3 halogen atoms).

As used hereinafter in the description of the invention and in the claims, the term “fluorine isotope” (F) refers to all isotopes of the fluorine atomic element. Fluorine isotope (F) is selected from radioactive or non-radioactive isotope. The radioactive fluorine isotope is selected from ¹⁸F. The non-radioactive “cold” fluorine isotope is selected from ¹⁹F.

As used hereinafter in the description of the invention and in the claims, the term “prodrug” means any covalently bonded compound, which releases the active parent pharmaceutical according to formula II.

The term “prodrug” as used throughout this text means the pharmacologically acceptable derivatives such as esters, amides and phosphates, such that the resulting in vivo biotransformation product of the derivative is the active drug as defined in the compounds of formula (I). The reference by Goodman and Gilman (The Pharmaco-logical Basis of Therapeutics, 8 ed, McGraw-HiM, Int. Ed, 1992, “Biotransformation of Drugs”, p 13-15) describing prodrugs generally is hereby incorporated. Prodrugs of a compound of the present invention are prepared by modifying functional groups present in the compound in such a way that the modifications are cleaved, either in routine manipulation or in vivo, to the parent compound. Prodrugs of the compounds of the present invention include those compounds wherein for instance a hydroxy group, such as the hydroxy group on the asymmetric carbon atom, or an amino group is bonded to any group that, when the prodrug is administered to a patient, cleaves to form a free hydroxyl or free amino, respectively.

Typical examples of prodrugs are described for instance in WO 99/33795, WO 99/33815, WO 99/33793 and WO 99/33792 all incorporated herein by reference.

Prodrugs are characterized by excellent aqueous solubility, increased bioavailability and are readily metabolized into the active inhibitors in vivo.

As used hereinafter in the description of the invention and in the claims, the term “amino acid sequence” is defined herein as a polyamide obtainable by (poly)condensation of at least two amino acids.

As used hereinafter in the description of the invention and in the claims, the term “amino acid” means any molecule comprising at least one amino group and at least one carboxyl group, but which has no peptide bond within the molecule. In other words, an amino acid is a molecule that has a carboxylic acid functionality and an amine nitrogen having at least one free hydrogen, preferably in alpha position thereto, but no amide bond in the molecule structure. Thus, a dipeptide having a free amino group at the N-terminus and a free carboxyl group at the C-terminus is not to be considered as a single “amino acid” in the above definition. The amide bond between two adjacent amino acid residues which is obtained from such a condensation is defined as “peptide bond”. Optionally, the nitrogen atoms of the polyamide backbone (indicated as NH above) may be independently alkylated, e.g., with C₁-C₆-alkyl, preferably CH₃.

An amide bond as used herein means any covalent bond having the structure

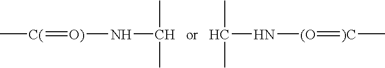

wherein the carbonyl group is provided by one molecule and the NH-group is provided by the other molecule to be joined. The amide bonds between two adjacent amino acid residues which are obtained from such a polycondensation are defined as “peptide bonds”. Optionally, the nitrogen atoms of the polyamide backbone (indicated as NH above) may be independently alkylated, e.g., with —C₁-C₆-alkyl, preferably —CH₃.

As used hereinafter in the description of the invention and in the claims, an amino acid residue is derived from the corresponding amino acid by forming a peptide bond with another amino acid.

As used hereinafter in the description of the invention and in the claims, an amino acid sequence may comprise naturally occurring and/or synthetic amino acid residues, proteinogenic and/or non-proteinogenic amino acid residues. The non-proteinogenic amino acid residues may be further classified as (a) homo analogues of proteinogenic amino acids, (b) β-homo analogues of proteinogenic amino acid residues and (c) further non-proteinogenic amino acid residues.

Accordingly, the amino acid residues may be derived from the corresponding amino acids, e.g., from
proteinogenic amino acids, namely Ala, Arg, Asn, Asp, Cys, Gln, Glu, Gly, His, Ile, Leu, Lys, Met, Phe, Pro, Ser, Thr, Trp, Tyr and Val; or
non-proteinogenic amino acids, such as
   homo analogues of proteinogenic amino acids wherein the sidechain has been extended by a methylene group, e.g., homoalanine (Hal), homoarginine (Har), homocysteine (Hcy), homoglutamine (Hgl), homohistidine (Hhi), homoisoleucine (Hil), homoleucine (Hle), homolysine (Hly), homomethionine (Hme), homophenylalanine (Hph), homoproline (Hpr), homoserine (Hse), homothreonine (Hth), homotryptophane (Htr), homotyrosine (Hty) and homovaline (Hva);
   β-homo analogues of proteinogenic amino acids wherein a methylene group has been inserted between the α-carbon and the carboxyl group yielding β-amino acids, e.g. β-homoalanine (βHal), β-homoarginine (βHar), β-homoasparagine (βHas), β-homocysteine (βHcy), β-homoglutamine (βHgl), β-homohistidine (βHhi), β-homoisoleucine (βHil), β-homoleucine (βHle), β-homolysine (βHly), β-homomethionine (βHme), β-homophenylalanine (βHph), β-homoproline (βHpr), β-homoserine (βHse), β-homothreonine (βHth), β-homotryptophane (βHtr), β-homotyrosine (βHty) and β-homovaline (βHva);
   further non-proteinogenic amino acids, e.g. α-aminoadipic acid (Aad), β-aminoadipic acid (βAad), α-aminobutyric acid (Abu), α-aminoisobutyric acid (Aib), β-alanine (βAla), 4-aminobutyric acid (4-Abu), 5-aminovaleric acid (5-Ava), 6-aminohexanoic acid (6-Ahx), 8-aminooctanoic acid (8-Aoc), 9-aminononanoic acid (9-Anc), 10-aminodecanoic acid (10-Adc), 12-aminododecanoic acid (12-Ado), α-aminosuberic acid (Asu), azetidine-2-carboxylic acid (Aze), β-ayclohexylalanine (Cha), aitruiline (Cit), dehydroalanine (Dha), γ-carboxyglutamic acid (Gla), α-cyclohexylglycine (Chg), propargylglycine (Pra), pyroglutamic acid (Glp), α-tert-butylglycine (Tle), 4-benzoylphenylalanine (Bpa), δ-hydroxylysine (Hyl), 4-hydroxyproline (Hyp), allo-isoleucine (alle), lanthionine (Lan), (1-naphthyl)alanine (1-NaI), (2-naphthyl)alanine (2-NaI), norleucine (NIe), norvaline (Nva), ornithine (Orn), phenylglycin (Phg), pipecolic acid (Pip), sarcosine (Sar), selenocysteine (Sec), statine (Sta), β-thienylalanine (Thi), 1,2,3,4-tetrahydroisochinoline-3-carboxylic acid (Tic), allo-threonine (aThr), thiazolidine-4-carboxylic acid (Thz), γ-aminobutyric acid (GABA), iso-cysteine (iso-Cys), diaminopropionic acid (Dpr), 2,4-diaminobutyric acid (Dab), 3,4-diaminobutyric acid (γβDab), biphenylalanine (Bip), phenylalanine substituted in para-position with —C₁-C₆ alkyl, -halide, —NH₂, —CO₂H or Phe(4-R) (wherein R=—C₁-C₆ alkyl, -halide, —NH₂, or —CO₂H); peptide nucleic acids (PNA, cf. P. E. Nielsen, Acc. Chem. Res., 32, 624-30);
or their N-alkylated analogues, such as their N-methylated analogues.

Cyclic amino acids may be proteinogenic or non-proteinogenic, such as Pro, Aze, Gip, Hyp, Pip, Tic and Thz.

For further examples and details reference can be made to, e.g., J. H. Jones, J. Peptide Sci., 2003, 9, 1-8 which is herein incorporated by reference.

As used hereinafter in the description of the invention and in the claims, the terms “non-proteinogenic amino acid” and “non-proteinogenic amino acid residue” also encompass derivatives of proteinogenic amino acids. For example, the side chain of a proteinogenic amino acid residue may be derivatized thereby rendering the proteinogenic amino acid residue “non-proteinogenic”. The same applies to derivatives of the C-terminus and/or the N-terminus of a proteinogenic amino acid residue terminating the amino acid sequence.

As used hereinafter in the description of the invention and in the claims, a proteinogenic amino acid residue is derived from a proteinogenic amino acid selected from the group consisting of Ala, Arg, Asn, Asp, Cys, Gln, Glu, Gly, His, lie, Leu, Lys, Met, Phe, Pro, Ser, Thr, Trp, Tyr and Val either in L- or D-configuration; the second chiral center in Thr and Ile may have either R- or S-configuration. Therefore, for example, any posttranslational modification of an amino acid sequence, such as N-alkylation, which might naturally occur renders the corresponding modified amino acid residue “non-proteinogenic”, although in nature said amino acid residue is incorporated in a protein, Preferably modified amino acids are selected from N-alkylated amino acids, β-amino acids, γ-amino acids, lanthionines, dehydro amino acids, and amino acids with alkylated guanidine moieties.

As used hereinafter in the description of the invention and in the claims, the term “peptidomimetic” relates to molecules which are related to peptides, but with different properties. A peptidomimetic is a small protein-like chain designed to mimic a peptide.

They typically arise from modification of an existing peptide in order to alter the molecule's properties. For example, they may arise from modifications to change the molecule's stability or biological activity. This can have a role in the development of drug-like compounds from existing peptides. These modifications involve changes to the peptide that will not occur naturally.

As used hereinafter in the description of the invention and in the claims, the term “peptide analogs”, by itself refers to synthetic or natural compounds which resemble naturally occurring peptides in structure and/or function.

As used hereinafter in the description of the invention and in the claims, the term “pharmaceutically acceptable salt” relates to salts of inorganic and organic acids, such as mineral acids, including, but not limited to, acids such as carbonic, nitric or sulfuric acid, or organic acids, including, but not limited to acids such as aliphatic, cycloaliphatic, aromatic, araliphatic, heterocyclic, carboxylic and sulphonic acids, examples of which are formic, acetic, trifluoroacetic, propionic, succinic, glycolic, gluconic, lactic, malic, fumaric, pyruvic, benzoic, anthranilic, mesylic, salicylic, phenylacetic, mandelic, embonic, methansulfonic, ethanesulfonic, benzenesulfonic, phantothenic, toluenesulfonic and sulfanilic acid.

If a chiral center or another form of an isomeric center is present in a compound having general chemical Formulae A, I, II, III or IV of the present invention, as given hereinafter, all forms of such isomers, including enantiomers and diastereoisomers, are intended to be covered herein. Compounds containing a chiral center may be used as a racemic mixture or as an enantiomerically enriched mixture, or the racemic mixture may be separated using well-known techniques and an individual enantiomer maybe used alone. In cases in which compounds have unsaturated carbon-carbon double bonds, both the cis-isomer and trans-isomers are within the scope of this invention. In cases in which compounds may exist in tautomeric forms, such as keto-enol tautomers, each tautomeric form is contemplated as being included within the scope of the present invention whether existing in equilibrium or predominantly in one form.

As used hereinafter in the description of the invention and in the claims, the term “oligonucleotide” shall have the following meaning: short sequences of nucleotides typically with twenty or fewer bases. Examples are, but are not limited to, molecules named and cited in the book: “The aptamers handbook. Functional oligonuclides and their applicaton” by Svenn Klussmann, Wiley-VCH, 2006. An example for such an oligonucleotide is TTA1 (J. Nucl. Med., 2006, April, 47(4):668-78).

As used hereinafter in the description of the invention and in the claims, the term “aptamer” refers to an oligonucleotide, comprising from 4 to 100 nucleotides, wherein at least two single nucleotides are connected to each other via a phosphodiester linkage. Said aptamers have the ability to bind specifically to a target molecule (see e.g., M Famulok, G Mayer, “Aptamers as Tools in Molecular Biology and Immunology”, in: “Combinatorial Chemistry in Biology, Current Topics in Microbiology and Immunology” (M Famulok, C H Nong, E L Winnacker, Eds.), Springer Verlag Heidelberg, 1999, Vol. 243, 123-136), There are many ways known to the skilled person of how to generate such aptamers that have specificity for a certain target molecule. An example is given in WO 01/09390 A, the disclosure of which is hereby incorporated by reference. Said aptamers may comprise substituted or non-substituted natural and non-natural nucleotides. Aptamers can be synthesized in vitro using, e.g., an automated synthesizer. Aptamers according to the present invention can be stabilized against nuclease degradation, e.g., by the substitution of the 2′-OH group versus a 2′-fluoro substituent of the ribose backbone of pyrimidine and versus 2′-O-methyl substituents in the purine nucleic acids. In addition, the 3′ end of an aptamer can be protected against exonuclease degradation by inverting the 3′ nucleotide to form a new 5′-OH group, with a 3′ to 3′ linkage to a penultimate base.

For the purpose of this invention, the term “nucleotide” refers to molecules comprising a nitrogen-containing base, a 5-carbon sugar, and one or more phosphate groups. Examples of said base comprise, but are not limited to, adenine, guanine, cytosine, uracil, and thymine. Also non-natural, substituted or non-substituted bases are included. Examples of 5-carbon sugar comprise, but are not limited to, D-ribose, and D-2-desoxyribose. Also other natural and non-natural, substituted or non-substituted 5-carbon sugars are included. Nucleotides as used in this invention may comprise from one to three phosphates.

As used hereinafter in the description of the invention and in the claims, the term “halogen” refers to F, Cl, Br and I.

In a first aspect the present invention refers to compounds having general chemical Formula A, wherein K=LG-O (general chemical Formula I):

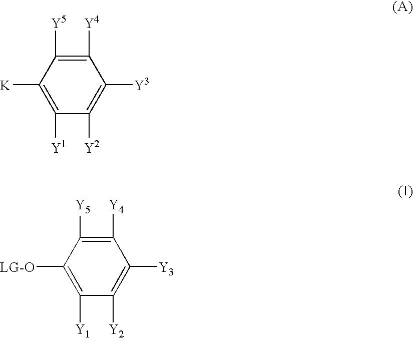

wherein:
LG is a leaving group suitable for displacement by means of a nucleophilic aromatic substitution reaction, K is LG-O wherein —O is involved in the nucleophilic aromatic substitution and form with LG a known leaving entity for the skilled person;
one of —Y¹, —Y², —Y³, —Y⁴ and —Y⁵ is a First Substituent (-G) which is selected from the group comprising —H, —F, —Cl, —Br, —I, —NO, —NO₂, —NR⁴COCF₃, —NR⁴SO₂CF₃, —N(CF₃)₂, —NHCSNHR⁴, —N(SO₂R⁵)₂, —N(O)═NCONH₂, —NR⁴CN, —NHCSR⁵, —N≡C, —N═C(CF₃)₂, —N═NCF₃, —N═NCN, —NR⁴COR⁴, —NR⁴COOR⁵, —OSO₂CF₃, —OSO₂C₆H₅, —OCOR⁵, —ONO₂, —OSO₂R⁵, —O—C═CH₂, —OCF₂CF₃, —OCOCF₃, —OCN, —OCF₃, —C≡N, —C(NO₂)₃, —COOR⁴, —CONR⁴R⁵, —C(S)NH₂, —CH═NOR⁴, —CH₂SO₂R⁴, —COCF₃, —CF₃, —CF₂Cl—CBr₃, —CClF₂, —CCl₃, —CF₂CF₃, —C≡CR⁴, —CH═NSO₂CF₃, —CH₂CF₃, —COR⁵, —CH═NOR⁵, —CH₂CONH₂—CSNHR⁵, —CH═NNHCSNH₂, —CH═NNHCONHNH₂, —C≡CF₃, —CF═CFCF₃, —CF₂—CF₂—CF₃, —CR⁴(CN)₂, —COCF₂CF₂CF₃, —C(CF₃)₃, —C(CN)₃, —CR⁴═C(CN)₂, −1-pyrryl, —C(CN)═C(CN)₂, —C-pyridyl, —COC₆H₅, —COOC₆H₅, —SOCF₃, —SO₂CF₃, —SCF₃, —SO₂CN, —SCOCF₃, —SOR⁵, —S(OR⁵), —SC≡CR⁴—SO₂R⁵, —SSO₂R⁵, —SR⁵, —SSR⁴—SO₂CF₂CF₃, —SCF₂CF₃, —S(CF₃)═NSO₂CF₃, —SO₂C₆H₅, —SO₂N(R⁵)₂, —SO₂C(CF₃)₃, —SC(CF₃)₃, —SO(CF₃)═NSO₂CF₃, —S(O)(═NH)CF₃, —S(O)(═NH)R⁵, —S—C═CH₂, —SCOR⁵, —SOC₆H₅, —P(O)C₃F₇, —PO(OR⁵)₂, —PO(N(R⁵)₂)₂, —P(N(R⁵)₂)₂, —P(O)R⁵₂, and —PO(OR⁵)₂ and electron-withdrawing groups wherein the respective substituent can be in ortho, para or meta position in respect of the K (LG-O) group;

For the purposes of the present invention, the term “electron-drawing group” or “electron withdrawing group” refers to a chemical moiety (substituent) which is attached to the benzene ring, which is able to decrease the electron density of the benzene ring and which is listed in Chem. Rev. (1991), 91, 165-195, Table 1 (and references therein) with values of σₘ or σₚ>0;

at least one of —Y¹, —Y², —Y³, —Y⁴ and —Y⁵ are Further Substituents (-Q) which are independently from each other selected from the group comprising —H, —CN, -halogen, —CF₃, —NO₂, —COR⁵ and SO₂R⁵ wherein the respective substituent can be in ortho, para or meta position in respect of the K (LG-O) group;
wherein
   R⁴ is hydrogen or a linear or branched C₁-C₆ alkyl, more preferably hydrogen or linear or branched C₁-C₄ alkyl and most preferably hydrogen or methyl;
   R⁵ is hydrogen or a linear or branched C₁-C₆ alkyl, more preferably hydrogen or linear or branched C₁-C₄ alkyl and most preferably hydrogen or methyl;
      wherein further one of —Y¹, —Y², —Y³, —Y⁴ and Y⁵ is -A-B-D-P,
   wherein
   -A-B-D- is a bond or a spacer and
   P is a targeting agent.

The invention further refers to pharmaceutically acceptable salts or organic or inorganic acids, hydrates, esters, amides, solvates and prodrugs of the compounds having general chemical Formula A.

In a preferred embodiment, the targeting agent (P) is selected from peptides, peptidomimetics, small molecules or oligonucleotides.

Further, the First Substituent (-G) may also be selected from the group comprising —H and those members which have a value of the Hammet constant σ≧0.35 (compare Chem. Rev., (1991), 91, 165, Table 1) and which contains a fluoro or a nitrogen atom, namely: —F, —NO, —NO₂, —NR⁴SO₂CF₃, —N(CF₃)₂, —N(SO₂R⁵)₂, —N(O)═NCONH₂—N≡C, —N═NCF₃—N═NCN, —NR⁴COR⁴, —OSO₂CF₃, —OCOR⁵, —ONO₂, —OCF₂CF₃, —OCOCF₃, —OCN, —OCF₃, —C≡N, —C(NO₂)₃, —CONR⁴R⁵, —CH═NOR⁴, —COCF₃, —CF₃, —CF₂Cl—CBr₃ CClF₂₁—CF₂CF₃, —CH═NSO₂CF₃, —CH═NNHCSNH₂, —CF═CFCF₃, —CF₂—CF₂—CF₃, —CR⁴(CN)₂, —COCF₂CF₂CF₃, —C(CF₃)₃, —C(CN)₃, —CR⁴═C(CN)₂, —C(CN)═C(CN)₂, —SOCF₃, —SO₂CF₃, —SCF₃, —SO₂CN, —SCOCF₃, —SO₂CF₂CF₃, —SCF₂CF₃, —S(CF₃)═NSO₂CF₃, —SO₂N(R⁵)₂, —SO₂C(CF₃)₃, —SC(CF₃)₃, —SO(CF₃)—NSO₂CF₃, —S(O)(═NH)CF₃, —S(O)(═NH)R⁵ and —P(O)C₃F₇, wherein the respective substituent can be in ortho, para or meta position in respect of the K (LG-O) group. R⁴, R⁵ and R⁶ are used herein as given above.

Even more preferably the First Substituent (-G) may be selected from the group comprising —H or those members according to the preceding embodiment which have a value of the Hammet constant σ≧0.50 (compare Chem. Rev., (1991), 91, 165, Table 1) or which contains a fluoro atom, namely: —F, —NO, —NO₂, —NR⁴SO₂CF₃, —N(CF₃)₂, —N(O)═NCONH₂, —N═NCF₃, —N═NCN, —OSO₂CF₃, —ONO₂, OCF₂CF₃, —OCOCF₃, —OCN, —OCF₃, —C≡N, —C(NO₂)₃, —COCF₃, —CF₃₁—CF₂Cl—CBr₃, —CClF₂, —CF₂CF₃, —CH═NSO₂CF₃, —CF═CFCF₃, —CF₂—CF₂—CF₃, —CR⁴(CN)₃, —COCF₂CF₂CF₃, —C(CF₃)₃, —C(CN)₃, —CR⁴═C(CN)₂. —C(CN)═C(CN)₂, SOCF₃, —SO₂CF₃, —SCF₃, —SO₂CN, —SCOCF₃, —SO₂CF₂CF₃, —SCF₂CF₃, —S(CF₃)═NSO₂CF₃, —SO₂N(R⁵)₂, —SO₂C(CF₃)₃, —SC(CF₃)₃, —SO(CF₃)═NSO₂CF₃, —S(O)(═NH)CF₃ and —P(O)C₃F₇ wherein the respective substituent can be in ortho, para or meta position in respect of the K (LG-O) group and wherein R⁴ and R⁵ are used herein as given above.

Even more preferably the First Substitutent (-G) may be selected from the group comprising —H, —F, —NO₂, —OCF₂CF₃—OCF₃, —C≡N, —COCF₃—CF₃, —CF₂CF₃, —CF₂—CF₂—CF₃, —COCF₂CF₂CF₃, —SO₂CF₃, —SO₂CN, —SO₂CF₂CF₃, —SO₂N(R⁵)₂ and SC(CF₃)₃ wherein the respective substituent can be in ortho, para or meta position in respect of the K (LG-O) group and wherein R⁵ is used herein as given above.

In an alternative embodiment, the First Substituent (-G) may be selected from the group comprising —H and those members with a value of the Hammet constant σ≧0.50 (compare Chem. Rev., (1991), 91, 165, Table 1) or which contain a sulfur or a fluoro atom, namely: —F, —NR⁴SO₂CF₃, —N(CF₃)₂—N═NCF₃, —OSO₂CF₃—OCF₂CF₃, —OCOCF₃, —OCF₃, —COCF₃, —CF₃, —CF₂Cl—CBr₃, —CClF₂, —CF₂CF₃, —CH═NSO₂CF₃, —CF═CFCF₃, —CF₂—CF₂—CF₃, —COCF₂CF₂CF₃, —C(CF₃)₃, —SOCF₃, —SO₂CF₃, —SCF₃, —SO₂CN, —SO₂R⁵, —SCOCF₃, —SO₂CF₂CF₃, —SCF₂CF₃, —S(CF₃)═NSO₂CF₃, —SO₂N(R⁵)₂, —SO₂C(CF₃)₃, —SC(CF₃)₃, —SO(CF₃)═NSO₂CF₃, —S(O)(═NH)CF₃ and —P(O)C₃F₇ wherein the respective substituent can be in ortho, para or meta position in respect of the K (LG-O) group and wherein R⁴ and R⁵ are used herein as given above.

Even more preferably, the First Substituent (-G) may be selected from the group comprising —H, —F, —NR⁴SO₂CF₃, —OSO₂CF₃—OCF₂CF₃, —OCF₃, —COCF₃, —CF₃, —SO₂CF₃, SO₂R⁵ and —SO₂N(R⁵)₂ wherein the respective substituent can be in ortho, para or meta position in respect of the K (LG-O) group and wherein R⁴ and R⁵ are used herein as given above.

In an alternative embodiment, the First Substituent (-G) may be selected from the group comprising —H, —F, —Cl, —Br, —NO₂, —OSO₂R⁵, —OCF₃, —C≡N, —COOR⁴—CONR⁴R⁵, —COCF₃, —CF₂CF₃, —COR⁵, —CF₃, —C≡CF₃, —CF₂—CF₂—CF₃, —COC₆H₅, —SO₂CF₃, —SCOCF₃, —SO₂R⁵, —SO₂CF₂CF₃, —SO₂C₆H₅—SO₂N(R⁵)₂, and —PO(OR)₂ wherein the respective substituent can be in ortho, para or meta position in respect of the K (LG-O) group and wherein R⁴ and R⁵ are used herein as given above.

Even more preferably, the First Substituent (-G) may be selected from the group comprising —H, —F, —Cl, —Br, —NO₂, —NR⁴SO₂R⁵, —NR⁴COR⁴, —NR⁴COOR⁵, —C≡N, —CONR⁴R⁵, —C≡CR⁴, —COR⁵, —CF₃, and —SO₂R⁵ wherein the respective substituent can be in ortho, para or meta position in respect of the K (LG-O) group and wherein R⁴ and R⁵ are used herein as given above.

Even more preferably, the First Substituent (-G) may be selected from the group comprising —H, —F, —Cl, —Br, —NO₂, —C≡N, —CF₃, —SO₂CF₃, —SO₂R⁵, —SO₂C₆H₅ and —SO₂N(R⁵)₂ wherein the respective substituent can be in ortho, para or meta position in respect of the K (LG-O) group and wherein R⁴ and R⁵ are used herein as given above.

A positive value of a Hammet constant is a measure of electron deficiency. It seems that certain combinations of substituents with particular atoms (nitrogen, sulfur and/or fluoro) are favourable over others. For example nitrogen or fluoro substituents combined with positive Hammet constants allow a F-18 radiolabeling with relative high radiochemical yields whereas sulfur or fluoro atoms seem to guarantee radiolabeling reactions with only minor side reactions. It is for example known from literature that the choice of substituent can influence the ratio of ring fluorination versus methyl fluoride formation at trimethylammonium benzene derivatives with two substituents in total (review Coenen, “Fluorine-18 Labeling Methods: Features and Possibilities of Basic Reactions” (2006), in: P. A. Schubiger, M. Friebe, L. Lehmann, (eds), PET-Chemistry—The Driving Force in Molecular Imaging. Springer, Berlin Heidelberg, p. 15-50, in particular p. 23-26).

In a further embodiment of the invention, any of the Further Substituents (-Q) may independently from each other be selected from the group comprising —H, —CN, —F, —Cl, —Br and —NO₂, wherein the respective substituent can be in ortho, para or meta position in respect of the K (LG-O) group.

More preferably, any of the Further Substituents (-Q) may independently from each other be selected from the group comprising —H, —CN, —F and —NO₂ wherein the respective substituent can be in ortho, para or meta position in respect of the K (LG-O) group.

Most preferably, any of the Further Substituents (-Q) may independently from each other be selected from the group comprising —H, —CN or —F wherein the respective substituent can be in ortho, para or meta position in respect of the K (LG-O) group.

In a further preferred embodiment of the invention, any of the First Substituent —Y¹, —Y², —Y³, —Y⁴ and —Y⁵ defined by G and said Further Substituents Substituent —Y¹, —Y², —Y³, —Y⁴ and —Y⁵ defined by Q may independently from each other be selected from the group comprising —H, —CN, —F, —Cl, —CF₃, —NO₂, —COCH₃ and —SO₂CH₃ wherein the respective substituent can be in ortho, para or meta position in respect of the K (LG-O) group.

More preferably any of the First Substituent and said Further Substituents may independently from each other be selected from the group comprising —H, —CN and —Cl wherein the respective substituent can be in ortho, para or meta position in respect of the K (LG-O) group.

In a further embodiment of the invention —Y¹ may be selected from the group comprising —H, —F, —Cl, —Br, —I, —NO, —NO₂, —NR⁴COCF₃, —NR⁴SO₂CF₃, —N(CF₃)₂, —NHCSNHR⁵, —N(SO₂R⁶)₂, —N(O)—NCONH₂, —NR⁵CN, —NHCSR⁶, —N═C, —N═C(CF₃)₂, —N═NCF₃, —N═NCN, —NR⁵COR⁶, —NR⁵COOR⁶, —OSO₂CF₃, —OSO₂CO₆H₅, —OCOR⁶, —ONO₂, —OSO₂R⁶, —O—C≡CH₂, —OCF₂CF₃, —OCOCF₃, —OCN, —OCF₃, —C≡N, —C(NO₂)₃, —COOR⁵, —CONR⁵R⁶, —CSNH₂, —CH═NOR⁵, —CH₂SO₂R⁵, —COCF₃, —CF₃, —CF₂Cl—CBr₃, —CClF₂, —CCl₃, —CF₂CF₃, —C≡CR⁴, —CH═NSO₂CF₃, —CH₂CF₃, —COR⁶, —CH═NOR⁶, —CH₂CONH₂, —CSNHR⁶, —CH═NNHCSNH₂, —CH═NNHCONHNH₂—C≡CF₃, —CF═CFCF₃, —CF₂—CF₂—CF₃, —CR⁵(CN)₂, —COCF₂CF₂CF₃, —C(CF₃)₃, —C(CN)₃, —CR⁵═C(CN)₂, −1-pyrryl, —C(CN)═C(CN)₂, —C-pyridyl, —COC₆H₅, —COOC₆H₅, —SOCF₃, —SO₂CF₃, —SCF₃, —SO₂CN, —SCOCF₃, —SOR⁶, S(OR⁶), —SC≡CR⁵, —SO₂R⁶, —SSO₂R⁶, —SR⁶, —SSR⁶, —SO₂CF₂CF₃, —SCF₂CF₃, —S(CF₃)═NSO₂CF₃, —SO₂C₆H₅, SO₂N(R⁶)₂, —SO₂C(CF₃)₃, —SC(CF₃)₃, —SO(CF₃)═NSO₂CF₃, —S(O)═NCF₃, —S(O)═NR⁶, —S—C═CH₂, —SCOR⁵, —SOC₆H₅, —P(O)C₃F₇, —PO(R⁶)₂, —PO(N(R⁶)₂)₂, —P(N(R⁶)₂)₂, —P(O)(R⁶)₂, —PO(OR⁶)₂ and electron-withdrawing groups wherein the respective substituent can be in ortho, para or meta position in respect of the K (LG-O) group and

Y⁵ may be selected from the group comprising —CN, Cl, —F, —Br, —CF₃, —NO₂, —COR⁵ and —SO₂R⁵ wherein the respective substituent can be in ortho, para or meta position in respect of the K (LG-O) group.

Most preferably —Y¹ and —Y⁵ may independently from each other be selected from the group comprising —CN and —Cl and, more preferably, only one of —Y¹ and —Y⁵ may be —CN or —Cl and other group is —H. Thus, either one or both substituents which are in ortho position to —K at the benzene ring are —CN or —Cl.

In a further embodiment of the invention, the First Substitutent (-G) may be selected from the group comprising —H, —F, —Cl, —Br, —I, —NO, —NO₂, —NR⁴COCF₃, —NR⁴SO₂CF₃, —N(CF₃)₂, —NHCSNHR⁴, —N(SO₂R⁵)₂, —N(O)═NCONH₂, —NR⁴CN, —NHCSR⁵, —N≡C, —N═C(CF₃)₂, —N═NCF₃, —N═NCN, —NR⁴COR⁴, —NR⁴COOR⁵, —OSO₂CF₃, —OSO₂C₆H₅, —OCOR⁵, —ONO₂, —OSO₂R⁵, —O—C═CH₂, —OCF₂CF₃, —OCOCF₃, —OCN, —OCF₃, —C≡N, —C(NO₂)₃, —COOR⁴, —CONR⁴R⁵, —C(S)NH₂, —CH═NOR⁴, —CH₂SO₂R⁴—COCF₃, —CF₃, —CF₂Cl—CBr₃, —CClF₂, —CCl₃, —CF₂CF₃, —C≡CR⁴, —CH—NSO₂CF₃, —CH₂CF₃, —COR⁵, —CH═NOR⁵, —CH₂CONH₂, —CSNHR⁵, —CH═NNHCSNH₂, —CH═NNHCONHNH₂, —C≡CF₃, —CF═CFCF₃, —CF₂—CF₂—CF₃, —CR⁴(CN)₂, —COCF₂CF₂CF₃, —C(CF₃)₃, —C(CN)₃, —CR⁴═C(CN)₂, −1-pyrryl, —C(CN)═C(CN)₂, —C-pyridyl, —COC₆H₅, —COOC₆H₅, —SOCF₃, —SO₂CF₃, —SCF₃, —SO₂CN, —SCOCF₃, —SOR⁵, —S(OR⁵), —SC≡CR⁴, —SO₂R⁵, —SSO₂R⁵, —SR⁵, —SSR⁴—SO₂CF₂CF₃, —SCF₂CF₃, —S(CF₃)═NSO₂CF₃, —SO₂CO₆H₅, —SO₂N(R⁵)₂, —SO₂C(CF₃)₃, —SC(CF₃)₃, —SO(CF₃)═NSO₂CF₃, —S(O)(═NH)CF₃—S(O)(═NH)R⁵, —S—C═CH₂, —SCOR⁵, —SOC₆H₅, —P(O)C₃F₇, —PO(OR⁵)₂, —PO(N(R⁵)₂)₂, —P(N(R⁵)₂)₂, —P(O)R⁵)₂, and —PO(OR⁵)₂ wherein the respective substituent can be in ortho, para or meta position in respect of the K (LG-O) group or another electron-drawing group;

one of the Further Substitutents (-Q) is selected from the group comprising —H, —CN, halogen, —SO₂—R⁵ and —NO₂, wherein R⁵ is hydrogen or C₁-C₆ linear or branched alkyl, wherein the respective substituent can be in ortho, para or meta position in respect of the K (LG-O) group and
the other Further Substitutents (-Q) are hydrogen,
such that

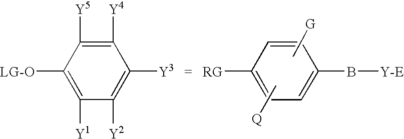

wherein RG-=LG-O— and —B—Y-E=-A-B-D-P, as one of —Y¹, —Y², —Y³, —Y⁴ and —Y⁵ is -A-B-D-P.

In all the above cases referring to the First Substituent (-G) and the Further Substituents (-Q) at least one thereof is not —H.

In a further embodiment of the present invention R⁴ may be hydrogen or linear or branched C₁-C₄ alkyl. Further, R⁵ may be hydrogen or linear or branched C₁-C₄ alkyl.

In a further embodiment of the present invention, G and Q may never be at the same time a —H.

In a preferred embodiment of compounds of Formula I, -G and -Q are independently from each other selected from —H, —CN, CF₃, and —Cl.

In a more preferred embodiment -G and -Q are independently from each other H, —CF₃, or CN.

In a even more preferred embodiment in a more preferred embodiment -G and -Q are independently from each other H, —CF₃, or —CN, whereas at least -G or -Q is —CF₃ or —CN.

In a further preferred embodiment -A- may preferably be selected from the group comprising a bond, —CO—, —SO₂—, —(CH₂)_{d}—CO—, —SO—, —C≡C—CO—, —[CH₂]ₘ-E-[CH₂]ₙ—CO—, —[CH₂]ₘ-E-[CH₂]ₙ—SO₂—, —C(═O)—O—, —NR¹⁰—, —O—, —(S)ₚ—, —C(O)NR¹²—, —NR¹²—, —C(═S)NR¹²—, —C(═S)O—, C₁-C₆ cycloalkyl, alkenyl, heterocycloalkyl, unsubstituted and substituted aryl, heteroaryl, aralkyl, heteroaralkyl, alkylenoxy, arylenoxy, aralkylenoxy, —SO₂NR¹³—, —NR¹³SO₂—, —NR¹³C(═O)O—, —NR¹³C(═O)NR²—, —NH—NH— and —NH—O—,
wherein
d is an integer of from 1 to 6,
m and n, independently, are any integer of from 0 to 5;
-E- is a bond, —S—, —O— or —NR⁹—,
wherein R⁹ is H, C₁-C₁₀ alkyl, aryl, heteroaryl or aralkyl,
p is any integer of from 1 to 3;
R¹⁰, R¹¹ and R¹², independently, are H, C₁-C₁₀ alkyl, aryl, heteroaryl or aralkyl and R¹³ is H, substituted or nonsubstituted, linear or branched C₁-C₆ alkyl, aryl, cycloalkyl, heterocycloalkyl, aryl, heteroaryl, aralkyl or heteroaralkyl.

More preferably, -A- may be selected from the group comprising —CO—, —SO₂— and —C≡C—CO—.

Most preferably -A- may be selected from the group comprising —CO— and —SO₂—.

—B— may preferably be —NH— or —NR′—,
wherein R′ is a branched, cyclic or linear C₁-C₆ alkyl group.

The C₁-C₆ alkyl group may be preferably a CH₃ or C₂H₅.

—B— may be preferably —NH— or —NCH₃
-D- may preferably be —(CH₂)ₚ—CO— wherein p being an integer of from 1 to 10 or —(CH₂—CH₂—O)_{q}—CH₂—CH₂—CO— with q being an integer of from 1 to 5.

Alternatively, the moiety —B-D- together may form a bond, be one amino acid residue, an amino acid sequence with two (2) to twenty (20) amino acid residues or a non-amino acid group.

-B-D- may preferably be an amino acid sequence with two (2) to twenty (20) amino acid residues. More preferably the amino acid sequence may comprise a natural or unnatural amino acid sequence or mixture thereof.

Even more preferably —B-D- may be Arg-Ser, Arg-Ava, Lys(Me)2-β-ala, Lys(Me)2-ser, Arg-β-ala, Ser-Ser, Ser-Thr, Arg-Thr, S-alkylcysteine, Cysteic acid, thioalkylcysteine (S—S-Alkyl) or

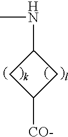

wherein k and l are independently selected in the range of from 0 to 4.

Even more preferably —B-D- may be a non-amino acid moiety selected from the group comprising

—NH—(CH₂)ₚ—CO—, wherein p is an integer of from 1 to 10,
—NH—(CH₂—CH₂—O)_{q}—CH₂—CH₂—CO—, wherein q is an integer of from 1 to 5,
—NH-cycloalkyl-CO— wherein cycloalkyl is selected from C₅-C₈ cycloalkyl, more preferably C₆ atom cycloalkyl, and
—NH-heterocycloalkyl-(CH₂)ᵥ—CO— wherein heterocycloalkyl is selected from C₅-C₈ heterocycloalkyl containing carbon atoms and 1, 2, 3 or 4 oxygen, nitrogen or sulfur heteroatoms more preferably 1 to 2 heteroatom even more preferably 1 heteroatom and V is an integer of from 1 to 41 more preferably v is an integer of from 1 to 2.

In a highly preferable embodiment of the present invention each one of —Y¹, —Y², —Y³, —Y⁴ and —Y⁵ may independently from each other be —H, —CN, —Cl, —F, —CF₃, —NO₂, —COCH₃ or —SO₂CH₃, more preferably H, CN and Cl, and most preferably Y¹ and Y₅ may independently from each other be CN or Cl or either Y₁ or Y₅ may be CN or Cl, with the proviso that exactly one residue of —Y¹, —Y², —Y³, —Y⁴ and —Y⁵ is A-B-D-P, wherein
-A- is —CO— or —SO₂—, more preferably —CO—,
further either:
—B— is —NH— or —NR′—, wherein R′ is a branched, cyclic or linear C₁ to C₆ alkyl group, preferably CH₃ or C₂H₅, most preferably B is NH or NCH₃,
-D- is —(CH₂)ₚ—CO— with p being an integer of from 1 to 10, more preferably —(CH₂)₄—CO—, or -D- is —(CH₂—CH₂-0)_{q}—CH₂—CH₂—CO— with q being an integer of from 1 to 5,
or:
—B-D- together is a bond or one amino acid residue or an amino acid sequence with two (2) to twenty (20) amino acid residues,
P is a targeting agent and
LG is a leaving group, suitable for displacement by means of a nucleophilic aromatic substitution reaction.

P is a targeting agent.

For the purposes of the present invention, the term “targeting agent” shall have the following meaning: The targeting agent is a compound or moiety that targets or directs the radionuclide attached to it to a specific site in a biological system. A targeting agent can be any compound or chemical entity that binds to or accumulates at a target site in a mammalian body, i.e., the compound localizes to a greater extent at the target site than to surrounding tissue.

The compounds of this invention are useful for the imaging of a variety of cancers including but not limited to: carcinoma such as bladder, breast, colon, kidney, liver, lung, including small cell lung cancer, esophagus, gall-bladder, ovary, pancreas, stomach, cervix, thyroid, prostate and skin, hematopoetic tumors of lymphoid and myeloid lineage, tumors of mesenchymal origin, tumors of central peripheral nervous systems, other tumors, including melanoma, seminoma, teratocarcinoma, osteosarcoma, xeroderma pigmentosum, keratoxanthoma, thyroid follicular cancer and Karposi's sarcoma. Most preferably, the use is not only for imaging of tumors, but also for imaging of inflammatory and/or neurodegenerative diseases, such as multiple sclerosis or Alzheimer's disease, or imaging of angiogenesis-associated diseases, such as growth of solid tumors, and rheumatoid arthritis.

Preferably the targeting agent is a peptide or a peptidomimetic or an oligonucleotide, particularly one which has specificity to target the complex to a specific site in a biological system. Small molecules effective for targeting certain sites in a biological system can also be used as the targeting agent.

Small molecules may be “small chemical entities”. As used in this application, the term “small chemical entity” shall have the following meaning: A small chemical entity is a compound that has a molecular mass of from 150 to 700, more preferably from 200 to 700, more preferably from 250 to 700, even more preferably from 300 to 700, even more preferably from 350 to 700 and most preferably from 400 to 700. A small chemical entity as used herein may further contain at least one aromatic or heteroaromatic ring and may also have a primary or secondary amine, a thiol or hydroxyl group coupled via which the benzene ring structure in the compounds of general chemical Formulae I and II is coupled via -A-B-D-. Such targeting moieties are known in the art, so are methods for preparing them.

The small molecule targeting agents may preferably be selected from those described in the following references: P. L. Jager, M, A. Korte, M. N. Lub-de Hooge, A. van Waarde, K. P. Koopmans, P. J, Perik and E. G. E. de Vries, Cancer Imaging, (2005) 5, 2732; W. D. Heiss and K. Herholz, J. Nucl. Med., (2006) 47(2), 302-312; and T. Higuchi and M. Schwaiger, Curr. Cardiol. Rep., (2006) 8(2), 131-138. More specifically examples of small molecule targeting agents are listed hereinafter:

| Name | Abbr. | target |
|---|---|---|
| 18F-2b-Carbomethoxy-3b-(4- | CFT | DAT (dopamine transporter) |
| fluorophenyl)tropane | | |
| 18F-Fluoroethylspiperone | FESP | D2 (dopamine 2 receptor), 5- |
| | | HT₂ |
| | | (5-hydroxytryptamine receptor) |
| 18F-Fallypride | | D2 (dopamine 2 receptor) |
| 18F-Altanserin | | 5-HT2A receptor |
| 18F-Cyclofoxy | | Opioid receptors |
| 18F-CPFPX | | Adenosine A1 receptor |
| Batimastat | | MMP |
| Fatty acids and analogues | | |
| Choline analogues | | |
| (metabolism) | | |
| Flumazenil | | Benzodiazepine receptors |
| Raclopride | | D2 receptors |
| Dihydrotestosteron and | | AR |
| analogues | | |
| Tamoxifen and analogues | | |
| Deoxyglucose | | |
| Thymidine | | Proliferation marker-thymidine |
| | | kinase |
| DOPA | | |
| benzazepines | | D₁ antagonists |
| N-methyl spiperone and | | dopamine receptors |
| derivatives thereof | | |
| benzamide raclopride; | | D₂ receptors |
| benzamide derivatives, e.g., | | |
| fallopride, iodo benzamide; | | |
| clozapine, quietapine | | |
| nomifensine, substituted | | DAT |
| analogs of cocaine, e.g., | | |
| tropane type derivatives of | | |
| cocaine, methyl phenidate | | |
| 2β-carboxymethoxy-3β-(4- | CIT | DAT |
| iodophenyl)tropane | | |
| | CIT-FE, CIT-FM | DAT |
| altanserin, setoperon, | | 5-HT_{2A} |
| ketanserin | | |
| | McN5652, 403U76 derivative | 5-HTT |
| | ADAM, DASP, MADAM | |
| acetylcholine analogues | MP3A, MP4A, PMP; QNB, | acetylcholine receptors |
| | TKB, NMPB, | |
| scopolamine, benztropine | | acetylcholine receptors |
| flumazenil | | GABA receptor |
| | RO-15-4513, FDG | GABA receptor |
| | PK-11195 | benzodiazepine receptor |
| xanthine analogues | CPFPX, MPDX | adenosine receptor |
| carfentanyl, diprenorphine | | opoid receptor |

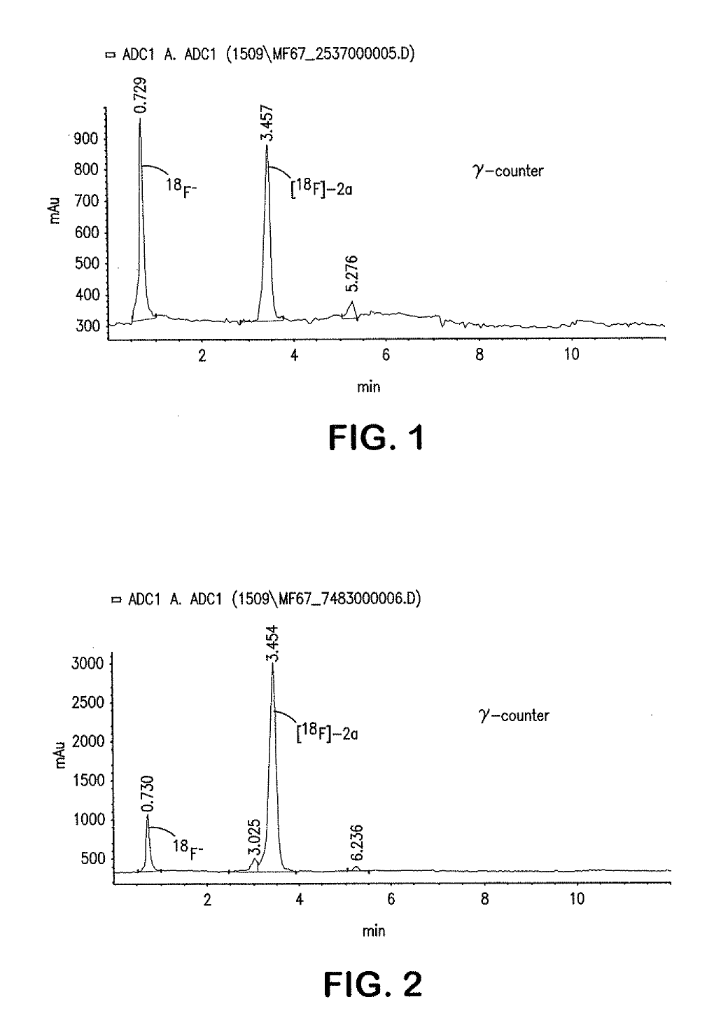

Further various smal molecule targeting agents and the targets thereof are given in Table 1 in W. D. Heiss and K. Herhoiz, ibid. and in FIG. 1 in T. Higuchi, M. Schwaiger, ibid.

Further preferred biomolecules are sugars, oligosaccharides, polysaccharides, aminoacids, nucleic acids, nucleotides, nucleosides, oligonucleotides, proteins, peptides, peptidomimetics, antibodies, aptamers, lipids, hormones (steroid and nonsteroid), neurotransmitters, drugs (synthetic or natural), receptor agonists and antagonists, dendrimers, fullerenes, virus particles and other targeting molecules/biomolecules (e.g., cancer targeting molecules).

P may be a peptide comprising from 4 to 100 amino acids wherein the amino acids may be selected from natural and non-natural amino acids and also may comprise modified natural and non-natural amino acids.

Examples for peptides as targeting agent (P) are, but are not limited to but are not limited to, somatostatin and derivatives thereof and related peptides, somatostatin receptor specific peptides, neuropeptide Y and derivatives thereof and related peptides, neuropeptide Y₁ and the analogs thereof, bombesin and derivatives thereof and related peptides, gastrin, gastrin releasing peptide and the derivatives thereof and related peptides, epidermal growth factor (EGF of various origin), insulin growth factor (IGF) and IGF-1, integrins (α₃β₁, αᵥβ₃, αᵥβ₅, αIIb₃), LHRH agonists and antagonists, transforming growth factors, particularly TGF-α; angiotensin; cholecystokinin receptor peptides, cholecystokinin (CCK) and the analogs thereof; neurotensin and the analogs thereof, thyrotropin releasing hormone, pituitary adenylate cyclase activating peptide (PACAP) and the related peptides thereof, chemokines, substrates and inhibitors for cell surface matrix metalloproteinase, prolactin and the analogs thereof, tumor necrosis factor, interleukins (IL-1, IL-2, IL-4 or IL-6), interferons, vasoactive intestinal peptide (VIP) and the related peptides thereof.

More preferably targeting agent (P) may be selected from the group comprising bombesin, somatostatin, neuropeptide Y₁, vasoactive intestinal peptide (VIP). Even more preferably targeting agent (P) may be selected from the group comprising bombesin, somatostatin, neuropeptide Y₁ and the analogs thereof. Even more preferably targeting agent (P) may be bombesin and derivatives, and related peptides thereof and the analogs thereof.

Bombesin is a fourteen amino acid peptide that is an analog of human Gastrin releasing peptide (GRP) that binds with high specificity to human GRP receptors present in prostate tumor, breast tumor and metastasis, In a more preferred embodiment, bombesin analogs have the following sequence having Formula III:

AA₁-AA₂-AA₃-AA₄-AA₅-AA₆-AA₇-AA₈-NT₁T₂ (type A) Formula III,
with:
T₁=T₂=H or T₁=H, T₂═OH or T₁ CH₃, T₂═OH
AA₁=Gln, Asn, Phe(4-CO—NH₂)
AA₂=Trp, D-Trp
AA₃=Ala, Ser, Val
AA₄=Val, Ser. Thr
AA₅=Gly, (N-Me)Gly
AA₆=His, His(3-Me), (N-Me)His, (N-Me)His(3-Me)
AA₇=Sta, Statine analogs and isomers, 4-Am, 5-MeHpA, 4-Am, 5-MeHxA, γ-substituted aminoacids
AA₈=Leu, Cpa, Cba, CpnA, Cha, t-bugly, tBuAla, Met, Nie, iso-Bu-Gly

In a more preferred embodiment, bombesin analogs have the following sequence of formula IV:

AA₁-AA₂-AA₃-AA₄-AA₅-AA₆-AA₇-AA₈-NT₁T₂ (type B) Formula IV,
with:
T₁=T₂=H or T₁=H, T₂═OH or T₁=CH₃, T₂═OH
AA₁=Gln, Asn or Phe(4-CO—NH₂)
AA₂=Trp, D-Trp
AA₃=Ala, Ser, Val
AA₄=Val, Ser. Thr
AA₅=βAla, β² and β³-amino acids as shown herein after

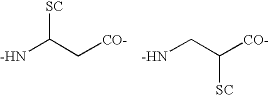

wherein SC represents a side chain found in proteinogenic amino acids and homologs of proteinogenic amino acids,
AA₆=His, His(3-Me), (N-Me)His, (N-Me)His(3-Me)
AA₇=Phe, Tha, NaI,
AA₈=Leu, Cpa, Cba, CpnA, Cha, t-buGly, tBuAla, Met, Nle, iso-Bu-Gly.

Therefore, in an even more preferred embodiment of the present invention targeting agent (P) may be selected from the group comprising bombesin analogs having sequence III or IV.

In a more preferred embodiment, bombesin analogs have the following sequences:

| | |
|---|---|
| Seq ID | P |

| | |
|---|---|
| Seq ID 1 | Gln-Trp-Ala-Val-NMeGly-His-Sta-Leu-NH₂ |
| Seq ID 2 | Gln-Trp-Ala-Val-Gly-His(Me)-Sta-Leu-NH₂ |
| Seq ID 3 | Gln-Trp-Ala-Val-NMeGly-His(3Me)-Sta-Leu-NH₂ |
| Seq ID 4 | Gln-Trp-Ala-Val-Gly-His(3Me)-Sta-Leu-NH₂ |
| Seq ID 7 | Gln-Trp-Ala-Val-NMeGly-His(3Me)-Sta-Cpa-NH₂ |
| Seq ID 8 | Gln-Trp-Ala-Val-Gly-His(3Me)-4-Am,5-MeHpA-Leu-NH₂ |
| Seq ID 12 | Gln-Trp-Ala-Val-Gly-His(3Me)-4-Am,5-MeHpA-Leu-NH₂ |
| Seq ID 17 | Gln-Trp-Ala--Val-Gly-His-4-Am,5-MeHpA-Leu-NH₂ |
| Seq ID 23 | Gln-Trp-Ala-Val-NMeGly-His(3Me)-4-Am,5-MeHpA-Cpa-NH₂ |
| Seq ID 27 | Gln-Trp-Ala-Val-NMeGly-His-FA02010-Cpa-NH₂ |
| Seq ID 28 | Gln-Trp-Ala-Val-NMeGly-His-4-Am,5-MeHpA-tbuGly-NH₂ |
| Seq ID 30 | Gln-Trp-Ala-Val-NMeGly-His(3Me)-Sta-tBuGly-NH₂ |
| Seq ID 32 | Gln-Trp-Ala-Val-NMeGly-His(3Me)-4-Am,5-MeHpA-Leu-NH₂ |
| Seq ID 33 | Gln-DTrp-Ala-Val-Gly-His-4-Am,5-MeHpA-tbuGly-NH₂ |
| Seq ID 34 | Gln-DTrp-Ala-Val-Gly-His-4-Am-5-MeHxA-Cpa-NH₂ |
| Seq ID 35 | Gln-Trp-Ala-Val-NMeGly-His(3Me)-Sta-Cpa-NH₂ |
| Seq ID 36 | Gln-DTrp-Ala-Val-Gly-His-Sta-tbuAla-NH₂ |
| Seq ID 42 | Gln-Trp-Ala-Val-Gly-His(3Me)-Sta-Cpa-NH₂ |
| Seq ID 43 | Gln-Trp-Ala-Val-Gly-His(3Me)-Sta-tBuGly-NH₂ |
| Seq ID 46 | Gln-Trp-Ala-Val-Gly-His(3Me)-4-Am,5-MeHpA-Leu-NH₂ |
| Seq ID 48 | Gln-Trp-Ala-Val-Gly-His(3Me)-4-Am,5-MeHpA-Leu-NH₂ |
| Seq ID 49 | Gln-Trp-Ala-Val-Gly-NMeHis-4-Am,5-MeHpA-Cpa-NH₂ |
| Seq ID 49 | Gln-Trp-Ala-Val-Gly-NMeHis(3Me)-4-Am,5-MeHpA-Leu-NH₂ |
| Seq ID 50 | Gln-Trp-Ala-Val-Gly-NMeHis-4-Am,5-MeHpA-Leu-NH₂ |
| Seq ID 51 | Gln-Trp-Ala-Val-NMeGly-His-AHMHxA-Leu-NH₂ |
| Seq ID 52 | Gln-Trp-Ala-Val-βAla-NMeHis-Tha-Cpa-NH₂ |
| Seq ID 53 | Gln-Trp-Ala-Val-βAla-NMeHis-Phe-Cpa-NH₂ |
| Seq ID 54 | Gln-Trp-Ala-Val-βAla-NMeHis-Phe-Leu-NH₂ |
| Seq ID 55 | Gln-Trp-Ala-Val-βAla-DHis-Phe-Leu-NH₂ |
| Seq ID 56 | Gln-Trp-Ala-Val-βAla-His-βhLeu-Leu-NH₂ |
| Seq ID 57 | Gln-Trp-Ala-Val-βAla-His-βhIle-Leu-NH₂ |
| Seq ID 58 | Gln-Trp-Ala-Val-βAla-His-βhLeu-tbuGly-NH₂ |
| Seq ID 59 | Gln-Trp-Ala-Val-βAla-His(3Me)-Phe-Tha-NH₂ |
| Seq ID 60 | Gln-Trp-Ala-Val-βAla-His(3Me)-Phe-Nle-NH₂ |
| Seq ID 61 | Gln-Trp-Ala-Val-βAla-NMeHis-Phe-tbuGly-NH₂ |
| Seq ID 62 | Gln-Trp-Ala-Val-βAla-NMeHis-Tha-tbuGly-NH₂ |
| Seq ID 63 | Gln-Trp-Ala-Val-βAla-His(3Me)-Tha-tbuGly-NH₂ |
| Seq ID 64 | Gln-Trp-Ala-Val-βAla-His(3Me)-Phe-Cpa-NH₂ |
| Seq ID 65 | Gln-Trp-Ala-NMeVal-βAla-His-Phe-Leu-NH₂ |
| Seq ID 66 | Gln-Trp-Ala-Val-βAla-His-NMePhe-Leu-NH₂ |
| Seq ID 67 | Gln-DTrp-Ala-Val-βAla-His-Phe-Leu-NH₂ |
| Seq ID 68 | Gln-Trp-DAla-Val-βAla-His-Phe-Leu-NH₂ |
| Seq ID 69 | Gln-Trp-Ala-DVal-βAla-His-Phe-Leu-NH₂ |
| Seq ID 70 | Gln-Trp-Ala-Val-βAla-His-DPhe-Leu-NH₂ |
| Seq ID 71 | Gln-Trp-Ala-Val-βAla-His-βhIle-tbuGly-NH₂ |
| Seq ID 72 | Gln-Trp-Ala-Val-NMeGly-His-4-Am,5-MeHpA-Cpa-NH₂ |
| Seq ID 73 | Gln-Trp-Ala-Val-NMeGly-His-Sta-Cpa-NH₂ |
| Seq ID 74 | Gln-Trp-Ala-Val-NMeGly-His-Sta-tbuAla-NH₂ |
| Seq ID 75 | Gln-Trp-Ala-Val-NMeGly-His-4-Am,5-MeHpA-tbuAla-NH₂ |
| Seq ID 77 | Gln-Trp-Ala-Val-His(Me)-Sta-Leu-NH₂ |
| Seq ID 82 | Gln-Trp-Ala-Val-Gly-His(3Me)-FA4-Am,5-MeHpA-Leu-NH₂ |
| Seq ID 90 | Gln-Trp-Ala-Val-Gly-His(3Me)-4-Am,5-MeHpA-Leu-NH₂ |
| Seq ID 91 | Gln-Trp-Ala-Val-Gly-His-4-Am,5-MeHpA-Leu-NH₂ |
| Seq ID 101 | Gln-Trp-Ala-Val-Gly-His(3Me)-4-Am-5-MeHpA-4-amino-5- |
| | methylheptanoic acid-Leu-NH₂ |
| Seq ID 102 | Gln-Trp-Ala-Val-NMeGly-His(3Me)-4-Am-5-MeHpA-4-amino-5- |
| | methylheptanoic acid-Cpa-NH₂ |

Thus, the invention also refers to bombesin analogs that bind specifically to human GRP receptors present in prostate tumor, breast tumor and metastasis. In a preferred embodiment, the bombesin analogs are peptides having sequences from Seq ID 1 to Seq ID 102 and preferably have one of them. More preferably a bombesin analog is additionally labeled with a fluorine isotope (F) wherein fluorine isotope (F) is selected from ¹⁸F or ¹⁹F. More preferably the bombesin analog is radiolabeled with ¹⁸F. The bombesin analog is preferably radiolabeled using the radiofluorination method of the present invention.

In a more preferred embodiment, somatostatin analogs have the following sequences:

| | |
|---|---|
| Seq ID 104 | ----c[Lys-(NMe)Phe-1Nal-D-Trp-Lys-Thr] |
| Seq ID 105 | ----c[Dpr-Met-(NMe)Phe-Tyr-D-Trp-Lys] |

In a more preferred embodiment, neuropeptide Y₁ analogs have the following sequences:

| | |
|---|---|
| Seq ID 106 | -DCys-Leu-Ile-Thr-Arg-Cys-Arg-Tyr-NH₂ |
| Seq ID 107 | -DCys-Leu-Ile-Val-Arg-Cys-Arg-Tyr-NH₂ |

(_indicates disulfide bridge)

In a more preferred embodiment, peptide is tetrapeptide of the following sequences:

valyl-β-alanyl-phenylalanyl-glycine amide
valyl-β-alanyl-histidyl(π-Me)-glycine amide

In a further preferred embodiment of the present invention, the targeting agent P may comprise a combination of any of the aforementioned bioactive molecules suitable to bind to a target site together with a reacting moiety which serves the linking between the bioactive molecule and the rest of the compound of the invention (Formulae I, II, III), wherein reacting moiety is selected from —NR⁴, —NR⁴—(CH₂)ₙ—, —O—(CH₂)ₙ— or —S—(CH₂)ₙ—, wherein R⁴ is hydrogen or alkyl and n is an integer from 1 to 6 and wherein the suitable bioactive molecule is selected from peptide, peptidomimetic, oligonucleotide, or small molecule.

In a preferred embodiment P is NR⁷-peptide, or —(CH₂)ₙ-peptide, —O—(CH₂)ₙ— peptide or —S—(CH₂)ₙ— peptide, NR⁷— small-molecule, or —(CH₂)ₙ— small-molecule, —O—(CH₂)ₙ— small-molecule or —S—(CH₂)ₙ— small-molecule, NR⁷— oligonucleotide, or —(CH₂)ₙ— oligonucleotide, —O—(CH₂)ₙ— oligonucleotide or —S—(CH₂)ₙ— oligonucleotide, wherein n is an integer of from 1 to 6.

In a more preferred embodiment P is —NR⁴-peptide, —(CH₂)ₙ-peptide, wherein n is an integer of from 1 to 6.

In another more preferred embodiment P is —NR⁴-oligonucleotide or —(CH₂)ₙ— oligonucleotide, wherein n is an integer of from 1 to 6.

In another more preferred embodiment P is —NR⁴-small-molecule or —(CH₂)ₙ-small molecule, wherein n is an integer of from 1 to 6.

In a preferred embodiment, the precursor (Formula I) for a single step radiolabeling method may be the following precursor bombesin analog:

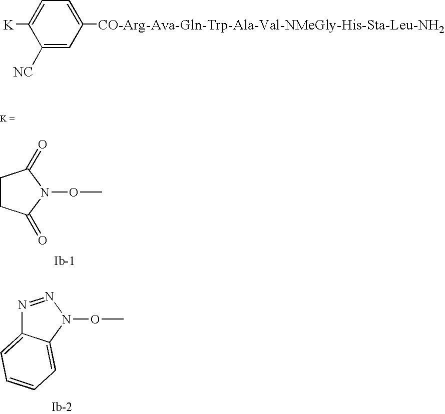

In a preferred embodiment, the precursor (Formula I) is one of the following precursor peptide analog:

4-(Benzotriazol-1-yloxy)-3-cyano-benzoyl-valyl-β-alanyl-phenylalanyl-glycine amide,
4-(Benzotriazol-1-yloxy)-3-cyano-benzoyl-valyl-β-alanyl-histidyl(TT-Me)-glycine amide,
3-cyano-4-([1,2,3]triazolo[4,5-b]pyridin-3-yloxy)-benzoyl-(5-aminopentanoyl)-
phenylalanyl-(4(S)-amino-3(S)-hydroxy-6-methyl)heptanoyl-leucine amide,
4-(benzotriazol-1-yloxy)-3-chloro-benzoyl-Valyl-β-alanyl-phenylalanyl-glycine amide,
4-(Benzotriazol-1-yloxy)-3-chloro-benzoyl-Arg-Ava-Gln-Trp-Ala-Val-NMeGly-His(3Me)-
Sta-Leu-NH₂,
4-(Benzotriazol-1-yloxy)-3-cyano-benzoyl-1,4-cis-Achc-Gln-Trp-Ala-Val-Gly-His(3Me)-
Sta-Leu-NH₂,
4-(Benzotriazol-1-yloxy)-3-chloro-benzoyl-Gln-Trp-Ala-Val-Gly-His(3Me)-Sta-Leu-NH₂,
4-(Benzotriazol-1-yloxy)-3-chloro-benzoyl-AOC-Gln-Trp-Ala-Gly-His(3Me)-Sta-Leu-
NH₂,
4-(Benzotriazol-1-yloxy)-3-cyano-benzoyl-Ava-Gln-Trp-Ala-Val-NMeGly-His(3Me)-Sta-
Cpa-NH₂,
4-(Benzotriazol-1-yloxy)-3-cyano-benzoyl-Ava-Gln-Trp-Ala-Val-Gly-His(3Me)-FA4-
Am,5-MeHpA-Leu-NH₂,
3-Cyano-4-(2,5-dioxo-pyrrolidin-1-yloxy)-benzoyl-Ava-Gln-Trp-Ala-Val-Gly-His(3Me)-
Sta-Leu-NH₂,
3-Cyano-4-(2,5-dioxo-pyrrolidin-1-yloxy)-benzoyl-Arg-Ava-Gln-Trp-Ala-Val-Gly-
His(3Me)-Sta-Leu-NH₂,
3-Chloro-4-(2,5-dioxo-pyrrolidin-1-yloxy)-benzoyl-Arg-Ava-Gln-Trp-Ala-Val-Gly-
His(3Me)-Sta-Leu-NH₂,
3-Chloro-4-(2,5-dioxo-pyrrolidin-1-yloxy)-benzoyl-Ava-Gln-Trp-Ala-Val-Gly-His(3Me)-
Sta-Leu-NH₂,

In a further preferred embodiment targeting agent (P) may be selected from the group comprising oligonucleotides comprising from 4 to 100 nucleotides.

Preferred oligonucleotide is TTA1 (see experimental part).

In a preferred embodiment, the precursor (Formula I) is one of the following precursor with small molecule:

### 3-Cyano-4-(2,5-dioxo-pyrrolidin-1-yloxy)-N-(thymidinyl-propyl)-benzamide

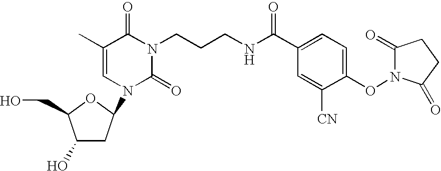

### 3-Cyano-4-(benzotriazol-1-yloxy)-N-(thymidinyl-propyl)-benzamide

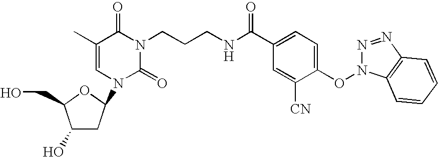

In preferred embodiments of compounds having general chemical Formula I, the leaving group LG is selected from the group comprising

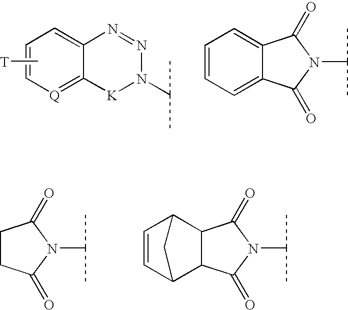

wherein,
T is H or Cl,
Q is CH or N,
K is absent or is C═O

In a more preferred embodiment LG is selected from the group comprising

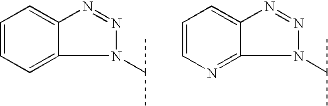

The compound according to Formula I serves as precursor of the compound according to Formula II, wherein the leaving group LG-O is replaced in a labeling reaction with a fluorine isotope, more preferably with a ¹⁸F or ¹⁹F even more preferably with a ¹⁸F.

In a second aspect the present invention refers to compounds having general chemical Formula II,

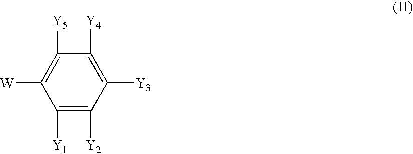

wherein the residues and substituents —Y¹, —Y², —Y³, —Y⁴ and —Y⁵ have the same meaning as depicted above for compounds having general chemical Formula I. This includes in particular all preferred embodiments mentioned above with regard to the residues and substituents —Y¹, —Y², —Y³, Y⁴ and —Y⁵, -A-, —B—, -D-, and —P
and to pharmaceutically acceptable salts, inorganic or organic acids, hydrates, esters, amides, solvates and prodrugs thereof.

W is a fluorine isotope (F) selected from radioactive or non-radioactive isotope of fluorine. The radioactive fluorine isotope is selected from ¹⁸F. The non-radioactive “cold” fluorine isotope is selected from ¹⁹F.

If W is preferably ¹⁸F, the compound of the invention having general chemical Formula II being radio pharmaceutically labelled with ¹⁸F has the following general chemical Formula IIA:

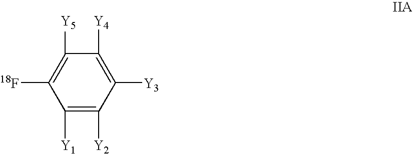

Most preferably, when W=¹⁹F then the compound having general chemical Formula II has the general chemical Formula IIB:

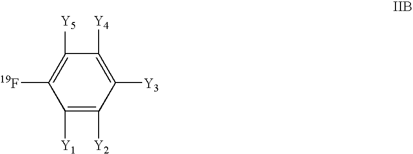

in a preferred embodiment of compounds of Formula II, —Y¹, Y², Y³, Y⁴ and —Y⁵ are independently from each other selected from —H, —CN and —Cl.

In a more preferred embodiment —Y¹, —Y², —Y³, —Y⁴ and —Y⁵ are independently from each other CN or Cl.

In a preferred embodiment, the compound of formula II labelled with ¹⁸F or ¹⁹F is selected from the following list, wherein targeting agent (P) is selected from peptide, peptidomimetic, smaller organic molecule or oligonucleotide and all preferred form disclosed above.

More preferably the targeting agent (P) of compound of formula II is a bombesin analog:

| | |
|---|---|
| IIA-a-1 | 4-[18]Fluoro-3-cyano-benzoyl-Arg-Ava-Gln-Trp-Ala-Val-NMeGly-His-Sta- |
| | Leu-NH₂, |
| IIA-a-2 | 4-[18]Fluoro-3-cyano-benzoyl-Arg-Ava-Gln-Trp-Ala-Val-Gly-His(Me)-Sta- |
| | Leu-NH₂, |
| IIA-a-3 | 4-[18]Fluoro-3-cyano-benzoyl-Arg-Ava-Gln-Trp-Ala-Val-NMeGly-His(3Me)- |
| | Sta-Leu-NH₂, |
| IIA-a-4 | 4-[18]Fluoro-3-cyano-benzoyl-1,4-cis-Achc-Gln-Trp-Ala-Val-Gly-His(3Me)- |
| | Sta-Leu-NH₂, |
| IIA-a-5 | 4-[18]Fluoro-3-cyano-benzoyl-Gln-Trp-Ala-Val-Gly-His(3Me)-Sta-Leu-NH₂, |
| IIA-a-6 | 4-[18]Fluoro-3-cyano-benzoyl-AOC-Gln-Trp-Ala-Val-Gly-His(3Me)-Sta-Leu- |
| | NH₂, |
| IIA-a-7 | 4-[18]Fluoro-3-cyano-benzoyl-Ava-Gln-Trp-Ala-Val-NMeGly-His(3Me)-Sta- |
| | Cpa-NH₂, |
| IIA-a-8 | 4-[18]Fluoro-3-cyano-benzoyl-Ava-Gln-Trp-Ala-Val-Gly-His(3Me)-FA4- |
| | Am,5-MeHpA-Leu-NH₂, |
| IIA-a-9 | 4-[18]Fluoro-3-cyano-benzoyl-Ava-Gln-Trp-Ala-Val-Gly-His(3Me)-Sta-Leu- |
| | NH₂, |
| IIA-a-10 | 4-[18]Fluoro-3-cyano-benzoyl-Lys(Me)2-βAla-Gln-Trp-Ala-Val-Gly- |
| | His(3Me)-Sta-Leu-NH₂, |
| IIA-a-11 | 4-[18]Fluoro-3-cyano-benzoyl-Lys(Me)2-βAla-Gln-Trp-Ala-Val-Gly- |
| | His(3Me)-Sta-Leu-NH₂, |
| IIA-a-12 | 4-[18]Fluoro-3-cyano-benzoyl-Arg-Ser-Gln-Trp-Ala-Val-Gly-His(3Me)-4- |
| | Am,5-MeHpA-Leu-NH₂, |
| IIA-a-13 | 4-[18]Fluoro-3-cyano-benzoyl-Ser-Ser-Gln-Trp-Ala-Val-Gly-His(3Me)-4- |
| | Am,5-MeHpA-Leu-NH₂, |
| IIA-a-14 | 4-[18]Fluoro-3-cyano-benzoyl-Lys(Me)2-Ser-Gln-Trp-Ala-Val-Gly-His(3Me)- |
| | 4-Am,5-MeHpA-Leu-NH₂, |
| IIA-a--15 | 4-[18]Fluoro-3-cyano-benzoyl-Arg-Ser-Gln-Trp-Ala-Val-Gly-His(3Me)-Sta- |
| | Leu-NH₂, |
| IIA-a-16 | 4-[18]Fluoro-3-cyano-benzoyl-Lys(Me)2-βAla-Gln-Trp-Ala-Val-Gly- |
| | His(3Me)-4-Am,5-MeHpA-Leu-NH₂, |
| IIA-a-17 | 4-[18]Fluoro-3-cyano-benzoyl-Ava-Gln-Trp-Ala-Val-Gly-His-4-Am,5- |
| | MeHpA-Leu-NH₂, |
| IIA-a-18 | 4-[18]Fluoro-3-trifluoromethyl-benzoyl-Arg-Ava-Gln-Trp-Ala-Val-Gly- |
| | His(3Me)-Sta-LeuNH₂, |
| IIA-a-19 | 4-[18]Fluoro-3-trifluoromethyl-benzoyl-Arg-Ava-Gln-Trp-Ala-Val-NMeGly- |
| | His(3Me)-Sta-Leu-NH₂, |
| IIA-a-20 | 4-[18]Fluoro-3-trifluoromethyl-benzoyl-1,4-cis-Achc-Gln-Trp-Ala-Val-Gly- |
| | His(3Me)-Sta-Leu-NH₂, |
| IIA-a-21 | 4-[18]Fluoro-3-trifluoromethyl-benzoyl-Gln-Trp-Ala-Val-Gly-His(3Me)-Sta- |
| | Leu-NH₂, |
| IIA-a-22 | 4-[18]Fluoro-3-trifluoromethyl-benzoyl-Arg-βAla-Gln-Trp-Ala-Val-Gly- |
| | His(3Me)-4-Am,5-MeHpA-Leu-NH₂, |
| IIB-a-23 | 4-[18]-Fluoro-3-cyano-benzoyl-Ava-Gln-Trp-Ala-Val-NMeGly-His(3Me)-4- |
| | Am,5-MeHpA-Cpa-NH₂, |
| IIB-a-24 | 4-[18]-Fluoro-3-cyano-benzoyl-Ser-Ser-Gln-Trp-Ala-Val-Gly-His(3Me)-Sta- |
| | Leu-NH₂, |
| IIB-a-25 | 4-[18]-Fluoro-3-cyano-benzoyl-DOA-Gln-Trp-Ala-Val-Gly-His(3Me)-Sta- |
| | Leu-NH₂, |
| IIB-a-26 | 3,4-[18]-Difluorobenzoyl-Ava-Gln-Trp-Ala-Val-NMeGly-His-Sta-Leu-NH₂, |
| IIB-a-27 | 3,4-[18]-Difluorobenzoyl-Ava-Gln-Trp-Ala-Val-NMeGly-His-FA02010-Cpa- |
| | NH₂, |
| IIB-a-28 | 3,4-[18]-Difluorobenzoyl-Ava-Gln-Trp-Ala-Val-NMeGly-His-4-Am,5-MeHpA- |
| | tbuGly-NH₂, |
| IIB-a-29 | 3,4-[18]-Difluorobenzoyl-Ava-Gln-Trp-Ala-Val-NMeGly-His(3Me)-Sta-Leu- |
| | NH₂, |
| IIB-a-30 | 3,4-[18]-Difluorobenzoyl-Ava-Gln-Trp-Ala-Val-NMeGly-His(3Me)-Sta- |
| | tBuGly-NH₂, |
| IIB-a-31 | 3,4-[18]-Difluorobenzoyl-Ava-Gln-Trp-Ala-Val-Gly-His(3Me)-Sta-Leu-NH₂, |
| IIB-a-32 | 3,4-[18]-Difluorobenzoyl-Ava-Gln-Trp-Ala-Val-NMeGly-His(3Me)-4-Am,5- |
| | MeHpA-Leu-NH₂, |
| IIB-a-33 | 3,4-[18]-Difluorobenzoyl-Ava-Gln-DTrp-Ala-Val-Gly-His-4-Am,5-MeHpA- |
| | tbuGly-NH₂, |
| IIB-a-34 | 3,4-[18]-Difluorobenzoyl-Ava-Gln-DTrp-Ala-Val-Gly-His-4-Am-5-MeHxA- |
| | Cpa-NH₂, |
| IIB-a-35 | 3,4-[18]-Difluorobenzoyl-Ava-Gln-Trp-Ala-Val-NMeGly-His(3Me)-Sta-Cpa- |
| | NH₂, |
| IIB-a-36 | 3,4-[18]-Difluorobenzoyl-Ava-Gln-DTrp-Ala-Val-Gly-His-Sta-tbuAla-NH₂, |
| IIB-a-37 | 3,4-[18]-Difluorobenzoyl-Arg-Ava-Gln-Trp-Ala-Val-NMeGly-His-Sta-Leu- |
| | NH₂, |
| IIB-a-38 | 3,4-[18]-Difluorobenzoyl-Gln-Trp-Ala-Val-Gly-His(3Me)-Sta-Leu-NH₂, |
| IIB-a-39 | 3,4-[18]-Difluorobenzoyl-Arg-Ava-Gln-Trp-Ala-Val-NMeGly-His(3Me)-Sta- |
| | Leu-NH₂, |
| IIB-a-40 | 3,4-[18]-Difluorobenzoyl-Arg-Ava-Gln-Trp-Ala-Val-Gly-His(3Me)-Sta-Leu- |
| | NH₂, |
| IIB-a-41 | 3,4-[18]-Difluorobenzoyl-Arg-βAla-Arg-Gln-Trp-Ala-Val-Gly-His(3Me)-Sta- |
| | Leu-NH₂, |
| IIB-a-42 | 3,4-[18]-Difluorobenzoyl-Ava-Gln-Trp-Ala-Val-Gly-His(3Me)-Sta-Cpa-NH₂, |
| IIB-a-43 | 3,4-[18]-Difluorobenzoyl-Ava-Gln-Trp-Ala-Val-Gly-His(3Me)-Sta-tBuGly- |
| | NH₂, |
| IIB-a-44 | 3,4-[18]-Difluorobenzoyl-Arg-Arg-Gln-Trp-Ala-Val-NMeGly-His(3Me)-Sta- |
| | Leu-NH₂, |
| IIB-a-45 | 3,4-[18]-Difluorobenzoyl-Arg-βAla-Gln-Trp-Ala-Val-NMeGly-His(3Me)-Sta- |
| | Leu-NH₂, |
| IIB-a-46 | 3,4-[18]-Difluorobenzoyl-Gln-Trp-Ala-Val-Gly-His(3Me)-4-Am,5-MeHpA- |
| | Leu-NH₂, |
| IIB-a-47 | 3,4-[18]-Difluorobenzoyl-Ava-Gln-Trp-Ala-Val-NMeGly-His(3Me)-4-Am,5- |
| | MeHpA-Cpa-NH₂, |
| IIB-a-48 | 3,4-[18]-Difluorobenzoyl-Ava-Gln-Trp-Ala-Val-Gly-His(3Me)-4-Am,5- |
| | MeHpA-Leu-NH₂, |
| IIB-a-49 | 3,4-[18]-Difluorobenzoyl-Ava-Gln-Trp-Ala-Val-Gly-NMeHis-4-Am,5-MeHpA- |
| | Cpa-NH₂, |
| IIB-a-49 | 3,4-[18]-Difluorobenzoyl-Ava-Gln-Trp-Ala-Val-Gly-NMeHis(3Me)-4-Am,5- |
| | MeHpA-Leu-NH₂, |
| IIB-a-50 | 3,4-[18]-Difluorobenzoyl-Ava-Gln-Trp-Ala-Val-Gly-NMeHis-4-Am,5-MeHpA- |
| | Leu-NH₂, |
| IIB-a-51 | 3,4-[18]-Difluorobenzoyl-Ava-Gln-Trp-Ala-Val-NMeGly-His-AHMHxA-Leu- |
| | NH₂, |
| IIB-a-52 | 3,4-[18]-Difluorobenzoyl-Ava-Gln-Trp-Ala-Val-βAla-NMeHis-Tha-CPa-NH₂, |
| IIB-a-53 | 3,4-[18]-Difluorobenzoyl-Ava-Gln-Trp-Ala-Val-βAla-NMeHis-Phe-CPa-NH₂, |
| IIB-a-54 | 3,4-[18]-Difluorobenzoyl-Ava-Gln-Trp-Ala-Val-βAla-NMeHis-Phe-Leu-NH₂, |
| IIB-a-55 | 3,4-[18]-Difluorobenzoyl-Ava-Gln-Trp-Ala-Val-βAla-DHis-Phe-Leu-NH₂, |
| IIB-a-56 | 3,4-[18]-Difluorobenzoyl-Ava-Gln-Trp-Ala-Val-βAla-His-βhLeu-Leu-NH₂, |
| IIB-a-57 | 3,4-[18]-Difluorobenzoyl-Ava-Gln-Trp-Ala-Val-βAla-His-βhIle-Leu-NH₂, |
| IIB-a-58 | 3,4-[18]-Difluorobenzoyl-Ava-Gln-Trp-Ala-Val-βAla-His-βhLeu-tbuGly-NH₂, |
| IIB-a-59 | 3,4-[18]-Difluorobenzoyl-Ava-Gln-Trp-Ala-Val-βAla-His(3Me)-Phe-Tha-NH₂, |
| IIB-a-60 | 3,4-[18]-Difluorobenzoyl-Ava-Gln-Trp-Ala-Val-βAla-His(3Me)-Phe-Nle-NH₂, |
| IIB-a-61 | 3,4-[18]-Difluorobenzoyl-Ava-Gln-Trp-Ala-Val-βAla-NMeHis-Phe-tbuGly- |
| | NH₂, |
| IIB-a-62 | 3,4-[18]-Difluorobenzoyl-Ava-Gln-Trp-Ala-Val-βAla-NMeHis-Tha-tbuGly- |
| | NH₂, |
| IIB-a-63 | 3,4-[18]-Difluorobenzoyl-Ava-Gln-Trp-Ala-Val-βAla-His(3Me)-Tha-tbuGly- |
| | NH₂, |
| IIB-a-64 | 3,4-[18]-Difluorobenzoyl-Ava-Gln-Trp-Ala-Val-βAla-His(3Me)-Phe-Cpa-NH₂, |
| IIB-a-65 | 3,4-[18]-Difluorobenzoyl-Ava-Gln-Trp-Ala-NMeVal-βAla-His-Phe-Leu-NH₂, |
| IIB-a-66 | 3,4-[18]-Difluorobenzoyl-Ava-Gln-Trp-Ala-Val-βAla-His-NMePhe-Leu-NH₂, |
| IIB-a-67 | 3,4-[18]-Difluorobenzoyl-Ava-Gln-DTrp-Ala-Val-βAla-His-Phe-Leu-NH₂, |
| IIB-a-68 | 3,4-[18]-Difluorobenzoyl-Ava-Gln-Trp-DAla-Val-βAla-His-Phe-Leu-NH₂, |
| IIB-a-69 | 3,4-[18]-Difluorobenzoyl-Ava-Gln-Trp-Ala-DVal-βAla-His-Phe-Leu-NH₂, |
| IIB-a-70 | 3,4-[18]-Difluorobenzoyl-Ava-Gln-Trp-Ala-Val-βAla-His-DPhe-Leu-NH₂, |
| IIB-a-71 | 3,4-[18]-Difluorobenzoyl-Ava-Gln-Trp-Ala-Val-βAla-His-βhIle-tbuGly-NH₂, |
| IIB-a-72 | 4-[18]-Fluoro-3-cyano-phenylsulfonyl-Ava-Gln-Trp-Ala-Val-NMeGly-His-4- |
| | Am,5-MeHpA-Cpa-NH₂, |
| IIB-a-73 | 4-[18]-Fluoro-3-cyano-phenylsulfonyl-Ava-Gln-Trp-Ala-Val-NMeGly-His- |
| | Sta-Cpa-NH₂, |
| IIB-a-74 | 4-[18]-Fluoro-3-cyano-phenylsulfonyl-Ava-Gln-Trp-Ala-Val-NMeGly-His- |
| | Sta-tbuAla-NH₂, |
| IIB-a-75 | 4-[18]-Fluoro-3-cyano-phenylsulfonyl-Ava-Gln-Trp-Ala-Val-NMeGly-His-4- |
| | Am,5-MeHpA-tbuAla-NH₂, |
| | 4-[18]Fluoro-3-cyano-benzoyl-(piperidyl-4-carbonyl)-Gln-Trp-Ala-Val-Gly-His(3Me)-Sta- |
| | Leu-NH₂, |
| | 4-[18]Fluoro-3-cyano-benzoyl-(piperazin-1-yl-acetyl)-Gln-Trp-Ala-Val-Gly-His(3Me)- |
| | Sta-Leu-NH₂, |
| | 4-[18]Fluoro-3-cyano-benzoyl-1,4-trans-Achc-Gln-Trp-Ala-Val-NMeGly-His-Sta-Leu- |
| | NH₂, |
| IIB-a-1 | 4-[19]-Fluoro-3-cyano-benzoyl-Arg-Ava-Gln-Trp-Ala-Val-NMeGly-His-Sta- |
| | Leu-NH₂, |
| IIB-a--2 | 4-[19]-Fluoro-3-cyano-benzoyl-Arg-Ava-Gln-Trp-Ala-Val-His(Me)-Sta-Leu- |
| | NH₂, |
| IIB-a-3 | 4-[19]-Fluoro-3-cyano-benzoyl-Arg-Ava-Gln-Trp-Ala-Val-NMeGly-His(3Me)- |
| | Sta-Leu-NH₂, |
| IIB-a-4 | 4-[19]-Fluoro-3-cyano-benzoyl-1,4-cis-Achc-Gln-Trp-Ala-Val-Gly-His(3Me)- |
| | Sta-Leu-NH₂, |
| IIB-a-5 | 4-[19]-Fluoro-3-cyano-benzoyl-Gln-Trp-Ala-Val-Gly-His(3Me)-Sta-Leu-NH₂, |
| IIB-a-6 | 4-[19]-Fluoro-3-cyano-benzoyl-AOC-Gln-Trp-Ala-Val-Gly-His(3Me)-Sta- |
| | Leu-NH₂, |
| IIB-a-7 | 4-[19]-Fluoro-3-cyano-benzoyl-Ava-Gln-Trp-Ala-Val-NMeGly-His(3Me)-Sta- |
| | Cpa-NH₂, |
| IIB-a-8 | 4-[19]-Fluoro-3-cyano-benzoyl-Ava-Gln-Trp-Ala-Val-Gly-His(3Me)-4-Am,5- |
| | MeHpA-Leu-NH₂, |
| IIB-a-9 | 4-[19]-Fluoro-3-cyano-benzoyl-Ava-Gln-Trp-Ala-Val-Gly-His(3Me)-Sta-Leu- |
| | NH₂, |
| IIB-a-10 | 4-[19]-Fluoro-3-cyano-benzoyl-Lys(Me)2-βAla-Gln-Trp-Ala-Val-Gly- |
| | His(3Me)-Sta-Leu-NH₂, |
| IIB-a-11 | 4-[19]-Fluoro-3-cyano-benzoyl-Lys(Me)2-βAla-Gln-Trp-Ala-Val-Gly- |
| | His(3Me)-Sta-Leu-NH₂, |
| IIB-a-12 | 4-[19]-Fluoro-3-cyano-benzoyl-Arg-Ser-Gln-Trp-Ala-Val-Gly-His(3Me)-4- |
| | Am,5-MeHpA-Leu-NH₂, |
| IIB-a-13 | 4-[19]-Fluoro-3-cyano-benzoyl-Ser-Ser-Gln-Trp-Ala-Val-Gly-His(3Me)-4- |
| | Am,5-MeHpA-Leu-NH₂, |
| IIB-a-14 | 4-[19]-Fluoro-3-cyano-benzoyl-Lys(Me)2-Ser-Gln-Trp-Ala-Val-Gly- |
| | His(3Me)-4-Am,5-MeHpA-Leu-NH₂, |
| IIB-a-15 | 4-[19]-Fluoro-3-cyano-benzoyl-Arg-Ser-Gln-Trp-Ala-Val-Gly-His(3Me)-Sta- |
| | Leu-NH₂, |
| IIB-a-16 | 4-[19]-Fluoro-3-cyano-benzoyl-Lys(Me)2-βAla-Gln-Trp-Ala-Val-Gly- |
| | His(3Me)-4-Am,5-MeHpA-Leu-NH₂, |
| IIB-a-17 | 4-[19]-Fluoro-3-cyano-benzoyl-Ava-Gln-Trp-Ala-Val-Gly-His-4-Am,5- |
| | MeHpA-Leu-NH₂, |
| IIB-a-18 | 4-[19]-Fluoro-3-trifluoromethyl-benzoyl-Arg-Ava-Gln-Trp-Ala-Val-Gly- |
| | His(3Me)-Sta-Leu-NH₂, |
| IIB-a-19 | 4-[19]-Fluoro-3-trifluoromethyl-benzoyl-Arg-Ava-Gln-Trp-Ala-Val-NMeGly- |
| | His(3Me)-Sta-Leu-NH₂, |
| IIB-a-20 | 4-[19]-Fluoro-3-trifluoromethyl-benzoyl-1,4-cis-Achc-Gln-Trp-Ala-Val-Gly- |
| | His(3Me)-Sta-Leu-NH₂, |
| IIB-a-21 | 4-[19]-Fluoro-3-trifluoromethyl-benzoyl-Gln-Trp-Ala-Val-Gly-His(3Me)-Sta- |
| | Leu-NH₂, |
| IIB-a-22 | 4-[19]-Fluoro-3-trifluoromethyl-benzoyl-Arg-βAla-Gln-Trp-Ala-Val-Gly- |
| | His(3Me)-4-Am,5-MeHpA-Leu-NH₂, |
| IIB-a-23 | 4-[19]-Fluoro-3-cyano-benzoly-Ava-Gln-Trp-Ala-Val-NMeGly-His(3Me)-4- |
| | Am,5-MeHpA-Cpa-NH₂, |
| IIB-a-24 | 4-[19]-Fluoro-3-cyano-benzoyl-Ser-Ser-Gln-Trp-Ala-Val-Gly-His(3Me)-Sta- |
| | Leu-NH₂, |
| IIB-a-25 | 4-[19]-Fluoro-3-cyano-benzoly-DOA-Gln-Trp-Ala-Val-Gly-His(3Me)-Sta- |
| | Leu-NH₂, |
| IIB-a-26 | 3,4-[19]-Difluorobenzoyl-Ava-Gln-Trp-Ala-Val-NMeGly-His-Sta-Leu-NH₂, |
| IIB-a-27 | 3,4-[19]-Difluorobenzoyl-Ava-Gln-Trp-Ala-Val-NMeGly-His-FA02010-Cpa- |
| | NH₂, |
| IIB-a-28 | 3,4-[19]-Difluorobenzoyl-Ava-Gln-Trp-Ala-Val-NMeGly-His-4-Am,5-MeHpA- |
| | tbuGly-NH₂, |
| IIB-a-29 | 3,4-[19]-Difluorobenzoyl-Ava-Gln-Trp-Ala-Val-NMeGly-His(3Me)-Sta-Leu- |
| | NH₂, |
| IIB-a-30 | 3,4-[19]-Difluorobenzoyl-Ava-Gln-Trp-Ala-Val-NMeGly-His(3Me)-Sta- |
| | tBuGly-NH₂, |
| IIB-a-31 | 3,4-[19]-Difluorobenzoyl-Ava-Gln-Trp-Ala-Val-Gly-His(3Me)-Sta-Leu-NH₂, |
| IIB-a-32 | 3,4-[19]-Difluorobenzoyl-Ava-Gln-Trp-Ala-Val-NMeGly-His(3Me)-4-Am,5- |
| | MeHpA-Leu-NH₂, |
| IIB-a-33 | 3,4-[19]-Difluorobenzoyl-Ava-Gln-DTrp-Ala-Val-Gly-His-4-Am,5-MeHpA- |
| | tbuGly-NH₂, |
| IIB-a-34 | 3,4-[19]-Difluorobenzoyl-Ava-Gln-DTrp-Ala-Val-Gly-His-4-Am-5-MeHxA- |
| | Cpa-NH₂, |
| IIB-a-35 | 3,4-[19]-Difluorobenzoyl-Ava-Gln-Trp-Ala-Val-NMeGly-His(3Me)-Sta-Cpa- |
| | NH₂, |
| IIB-a-36 | 3,4-[19]-Difluorobenzoyl-Ava-Gln-DTrp-Ala-Val-Gly-His-Sta-tbuAla-NH₂, |
| IIB-a-37 | 3,4-[19]-Difluorobenzoyl-Arg-Ava-Gln-Trp-Ala-Val-NMeGly-His-Sta-Leu- |
| | NH₂, |
| IIB-a-38 | 3,4-[19]-Difluorobenzoyl-Gln-Trp-Ala-Val-Gly-His(3Me)-Sta-Leu-NH₂, |
| IIB-a-39 | 3,4-[19]-Difluorobenzoyl-Arg-Ava-Gln-Trp-Ala-Val-NMeGly-His(3Me)-Sta- |
| | Leu-NH₂, |
| IIB-a-40 | 3,4-[19]-Difluorobenzoyl-Arg-Ava-Gln-Trp-Ala-Val-Gly-His(3Me)-Sta-Leu- |
| | NH₂, |
| IIB-a-41 | 3,4-[19]-Difluorobenzoyl-Arg-βAla-Arg-Gln-Trp-Ala-Val-Gly-His(3Me)-Sta- |
| | Leu-NH₂, |
| IIB-a-42 | 3,4-[19]-Difluorobenzoyl-Ava-Gln-Trp-Ala-Val-Gly-His(3Me)-Sta-Cpa-NH₂, |
| IIB-a-43 | 3,4-[19]-Difluorobenzoyl-Ava-Gln-Trp-Ala-Val-Gly-His(3Me)-Sta-tBuGly- |
| | NH₂, |
| IIB-a-44 | 3,4-[19]-Difluorobenzoyl-Arg-Arg-Gln-Trp-Ala-Val-NMeGly-His(3Me)-Sta- |
| | Leu-NH₂, |
| IIB-a-45 | 3,4-[19]-Difluorobenzoyl-Arg-βAla-Gln-Trp-Ala-Val-NMeGly-His(3Me)-Sta- |
| | Leu-NH₂, |
| IIB-a-46 | 3,4-[19]-Difluorobenzoyl-Gln-Trp-Ala-Val-Gly-His(3Me)-4-Am,5-MeHpA- |
| | Leu-NH₂, |
| IIB-a-47 | 3,4-[19]-Difluorobenzoyl-Ava-Gln-Trp-Ala-Val-NMeGly-His(3Me)-4-Am,5- |
| | MeHpA-Cpa-NH₂, |
| IIB-a-48 | 3,4-[19]-Difluorobenzoyl-Ava-Gln-Trp-Ala-Val-Gly-His(3Me)-4-Am,5- |
| | MeHpA-Leu-NH₂, |
| IIB-a-49 | 3,4-[19]-Difluorobenzoyl-Ava-Gln-Trp-Ala-Val-Gly-NMeHis-4-Am,5-MeHpA- |
| | Cpa-NH₂, |
| IIB-a-49 | 3,4-[19]-Difluorobenzoyl-Ava-Gln-Trp-Ala-Val-Gly-NMeHis(3Me)-4-Am,5- |
| | MeHpA-Leu-NH₂, |
| IIB-a-50 | 3,4-[19]-Difluorobenzoyl-Ava-Gln-Trp-Ala-Val-Gly-NMeHis-4-Am,5-MeHpA- |
| | Leu-NH₂, |
| IIB-a-51 | 3,4-[19]-Difluorobenzoyl-Ava-Gln-Trp-Ala-Val-NMeGly-His-AHMHxA-Leu- |
| | NH₂, |
| IIB-a-52 | 3,4-[19]-Difluorobenzoyl-Ava-Gln-Trp-Ala-Val-βAla-NMeHis-Tha-Cpa-NH₂, |
| IIB-a-53 | 3,4-[19]-Difluorobenzoyl-Ava-Gln-Trp-Ala-Val-βAla-NMeHis-Phe-Cpa-NH₂, |
| IIB-a-54 | 3,4-[19]-Difluorobenzoyl-Ava-Gln-Trp-Ala-Val-βAla-NMeHis-Phe-Leu-NH₂, |
| IIB-a-55 | 3,4-[19]-Difluorobenzoyl-Ava-Gln-Trp-Ala-Val-βAla-DHis-Phe-Leu-NH₂, |
| IIB-a-56 | 3,4-[19]-Difluorobenzoyl-Ava-Gln-Trp-Ala-Val-βAla-His-βhLeu-Leu-NH₂, |
| IIB-a-57 | 3,4-[19]-Difluorobenzoyl-Ava-Gln-Trp-Ala-Val-βAla-His-βhIle-Leu-NH₂, |
| IIB-a-58 | 3,4-[19]-Difluorobenzoyl-Ava-Gln-Trp-Ala-Val-βAla-His-βhLeu-tbuGly-NH₂, |
| IIB-a-59 | 3,4-[19]-Difluorobenzoyl-Ava-Gln-Trp-Ala-Val-βAla-His(3Me)-Phe-Tha-NH₂, |
| IIB-a-60 | 3,4-[19]-Difluorobenzoyl-Ava-Gln-Trp-Ala-Val-βAla-His(3Me)-Phe-Nle-NH₂, |
| IIB-a-61 | 3,4-[19]-Difluorobenzoyl-Ava-Gln-Trp-Ala-Val-βAla-NMeHis-Phe-tbuGly- |
| | NH₂, |
| IIB-a-62 | 3,4-[19]-Difluorobenzoyl-Ava-Gln-Trp-Ala-Val-βAla-NMeHis-Tha-tbuGly- |
| | NH₂, |
| IIB-a-63 | 3,4-[19]-Difluorobenzoyl-Ava-Gln-Trp-Ala-Val-βAla-His(3Me)-Tha-tbuGly- |
| | NH₂, |
| IIB-a-64 | 3,4-[19]-Difluorobenzoyl-Ava-Gln-Trp-Ala-Val-βAla-His(3Me)-Phe-Cpa-NH₂, |
| IIB-a-65 | 3,4-[19]-Difluorobenzoyl-Ava-Gln-Trp-Ala-NMeVal-βAla-His-Phe-Leu-NH₂, |
| IIB-a-66 | 3,4-[19]-Difluorobenzoyl-Ava-Gln-Trp-Ala-Val-βAla-His-NMePhe-Leu-NH₂, |
| IIB-a-67 | 3,4-[19]-Difluorobenzoyl-Ava-Gln-DTrp-Ala-Val-βAla-His-Phe-Leu-NH₂, |
| IIB-a-68 | 3,4-[19]-Difluorobenzoyl-Ava-Gln-Trp-DAla-Val-βAla-His-Phe-Leu-NH₂, |
| IIB-a-69 | 3,4-[19]-Difluorobenzoyl-Ava-Gln-Trp-Ala-DVal-βAla-His-Phe-Leu-NH₂, |
| IIB-a-70 | 3,4-[19]-Difluorobenzoyl-Ava-Gln-Trp-Ala-Val-βAla-His-DPhe-Leu-NH₂, |
| IIB-a-71 | 3,4-[19]-Difluorobenzoyl-Ava-Gln-Trp-Ala-Val-βAla-His-βhIle-tbuGly-NH₂, |
| IIB-a-72 | 4-[19]-Fluoro-3-cyano-phenylsulfonyl-Ava-Gln-Trp-Ala-Val-NMeGly-His-4- |
| | Am,5-MeHpA-Cpa-NH₂, |
| IIB-a-73 | 4-[19]-Fluoro-3-cyano-phenylsulfonyl-Ava-Gln-Trp-Ala-Val-NMeGly-His- |
| | Sta-Cpa-NH₂, |
| IIB-a-74 | 4-[19]-Fluoro-3-cyano-phenylsulfonyl-Ava-Gln-Trp-Ala-Val-NMeGly-His- |
| | Sta-tbuAla-NH₂, |
| IIB-a-75 | 4-[19]-Fluoro-3-cyano-phenylsulfonyl-Ava-Gln-Trp-Ala-Val-NMeGly-His-4- |
| | Am,5-MeHpA-rbuAla-NH₂, |
| | 4-[19]-Fluoro-3-cyano-benzoyl-(piperidyl-4-carbonyl)-Gln-Trp-Ala-Val-Gly-His(3Me)- |
| | Sta-Leu-NH₂, |
| | 4-[19]-Fluoro-3-cyano-benzoyl-(piperazin-1-yl-acetyl)-Gln-Trp-Ala-Val-Gly-His(3Me)- |
| | Sta-Leu-NH₂, |
| | 4-[19]-Fluoro-3-cyano-benzoyl-1,4-trans-Achc-Gln-Trp-Ala-Val-NMeGly-His-Sta-Leu- |
| | NH₂, |

In a preferred embodiment, the radiopharmaceutical labeled with ¹⁸F or ¹⁹F is selected from the following list, wherein targeting agent (P) is a somatostatin analog:

| | |
|---|---|
| IIA-a-76: | 4-[18]Fluoro-3-cyano-benzoyl-Ava-ε-c[Lys- |
| | (NMe)Phe-1Nal-D-Trp-Lys-Thr] |
| IIA-a-77: | 4-[18]Fluoro-3-cyano-benzoyl-Ava-β-c[Dpr- |
| | Met-(NMe)Phe-Tyr-D-Trp-Lys] |
| IIB-a-76: | 4-[19]Fluoro-3-cyano-benzoyl-Ava-ε-c[Lys- |
| | (NMe)Phe-1Nal-D-Trp-Lys-Thr] |
| IIB-a-77: | 4-[19]Fluoro-3-cyano-benzoyl-Ava-β-c[Dpr- |
| | Met-(NMe)Phe-Tyr-D-Trp-Lys] |

In a preferred embodiment, the radiopharmaceutical labelled with ¹⁸F or ¹⁹F is selected from the following list, wherein targeting agent (P) is a neuropeptide Y₁ analog;

| | |
|---|---|
| IIA-a-78: | 4-[18]Fluoro-3-cyano-benzoyl-Ava-DCys- |
| | Leu-Ile-Thr-Arg-Cys-Arg-Tyr-NH₂ |
| IIA-a-79: | 4-[18]Fluoro-3-cyano-benzoyl-Ava-DCys- |
| | Leu-Ile-Val-Arg-Cys-Arg-Tyr-NH₂ |
| IIA-a-78: | 4-[19]Fluoro-3-cyano-benzoyl-Ava-DCys- |
| | Leu-Ile-Thr-Arg-Cys-Arg-Tyr-NH₂ |
| IIA-a-79: | 4-[19]Fluaro-3-cyano-benzoyl-Ava-DCys- |
| | Leu-Ile-Val-Arg-Cys-Arg-Tyr-NH₂ |

In a preferred embodiment, the radiopharmaceutical labelled with ¹⁸F or ¹⁹F is selected from the following list, wherein targeting agent (P) is a tetrapeptide:
3-cyano-4-fluoro-benzoyl-valyl-β-alanyl-phenylalanyl-glycine amide [¹⁹F],
3-cyano-4-fluoro-benzoyl-valyl-β-alanyl-phenylalanyl-glycine amide [¹⁸F],
3-cyano-4-fluoro-benzoyl-valyl-β-alanyl-histidyl(π-Me)-glycine amide [¹⁹F],
3-cyano-4-fluoro-benzoyl-valyl-β-alanyl-histidyl(π-Me)-glycine amide [¹⁸F],
3-cyano-4-fluoro-benzoyl-(5-aminopentanoyl)-phenylalanyl-(4(S)-amino-3(S)-hydroxy-6-methyl)heptanoyl-leucine amide [¹⁹F],
3-cyano-4-fluoro-benzoyl-(5-aminopentanoyl)-phenylalanyl-(4(S)-amino-3(S)-hydroxy-6-methyl)heptanoyl-leucine amide [¹⁸F],

In a preferred embodiment, the radiopharmaceutical labelled with ¹⁸F or ¹⁹F is selected from the following list, wherein targeting agent (P) is a small molecule:

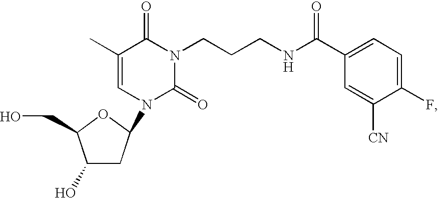

3-Cyano-4-[F-19]fluoro-N-(thymidinyl-propyl)-benzamide,
3-Cyano-4-[F-18]fluoro-N-(thymidinyl-propyl)-benzamide;

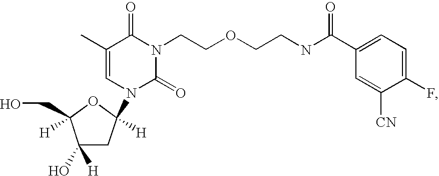

3-Cyano-4-[F-18]fluoro-N-(2-[2-thymidinyl-ethoxy]-ethyl)-benzamide,
3-Cyano-4-[F-18]fluoro-N-(2-[2-thymidinyl-ethoxy]-ethyl)-benzamide;

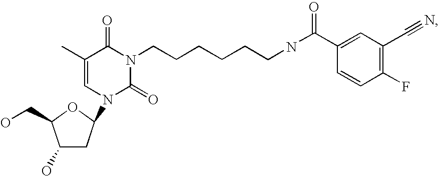

3-Cyano-4-[F-19]fluoro-N-(thymidinyl-hexyl)-benzamide,
3-Cyano-4-[F-18]-fluoro-N-(thymidinyl-hexyl)-benzamide;

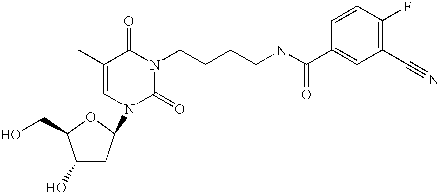

3-Cyano-4-[19F]fluoro-N-(thymidinyl-butyl)benzamide,
3-Cyano-4-[18F]fluoro-N-(thymidinyl-butyl)benzamide;

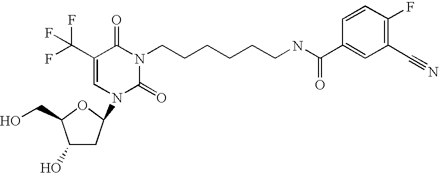

wherein F is ¹⁸F or ¹⁹F,
3-Cyano-4-fluoro-N-(trifluoromethyl thymidinyl-hexyl)benzamide,
3-Cyano-4-fluoro-N-(trifluoromethyl thymidinyl-hexyl)benzamide;

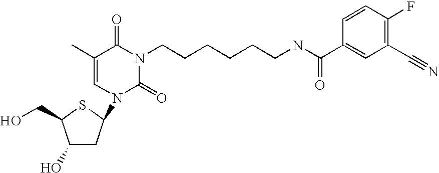

wherein F is ¹⁸F or ¹⁹F,
3-Cyano-4-fluoro[F-18]-N-{6-[3-((2R,4S,5R)-4-hydroxy-5-hydroxymethyl-tetrahydro-thiophen-2-yl)-5-methyl-2,6,dioxo-3,6-dihydro-2H-pyrimidin-1-yl]-hexyl}-benzamide;
3-Cyano-4-fluoro[F-19]-N-{6-[3-((2R,4S,5R)-4-hydroxy-5-hydroxymethyl-tetrahydro-thiophen-2-yl)5-methy-2,6,dioxo-3,6-dihydro-2H-pyrimidin-1-yl]-hexyl}-benzamide;
3-CN,4-F-Bz-Ava-Gln-Trp-Ala-Val-Gly-His-FA01010-Leu-NH2,
4F,3CN-Bnz-Arg-Ava-Gln-Trp-Ala-Val-NMeGly-His(3Me)-Sta-Leu-NH2,
3-CF3,4-F-Benzoyl-Arg-Ava-Gln-Trp-Ala-Val-NMeGly-His-Sta-Leu-NH2,
3-C N,4-F-Benzoyl-Arg-Ava-Gln-Trp-Ala-Val-Gly-His(3Me)-Sta-Leu-NH2,
3-CN,4-F-Benzoyl-Arg-Ava-Gln-Trp-Ala-Val-NMeGly-His-Sta-Leu-NH2, wherein F is ¹⁸F or ¹⁹F.

In a third aspect, the present invention refers to a method of preparing a compound having general chemical Formula II (method of fluorination) using an appropriate fluorination agent. The method comprises the (single) step of coupling a compound having general chemical Formula I with a fluorine isotope, more preferably with a radioactive or non-radioactive (“cold”) fluorine isotope derivative, even more preferably with ¹⁸F or ¹⁹F respectively and most preferably with ¹⁸F (radiofluorination). In the latter case the reagent to convert the compound having general chemical Formula I to the compound having general chemical Formula II is a fluorination agent. More preferably the compound having general chemical Formula II may thereafter be converted into a pharmaceutically acceptable salts of inorganic or organic acids thereof, hydrates, complexes, esters, amides, solvates and prodrugs thereof if desired. The reagents, solvents and conditions which can be used for this fluorination are common and well-known to the skilled person in the field. See, e.g., J. Fluorine Chem., 27 (1985):117-191.

In a preferred embodiment of the method, the compound having general chemical Formula I and its pharmaceutically acceptable salts of inorganic or organic acids thereof, hydrates, complexes, esters, amides, solvates and prodrugs thereof is any preferred compound described above for obtaining any preferred compound having general chemical Formula II, more specifically any preferred compound having general chemical Formulae IIA and IIB, or pharmaceutically acceptable salt, hydrate, ester, amide, solvate or prodrug thereof as described above.

In a preferred method of preparing a compound having general chemical Formula II, the step of fluorination more preferably radiofluorination of a compound having general chemical Formula I is carried out at a temperature at or below 90° C.

In a preferred method of preparing a compound of Formula II, the step of fluorination more preferably radiofluorination of a compound of Formula I is carried out at a temperature selected from a range from 10° C. to 90° C.

In a preferred embodiment, the method of fluorination more preferably radiofluorination occurs at a reaction temperature of from room temperature to 80° C.

In a preferred method of preparing a compound of Formula II, the step of fluorination more preferably radiofluorination of a compound of Formula I is carried out at a temperature selected from a range from 10° C. to 70° C.

In a preferred method of preparing a compound of Formula II, the step of fluorination more preferably radiofluorination of a compound of Formula I is carried out at a temperature selected from a range from 30° C. to 60° C.

In a preferred method of preparing a compound of Formula II, the step of fluorination more preferably radiofluorination of a compound of Formula I is carried out at a temperature selected from a range from 45 to 55° C.

In a preferred method of preparing a compound of Formula II, the step of fluorination more preferably radiofluorination of a compound of Formula I is carried out at a temperature at 50° C.

More preferably, the radioactive fluorine isotope derivate is 4,7,13,16,21,24-Hexaoxa-1,10-diazabicyclo[8.8.8]-hexacosane K18F (crownether salt Kryptofix K18F), K¹⁸F, H¹⁸F, KH¹⁸F₂, Cs¹⁸F, Na¹⁸ For tetraalkylammonium salt of ¹⁸F (e.g. [F-18]tetrabutylammonium fluoride). Most preferably, the a radioactive fluorine isotope derivate is K¹⁸F, H¹⁸F, or KH¹⁸F₂.

In a preferred embodiment, the fluorination agent is a non-radioactive fluorine isotope. More preferably, the non-radioactive fluorine isotope is ¹⁹F derivative, most preferably ¹⁹F.

In a preferred embodiment the solvents used in the present method may be DMF, DMSO, MeCN, DMA, DMAA, or mixture thereof, preferably the solvent is DMSO.

A new method is warranted in which the final product is prepared in a single step from the precursor. Only one purification step is necessary thereby the preparation can be accomplished in a short time (considering the half-life of ¹⁸F). In a typical prosthetic group preparation, very often temperatures of 100° C. and above are employed. The invention provides methods to accomplish the preparation at temperatures (80° C. or below) that preserve the biological properties of the final product. Additionally, single purification step is optionally carried out, thereby the preparation can be accomplished in a short time (considering the half-life of ¹⁸F).

In a tenth aspect the present invention refers to compounds having the general chemical Formula V:

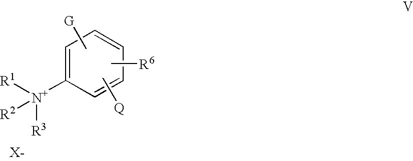

wherein N⁺(R¹)(R²)(R³), X⁻, -G, and -Q, have the same meaning as depicted above for compounds having general chemical Formula I. This includes in particular all preferred embodiments mentioned above with regard to the residues and substituents R¹, R², R³, X⁻, -G, and -Q, and all residues used to define these residues and substituents, such as R⁴, R⁵ and the like;

### R⁶ is C(O)OH.

In a preferred embodiment of compounds of Formula V, -G and -Q are independently from each other selected from —H, —CN, CF₃, and —Cl.

In a more preferred embodiment of compounds of Formula V, -G and -Q are independently from each other H, —CF₃, or CN.

In a even more preferred embodiment of compounds of Formula V, -G and -Q are independently from each other H, —CF₃, or —CN, whereas at least one member of the group comprising -G or -Q is —CF₃ or —CN.

Preferred compounds of Formula VI are selected from the group comprising

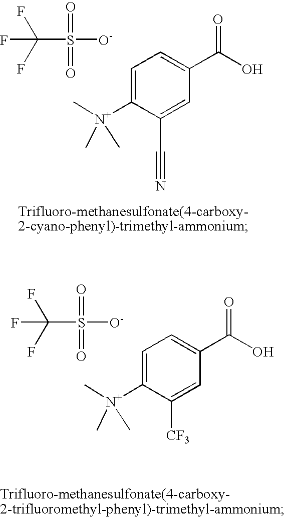

Compounds of Formula V are suited to be coupled to targeting agents towards compounds of Formula I which are starting materials for the radio labeling reaction towards compounds of Formula I or Formula A.

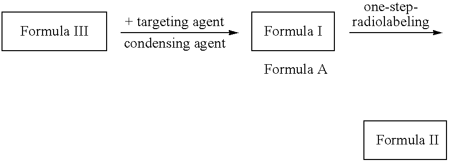

In a eleventh aspect the present invention refers to a method to synthesize compounds of Formula I (Formula A) wherein K is LG-O from compounds of Formula V. The method for obtaining a compound of formula I comprises the step of reacting a compound of formula V with a targeting agent, a condensing agent and a nucleophile wherein the targeting agent is selected from peptide, peptidomimetic, smaller organic molecule or oligonucleotide, condensing agent is selected from DCC, DIC, HBTU, HATU or TNTU and nucleophile is selected from HOBt, HOAt, HOSu, or N-hydroxy-5-norbornene-2,3-dicarboximid or LO-OH (LG is as defined above).

The condensing agent for the purpose of the present invention is a chemical substance capable of reacting with a carboxylic acid and an amine to result in the corresponding carboxylic amide, whereas the hydrate of the condensing agent is formed as a by-product. The term condensing agent refers to coupling agents, which are commonly used in peptide chemistry for the formation of peptide bonds and which are well known to a person skilled in the art (Fmoc Solid Phase Peptide Synthesis A practical approach, Edited by W. C. Chan and P. D. White, Oxford University Press 2000; Peptide Coupling Reagents: Names, Acronyms and References, Technical Reports, Vol. 4, No. 1, Albany Molecular Research, Inc., 1999).

Examples of condensing agents are DCC, DIC, HBTU, HATU, TNTU, and others mentioned in the above referenced publications.

The nucleophile for the purpose of the present invention is a group of atoms capable of forming a chemical bond with its reaction partner by donating both bonding electrons. More precisely, in this context the nucleophile is a N-hydroxy derivative or its anion, which is able to replace an aromatic trimethylammonium group during a typical peptide bond forming reaction (Fmoc Solid Phase Peptide Synthesis A practical approach, Edited by W. C. Chan and P. D. White, Oxford University Press 2000; Peptide Coupling Reagents: Names, Acronyms and References, Technical Reports, Vol. 4, No. 1, Albany Molecular Research, Inc., 1999). Representative examples for such nucleophiles are the in peptide synthesis commonly used activating additives HOBt, HOAt, HOSu, or N-hydroxy-5-norbornene-2,3-dicarboximid.

Compounds of Formula V can be condensed to targeting agents equipped with or without a spacer to obtain compounds of Formula I as defined above (Formula A) by using typical condensing agents which are known to persons skilled in the art. Suited condensing agents are for example DCC, DIC and 4-(4,6-dimethoxy-1,3,5-triazin-2-yl)-4-methylpiperidinium tetrafluoroborate (J. Am. Chem. Soc, 2005, 127, 48, 16912-16920). Examples for such a reaction are depicted in scheme 3 and 4.

### Example for Labeling:

¹⁸F-fluoride (up to 40 GBq) was azeotropically dried in the presence of Kryptofix 222 (5 mg in 1.5 ml MeCN) and cesium carbonate (2.3 mg in 0.5 ml water) by heating under a stream of nitrogen at 110-120° C. for 20-30 minutes. During this time 3×1 ml MeCN were added and evaporated. After drying, a solution of the precursor (2 mg) in 150 μl DMSO was added. The reaction vessel was sealed and heated at 50-70° C. for 5-15 mins to effect labeling. The reaction was cooled to room temperature and diluted with water (2.7 ml). The crude reaction mixture was analyzed using an analytical HPLC. The product was obtained by preparative radio HPLC to give to desired ¹⁸F labeled peptide.

In a fourth aspect, the present invention refers to a composition comprising a compound having general chemical Formula I or II, more specifically Formulae IIA and IIB, or a pharmaceutically acceptable salt, hydrate, ester, amide, solvate or prodrug thereof and further comprising a pharmaceutically acceptable carrier, diluent, excipient or adjuvant. Pharmaceutically acceptable carriers, diluents, excipients or adjuvants may include any and all solvents dispersion media, antibacterial and antifungal agents, isotonic agents, enzyme inhibitors, transfer ligands such as glucoheptonate, tartrate, citrate, or mannitol, and the like. Such compositions may be formulated as sterile, pyrogen-free, parenterally acceptable aqueous solution which may optionally be supplied in lyophilized form. The compositions of the invention may be provided as components of kits which may include buffers, additional vials, instructions for use, and the like.

In a fifth aspect, the present invention refers to a method of imaging diseases, wherein the method comprising introducing into a patient a detectable quantity of a labelled compound having general chemical Formula II, more specifically having general chemical Formula IIA, or a pharmaceutically acceptable salt, hydrate, ester, amide, solvate or prodrug thereof.

In a sixth aspect, the present invention refers to a kit comprising a sealed vial containing a predetermined quantity of a compound according to Formula I or a pharmaceutically acceptable salt, hydrate, ester, amide, solvate or prodrug thereof and optionally a pharmaceutically acceptable carrier, diluent, excipient or adjuvant. More preferably, the present invention relates to a kit comprising a compound or composition, as defined herein above, in powder form, and a container containing an appropriate solvent for preparing a solution of the compound or composition for the administration thereof to an animal, including a human.

In a seventh aspect, the present invention refers to a compound having general chemical Formula I or II, more specifically Formulae IIA and IIB, or a pharmaceutically acceptable salt, hydrate, ester, amide, solvate or prodrug thereof for use as medicament or as diagnostic imaging agent, more preferably for use as imaging agent for positron emission tomography (PET).

In an eighth aspect, the present invention refers to the use of a compound having general chemical Formula I or II, more specifically Formulae IIA and IIB, or a pharmaceutically acceptable salt, hydrate, ester, amide, solvate or prodrug thereof for the manufacture of a medicament or for the manufacture of a diagnostic imaging agent. In a more preferred embodiment the use concerns a medicament or a diagnostic imaging agent for treatment or positron emission tomography (PET) imaging, respectively. In an even more preferred embodiment, the use serves for imaging tissue at target site by the targeting agent.

The compounds of this invention are useful for the imaging of a variety of cancers including but not limited to carcinoma such as bladder, breast, colon, kidney, liver, lung, including small cell lung cancer, esophagus, gall-bladder, ovary, pancreas, stomach, cervix, thyroid, prostate and skin, hematopoetic tumors of lymphoid and myeloid lineage, tumors of mesenchymal origin, tumors of central peripheral nervous systems, other tumors, including melanoma, seminoma, teratocarcinoma, osteosarcoma, xeroderma pigmentosum, keratoxanthoma, thyroid follicular cancer and Karposi's sarcoma.

Most preferably, the use is for only for imaging of tumors, but also for imaging of inflammatory and/or neurodegenerative diseases, such as multiple sclerosis or Alzheimer's disease, or imaging of angiogenesis-associated diseases, such as growth of solid tumors, and rheumatoid arthritis.

More specifically, as far as the compound having general chemical Formula A comprises bombesin or bombesin analogs, this compound binds specifically to human GRP receptors present in prostate tumor, breast tumor and metastasis.

Further, the compounds having general chemical Formula II, in which W is ¹⁹F or other non-radioactive (“cold”) halogen elements may be used in biological assays and chromatographic identification. More preferably, the invention relates to the use of a compound having general chemical Formula I for the manufacture of a compound having general chemical Formula IIB as a measurement agent.

The compounds having general chemical Formulae I and II and the respective pharmaceutically acceptable salts, hydrates, esters, amides, solvates or prodrugs thereof of the invention can be chemically synthesized in vitro. In case P is selected to be a peptide, such peptides can generally advantageously be prepared on a peptide synthesizer. Preferably, particularly when B-D is a sequence of amino acids and P is a peptide and both together are forming a fusion peptide, said fusion peptide may be synthesized sequentially, i.e., the part comprising the amino acid sequence 3-D and the targeting agent P may be obtained by subsequently adding suitable activated and protected amino acid derivatives or preformulated amino acid sequences to the growing amino acid chain. For details regarding peptide synthesis it can be referred to, e.g., B. Gutte “Peptides: Synthesis, Structures, and Applications”, Academic Press, 1995; X. C. Chan et al. “Fmoc Solid Phase Peptide Synthesis; A Practical Approach”, Oxford University Press, 2000; J. Jones “Amino Acid and Peptide Synthesis”, 2^{nd} ed., Oxford University Press, 2002; M. Bodanszky et al., “Principles of Peptide Synthesis”, 2^{nd} ed., Springer, 1993.

The radioactively labelled compounds having general chemical Formula II provided by the invention may be administered intravenously with any pharmaceutically acceptable carrier, e.g., with conventional medium such as an aqueous saline medium, or in blood plasma medium, as a pharmaceutical composition for intravenous injection. Such medium may also contain conventional pharmaceutical materials such as, for example, pharmaceutically acceptable salts to adjust the osmotic pressure, buffers, preservatives and the like. Among the preferred media are normal saline and plasma. Suitable pharmaceutical acceptable carriers are known to the person skilled in the art. In this regard reference can be made to, e.g., Remington's Practice of Pharmacy, 11^{th} ed. and in J. of. Pharmaceutical Science & Technology, Vol. 52, No. 5, September-Oct., p. 238-311 see table page 240 to 311, both publication include herein by reference.

The concentration of the compound having general chemical Formula II and the pharmaceutically acceptable carrier, for example, in an aqueous medium, varies with the particular field of use. A sufficient amount is present in the pharmaceutically acceptable carrier when satisfactory visualization of the imaging target (e.g., a tumor) is achievable.

In accordance with the present invention, the radiolabelled compounds having general chemical Formula II either as a neutral complex or as a salt with a pharmaceutically acceptable counterion are administered in a single unit injectable dose. Any of the common carriers known to those with skill in the art, such as sterile saline solution or plasma, can be utilized after radiolabeling for preparing the injectable solution to diagnostically image various organs, tumors and the like in accordance with the invention. Generally, the unit dose to be administered for a diagnostic agent has a radioactivity of about 0.1 mCi to about 100 mCi, preferably 1 mCi to 20 mCi. For a radiotherapeutic agent, the radioactivity of the therapeutic unit dose is about 10 mCi to 700 mCi, preferably 50 mCi to 400 mCi. The solution to be injected at unit dosage is from about 0.01 ml to about 30 ml. For diagnostic purposes after intravenous administration, imaging of the organ or tumor in vivo can take place in a matter of a few minutes. Preferably, imaging takes place between two minutes and two hours, after injecting into patients. In most instances, a sufficient amount of the administered dose will accumulate in the area to be imaged within about 0.1 of an hour to permit the taking of scintigraphic images. Any conventional method of scintigraphic imaging for diagnostic purposes can be utilized in accordance with this invention.

In general, compounds having general chemical Formula II can be generated from compounds having general chemical Formula I by labeling compounds having general chemical Formula I with fluorine isotope, more preferably with ¹⁸F, or ¹⁹F, and most preferably with ¹⁸F. Methods and conditions for such labeling reactions are well known to the skilled person (F. Wüst, C. Hultsch, R. Bergmann, B. Johannsen and T. Henle. Appl. Radiat. Isot., 59, 43-48 (2003); Y. S. Ding, C. Y. Shiue, J. S. Fowler, A. P. Wolf and A. J. Plenevaux, Fluorine Chem., 48, 189-205 (1990).

Scheme 3 illustrates a generally applicable synthetic route for generating a compound having general chemical Formula I and subsequent radiolabeling of this compound with for example ¹⁸F or ¹⁹F in order to arrive at a compound having general chemical Formula II. Scheme 3 depicts the formation of an O-benzotriazolyl substituted aromatic moiety connected to a peptide, compound 1, which is to be understood as a general representative of any compound having general chemical Formula I, and subsequent direct radiolabeling towards the corresponding ¹⁸F- or ¹⁹F-labelled compound 2, respectively, which represents a compound having general chemical Formula II. Compound 1, containing an O-benzotriazolyl moiety is prepared by 1-hydroxybenzo-triazole mediated coupling of trimethylammonium benzoic acid, compound 3, to a resin bound protected peptide with the concomitant displacement of trimethylammonium with O-benzotriazole. Compound 1 was obtained by the cleavage from the resin according to well known methods in peptide chemistry (W. C. Chan and P. D. White (Editors) “Fmoc Solid Phase Peptide Synthesis”, Oxford University Press (2000), and references therein). The oxabenzotrizole moiety can be displaced by ¹⁸F or ¹⁹F under standard conditions (F. Wüst, C. Hultsch, R. Bergmann, B. Johannsen and T. Henle. Appl. Radiat. Isot., 59, 43-48 (2003); Y. S. Ding, C. Y. Shiue, J. S. Fowler, A. P. Wolf and A. J. Plenevaux, Fluorine Chem., 48, 189-205 (1990). The oxabenzotrizole moiety can also be substituted with cold fluoride (¹⁹F). In general, this method is applicable to the generation of all compounds having general chemical Formula I and to the subsequent radiolabeling of such compounds in order to arrive at all compounds having general chemical Formula II.

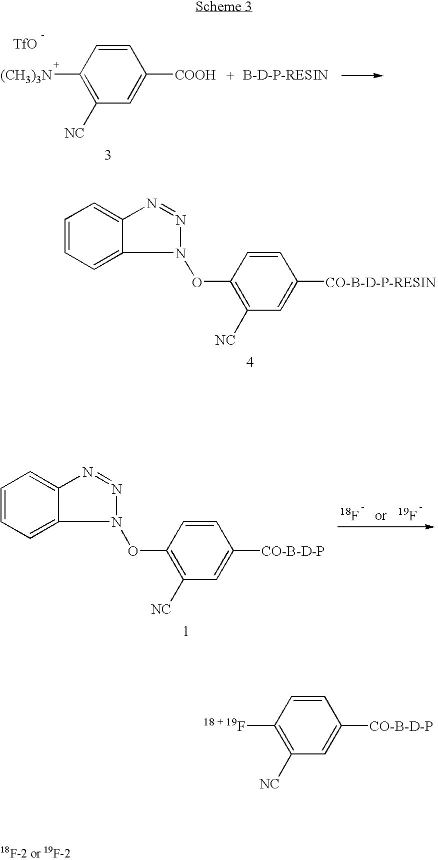

Scheme 4 depicts an alternative method for generating a compound having general chemical Formula I. According to this method, 4-oxobenzotriazolylbenzoic acid, compound 6, can be prepared independently, and is coupled later to the terminus of resin bound B-D-P. Compound 1, which is to be understood as a general representative of any compound having general chemical Formula I, was obtained by the cleavage from the resin according to well known methods in peptide chemistry. In general this method is applicable to the generation of all compounds having general chemical Formula I.

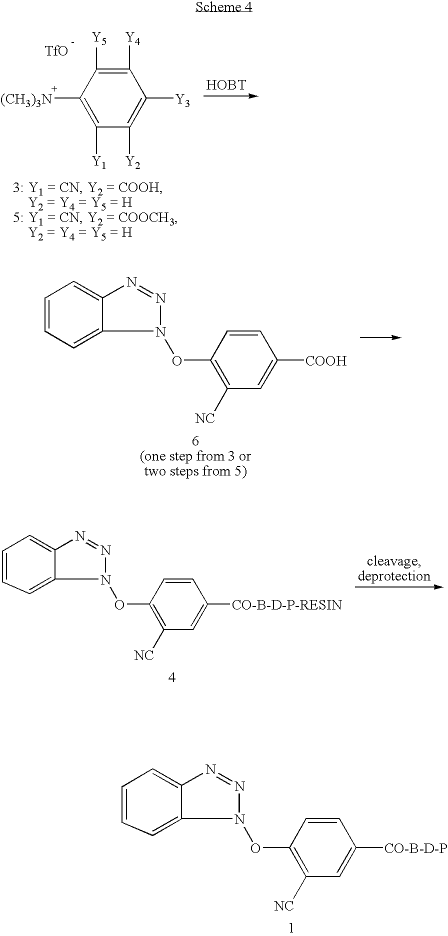

The invention also refers to two other independent methods for the preparation of compounds having general chemical Formula I. These methods are illustrated in Schemes 5 and 6. Again, these methods are applicable to the generation of all compounds having general chemical Formula I.

The intermediate 6 can also be prepared from the corresponding boronic acids 7 by copper promoted displacement, according to, e.g., the general method described in P. Y. S. Lam, G. Charles, C. G. Clark, S. Saubern, J. Adams, M. Kristin, K. M. Averill, M. T. Chan, A. Combs. “Copper Promoted Aryl/Saturated Heterocyclic C—N Bond Cross-Coupling with Arylboronic Acid and Arylstannane” SynLett., 5, 674 (2000).

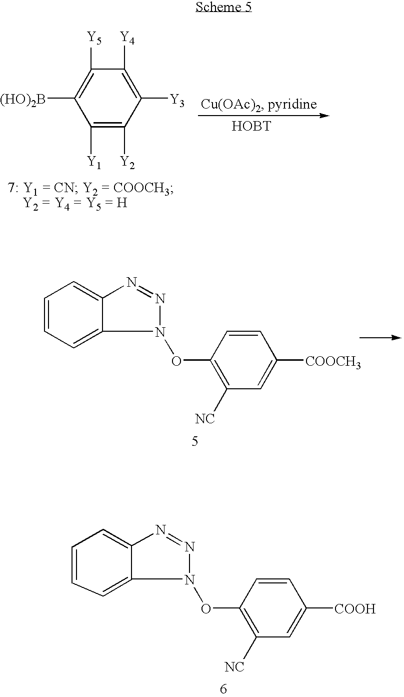

Compound 6 is converted to compound 1, which is to be understood as a general representative of any compound having general chemical Formula I, as shown in scheme 4.

Compound 1, which is to be understood as a general representative of any compound having general chemical Formula I, can also be prepared in solid phase as shown in Scheme 6.

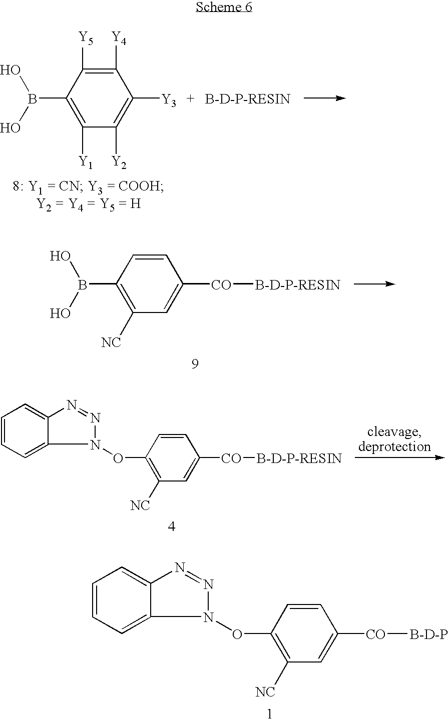

Without further elaboration, it is believed that one skilled in the art can, using the preceding description, utilize the present invention to its fullest extent. The following preferred specific embodiments are therefore to be construed as merely illustrative, and not limitative of the remainder of the disclosure in any way whatsoever. The following Examples can be repeated with similar success by substituting the generically or specifically described reactants and/or operating conditions of this invention for those used in the preceding examples.

### EXAMPLES

The compounds having general chemical Formula I of the present invention can be synthesized depending on the nature of the moiety LG-O—(C₆Y¹Y²Y³Y⁴)-(??). The peptide portion of -A-B-D-P can conveniently be prepared according to generally established techniques known in the art of peptide synthesis, such as solid-phase peptide synthesis. They are amenable Fmoc-solid phase peptide synthesis, employing alternate protection and deprotection. These methods are well documented in peptide literature. (Reference; “Fmoc Solid Phase Peptide Synthesis A practical approach”, Edited by W. C. Chan and P. D. White, Oxford University Press 2000) (For Abbreviations see Descriptions).

### General

Peptide synthesis was carried out using Rink-Amide resin (0.68 mmol/g) following standard Fmoc strategy (G. B. Fields, R. L. Noble, “Solid phase peptide synthesis utilizing 9-fluorenylmethoxycarbonyl amino acids”, Int. J. Pept. Protein Res., 1990; 35: 161-214). All amino acid residues were, if not further specified, L-amino acid residues.

### Fmoc-Deprotection (General Procedure)

The resin-bound Fmoc peptide was treated with 20% piperidine in DMF (v/v) for 5 min and a second time for 20 min. The resin was washed with DMF (2×), CH₂Cl₂ (2×), and DMF (2×).

### HBTU/HOBT Coupling (General Procedure)

A solution of Fmoc-Xaa-OH (4 eq), HBTU (4 eq), HOBT (4 eq), DIEA (4 eq) in DMF was added to the resin-bound free amine peptide and shaken for 90 min at room temperature. The coupling was repeated for another 60 min and the resin was washed with DMF (2×), CH₂Cl₂ (2×), and DMF (2×).

### Radiolabeling (General Procedure)

No-carrier-added aqueous [¹⁸F]fluoride ion was produced by irradiation of [¹⁸O]H₂O via the ¹⁸O (p, n)¹⁸F nuclear reaction. Resolubilization of the aqueous [¹⁸F]fluoride (500-1500 MBq) was accomplished by filtration through a QMA SepPak which was preconditioned with 5 ml 0.5M K₂CO₃, washed with 5 ml water, and dried by pushing through air. 100 μl of the ¹⁸F were passed through the SepPak and dried by pushing through air. The ¹⁸F was eluted into a conical vial with 4 ml Kryptofix 2.2.2®/MeCN/K₂CO₃/water mixture. The resulting solution (50-500 MBq) was dried azeotropically four times in an N₂ stream at 120° C. To the vial containing anhydrous [¹⁸F]fluoride was added the fluorination precursor (1-4 mg) in DMSO (300-500 μl). After incubation at 50-70° C. for 15-60 min, the crude reaction mixture was analyzed using an analytical HPLC (Column Zorbax SB C18, 50×4.6 mm, 1.8μ, 2 ml/min, solvent A: H₂O, solvent B: MeCN, gradient: 5%-95% B in 7 min or Column Econosphere C18, 53×7 mm, 3μ, 3 ml/min (Alltech), solvent A: H₂O+0.1% TFA, solvent B: MeCN/H₂O 9/1+0.1% TFA, gradient: 5-95% B in 7 min).

### Synthesis and Labeling of 4-(Benzotriazol-1-yloxy)-3-cyano-benzoyl-valyl-β-alanyl-phenylalanyl-glycine amide (1a, Example 1, cf. scheme 3)

4-(Benzotriazol-1-yloxy)-3-cyano-benzoyl-valyl-β-alanyl-phenylalanyl-glycine amide was synthesized from the corresponding resin bound tetrapeptide and (4-carboxy-2-cyano-phenyl)-trimethyl-ammonium trifluoromethanesulfonate followed by cleavage and deprotection as shown below.

The peptide was fluorinated with [¹⁸F]potassium fluoride in the presence of K₂CO₃ and Kryptofix 2.2.2® in DMSO to yield ¹⁸F-labelled peptide.

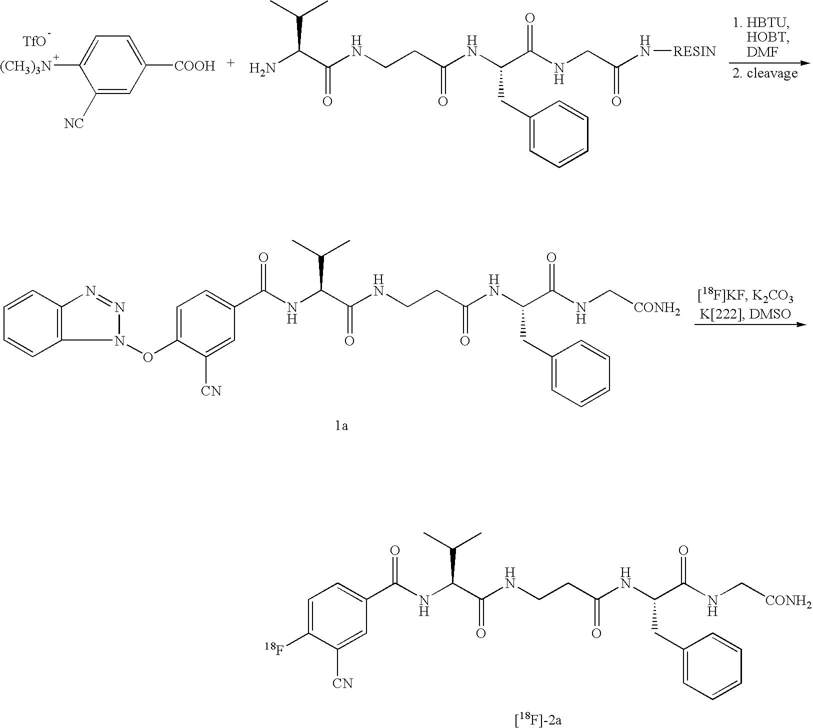

The resin-bound tetrapeptide was prepared according to the above described general procedures. The solution of (4-carboxy-2-cyano-phenyl)-trimethyl-ammonium trifluoromethanesulfonate (4 eq), HBTU (4 eq), HOBT (4 eq) and DIPEA (4 eq) in DMF was added to the resin-bound free amine tetrapeptide and shaken for 4 h at ambient temperature. The resin was washed with DMF (4×) and CH₂Cl₂ (4×) and dried in vacuum. The peptide was cleaved from resin by treatment with a mixture of TFA, water, phenol and triisopropylsilane (85:5:5:5 v-%). The peptide was then precipitated with methyl-tert-butyl ether, the solvent was removed by centrifugation, and the crude product was purified by RP-HPLC. The purified product (1a) was analyzed by RP-HPLC (5-95% acetonitrile/12 min): tᵣ=6.72 min, and ESI-MS: m/z=654.2 (M+H)⁺.

Labeling was performed according to the above described general procedure. The F-18 labeled product ([¹⁸F]-2a) was confirmed by co-injection with the non-radioactive F-19 fluoro standard [¹⁹F]-2a on the Econsphere analytical HPLC.

### Synthesis of 3-cyano-4-fluoro-benzoyl-valyl-D-alanyl-phenylalanyl-glycine amide (F-19 fluoro standard [¹⁹F]-2a)

The resin-bound tetrapeptide (H-valyl-β-alanyl-phenylalanyl-glycinyl-Rink amide resin) was prepared according to the above described general procedures. The solution of 3-Cyano-4-fluoro-benzoic acid (4 eq), HBTU (4 eq), HOBT (4 eq) and DIPEA (4 eq) in DMF was added to the resin-bound free amine tetrapeptide and shaken for 4 h at ambient temperature. The resin was washed with DMF (4×) and CH₂Cl₂ (4×) and dried in vacuum. The peptide was cleaved from resin by treatment with a mixture of TFA, water, phenol and triisopropylsilane (85:5:5:5 v-%). The peptide was then precipitated with methyl-tert-butyl ether, the solvent was removed by centrifugation, and the crude product was purified by RP-HPLC. The purified product ([¹⁹F]-2a) was analyzed by RP-HPLC (5-95% acetonitrile/12 min): tᵣ=6.03 min, and ESI-MS: m/z=539.1 (M+H)⁺.

### Synthesis and Labeling of 4-(Benzotriazol-1-yloxy)-3-cyano-benzoyl-valyl-β-alanyl-histidyl(π-Me)-glycine amide (1b, Example 2, cf. scheme 3)

4-(Benzotriazol-1-yloxy)-3-cyano-benzoyl-valyl-β-alanyl-histidyl(π-Me)-glycine amide was synthesized from the corresponding resin bound tetrapeptide and (4-carboxy-2-cyano-phenyl)-trimethyl-ammonium trifluoromethanesulfonate followed by cleavage and deprotection as shown below.

The peptide was fluorinated with [¹⁸F]potassium fluoride in the presence of K₂CO₃ and Kryptofix 2.2.2® in DMSO to yield ¹⁸F-labelled peptide.

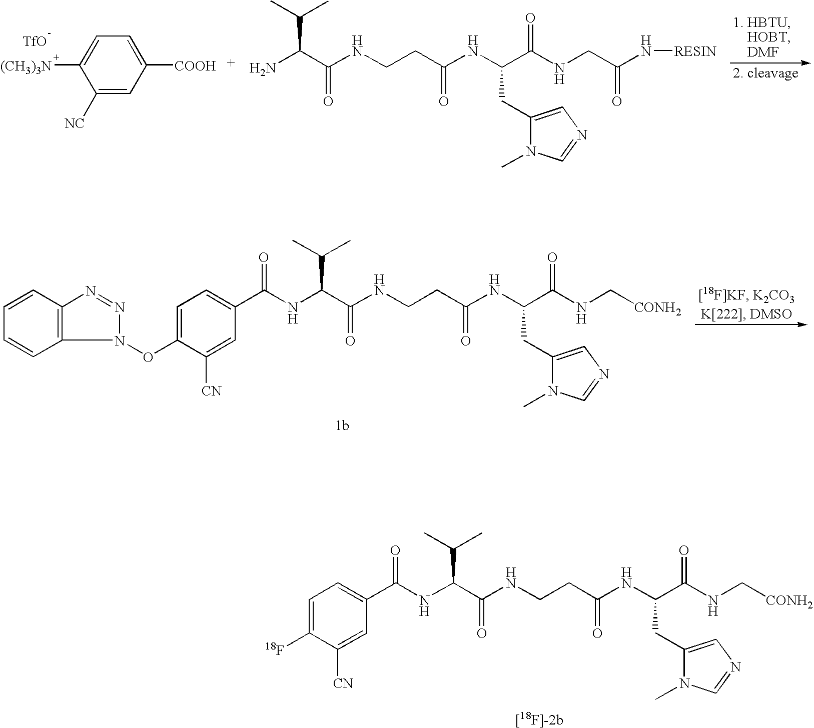

The resin-bound tetrapeptide was prepared according to the above described general procedures. The solution of (4-carboxy-2-cyano-phenyl)-trimethyl-ammonium trifluoromethanesulfonate (4 eq), HBTU (4 eq), HOBT (4 eq) and DIPEA (4 eq) in DMF was added to the resin-bound free amine tetrapeptide and shaken for 12 h at ambient temperature. The resin was washed with DMF (4×) and CH₂Cl₂ (4×) and dried in vacuum. The peptide was cleaved from resin by treatment with a mixture of TFA, water, phenol and triisopropylsilane (85:5:5:5 v-%). The peptide was then precipitated with methyl-tert-butyl ether, the solvent was removed by centrifugation, and the crude product was purified by RP-HPLC The purified product (1b) was analyzed by RP-HPLC (5-95% acetonitrile/12 min): tᵣ=5.22 min, and ESI-MS: m/z=658.1 (M+H)⁺.

Labeling was performed according to the above described general procedure. The F-18 labeled product ([¹⁸F]-2b) was confirmed by co-injection with the non-radioactive F-19 fluoro standard ([¹⁹F]-2b) on the Econsphere analytical HPLC.

### Synthesis of 3-cyano-4-fluoro-benzoyl-valyl-α-alanyl-histidyl(π-Me)-glycine amide (F-19 fluoro standard [¹⁹F]-2b)

The resin-bound tetrapeptide (H-valyl-1-alanyl-histidyl(π-Me)-glycinyl-Rink amide resin) was prepared according to the above described general procedures. The solution of 3-cyano-4-fluoro-benzoic acid (4 eq), HBTU (4 eq), HOBT (4 eq) and DIPEA (4 eq) in DMF was added to the resin-bound free amine tetrapeptide and shaken for 4 h at ambient temperature. The resin was washed with DMF (4×) and CH₂Cl₂ (4×) and dried in vacuum. The peptide was cleaved from resin by treatment with a mixture of TFA, water, phenol and triisopropylsilane (35:5:5:5 v-%). The peptide was then precipitated with methyl-tert-butyl ether, the solvent was removed by centrifugation, and the crude product was purified by RP-HPLC. The purified product ([¹⁹F]-2b) was analyzed by RP-HPLC (5-95% acetonitrile/12 min); tᵣ=4.45 min, and ESI-MS: m/z=543.1 (M+H)⁺.

### Synthesis and Labeling of 3-cyano-4-([1,2,3]triazolo[4,5-b]pyridin-3-yloxy)-benzoyl-(5-amide (10, Example 3, cf. scheme 3)

3-Cyano-4-([1,2,3]triazolo[4,5-b]pyridin-3-yloxy)-benzoyl-(5-aminopentanoyl)-phenylalanyl-(4(S)-amino-3(S)-hydroxy-6-methyl)heptanoyl-leucine amide was synthesized from the corresponding resin bound tetrapeptide and (4-carboxy-2-cyano-phenyl)-trimethyl-ammonium trifluoromethanesulfonate followed by cleavage and deprotection as shown below. The peptide was fluorinated with [¹⁸F]potassium fluoride in the presence of K₂CO₃ and Kryptofix 2.2.2® in DMSO to yield ¹⁸F-labelled peptide.

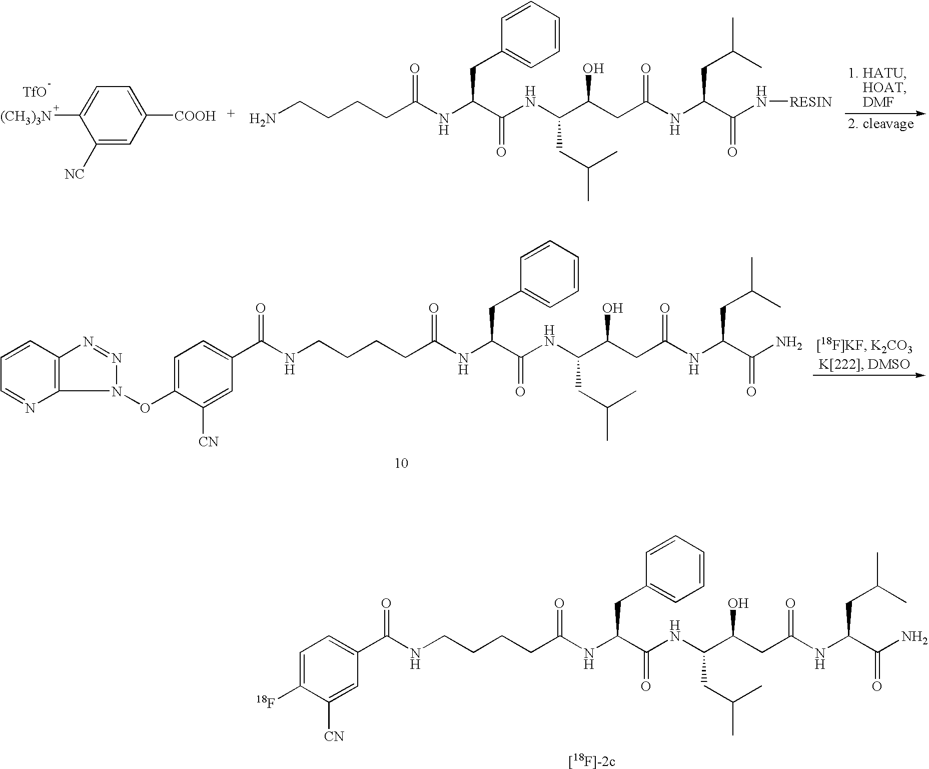

The resin-bound tetrapeptide was prepared according to the above described general procedures. The solution of (4-carboxy-2-cyano-phenyl)-trimethyl-ammonium trifluoromethanesulfonate (4 eq), HATU (4 eq), HOAT (4 eq) and DIPEA (4 eq) in DMF was added to the resin-bound free amine tetrapeptide and shaken for 12 h at ambient temperature. The resin was washed with DMF (4×) and CH₂Cl₂ (4×) and dried in vacuum. The peptide was cleaved from resin by treatment with a mixture of TFA, water, phenol and triisopropylsilane (85.5:5:5 v-%). The peptide was then precipitated with methyl-tert-butyl ether, the solvent was removed by centrifugation, and the crude product was purified by RP-HPLC. The purified product (10) was analyzed by RP-HPLC (5-95% acetonitrile/12 min): tᵣ=6.33 min, and ESI-MS: m/z=797.4 (M+H)⁺.

Labeling was performed according to the above described general procedure. The F-18 labeled product ([¹⁸F]-2c) was confirmed by co-injection with the non-radioactive F-19 fluoro standard ([¹⁹F]-2c) on the Econsphere analytical HPLC.

### Synthesis of 3-cyano-4-fluoro-benzoyl-(5-aminopentanoyl)-phenylalanyl-(4(S)-amino-3(S-hydroxy-6-methyl)heptanoyl-leucine amide (F-19 fluoro standard [¹⁹F]-2c)

The resin-bound tetrapeptide (H-(5-aminopentanoyl)-phenylalanyl-(4(S)-amino-3(S)-hydroxy-6-methyl)heptanoyl-leucinyl-Rink amide resin) was prepared according to the above described general procedures. The solution of 3-cyano-4-fluoro-benzoic acid (4 eq), HBTU (4 eq), HOBT (4 eq) and DIPEA (4 eq) in DMF was added to the resin-bound free amine tetrapeptide and shaken for 4 h at ambient temperature. The resin was washed with DMF (4×) and CH₂Cl₂ (4×) and dried in vacuum. The peptide was cleaved from resin by treatment with a mixture of TFA, water, phenol and triisopropylsilane (85:5:5:5 v-%). The peptide was then precipitated with methyl-tert-butyl ether, the solvent was removed by centrifugation, and the crude product was purified by RP-HPLC. The purified product ([¹⁹F]-2c) was analyzed by RP-HPLC (5-95% acetonitrile/12 mm): tᵣ=6.35 min, and ESI-MS: m/z=681.1 (M+H)⁺.

### Synthesis of 4-(benzotriazol-1-yloxy)-3-cyano-benzoic acid methyl ester (11, Example 4, cf. scheme 4)

4-(Benzotriazol-1-yloxy)-3-cyano-benzoic acid methyl ester was synthesized from the (2-cyano-4-methoxycarbonyl-phenyl)-trimethyl-ammonium trifluoromethanesulfonate as shown below.

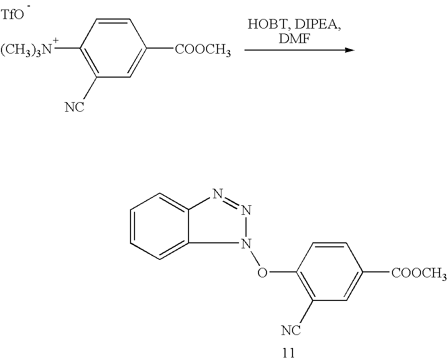

3-Cyano-4-(trimethylammonium)benzoic acid methylester trifluoromethanesulfonate, HOBT and DIPEA and were dissolved in DMF and stirred for 8 h. The solvent was removed and the residue was purified by RP-HPLC. The purified product (11) was analyzed by RP-HPLC (5-95% acetonitrile/12 min): tᵣ=8.62 min, and ESI-MS: m/z=295.0 (M+H)⁺.

### Synthesis of 4-(benzotriazol-1-yloxy)-3-chloro-benzoyl-valyl-β-alanyl-phenylalanyl-glycine amide (12, example 5, cf. scheme 6),

4-(Benzotriazol-1-yloxy)-3-chloro-benzoyl-valyl-β-alanyl-phenylalanyl-glycine amide was synthesized from the corresponding resin bound tetrapeptide and 2-chloro-4-carboxy-phenylboronic acid followed by copper-mediated displacement of the boronic acid moiety with HOBT and subsequent cleavage as shown below.

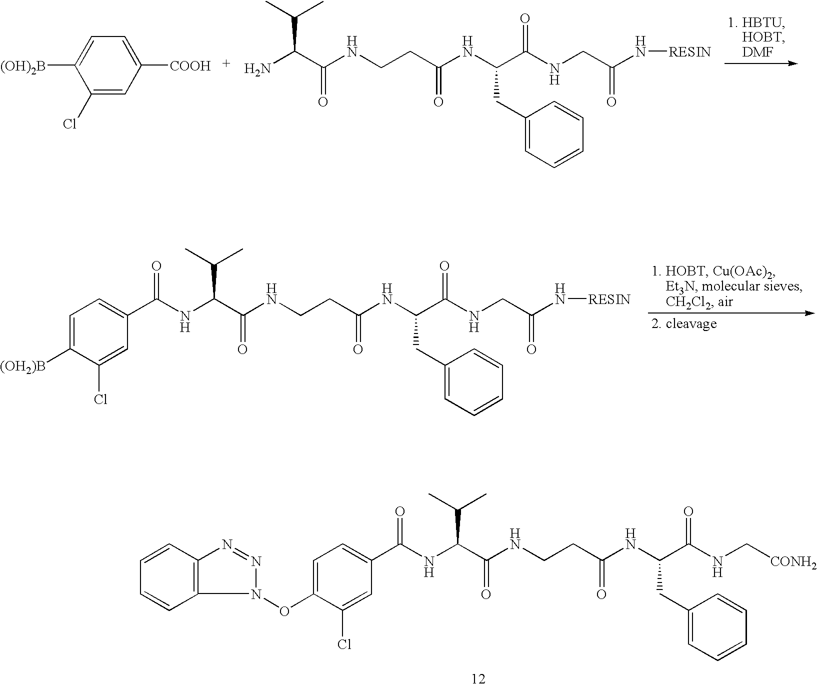

The resin-bound tetrapeptide was prepared according to the above described general procedures. The boronic acid derivative (4 eq) was solved in DMF together with HBTU (4 eq), HOBT (4 eq) and DIPEA (4 eq). The solution was shaken with the resin-bound tetrapeptide for 4 h. The resin was then washed with DMF (4×) and CH₂Cl₂ (4×). The resin was then shaken with solution of HOBT (4 eq), copper(II) acetate (6 eq) and triethylamine (8 eq) in CH₂Cl₂, and 4 Å molecular sieves for 48 h at ambient temperature. During the reaction the solution was exposed to air. The resin was then washed with DMF (4×) and CH₂Cl₂ (4×) and dried in vacuo. Cleavage of the product from resin was achieved by treatment with TFA/water (80: 20 v-%) for 2 h. The product was precipitated with methyl-tert-butyl ether, the solvent was removed by centrifugation, and the crude product was purified by RP-HPLC. The purified product (12) was analyzed by RP-HPLC (5-95% acetonitrile/12 min): tᵣ=5.79 min and ESI-MS: m/z=663.2 (M+H)⁺.

### Synthesis of 5-[3-cyano-4-(2,5-dioxo-pyrrolidin-1-yloxy)-benzoylamino]-(5 aminopentanoyl)-octapeptide amide (13, Example 6, cf. scheme 3)

5-[3-Cyano-4-(2,5-dioxo-pyrrolidin-1-yloxy)-benzoylamino]-(5-aminopentanoyl)-octa-peptide amide was synthesized from the corresponding resin bound nonapeptide and (4 carboxy-2-cyano-phenyl)-trimethyl-ammonium trifluoromethanesulfonate followed by cleavage and deprotection as shown below. The peptide was fluorinated with [¹⁹F]potassium fluoride in the presence of K₂CO₃ and Kryptofix 2.2.2® in DMSO to yield ¹⁹F-labelled peptide.

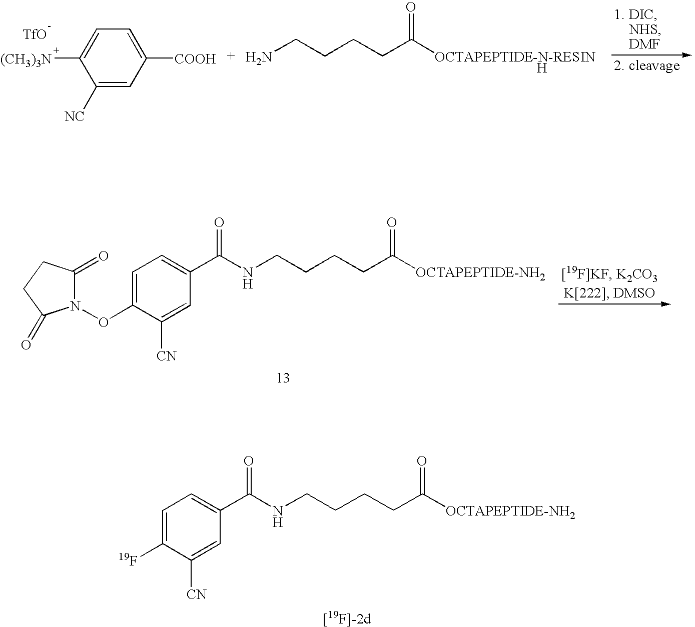

The resin-bound nonapeptide was prepared according to the above described general procedures. The solution of (4-carboxy-2-cyano-phenyl)-trimethyl-ammonium trifluoro-methanesulfonate (4 eq), diisopropylcarbodiimide (DIC, 4 eq), N-hydroxysuccinimide (NHS, 4 eq) and DIPEA (4 eq) in DMF was added to the resin-bound free amine nonapeptide and shaken for 12 h at ambient temperature. The resin was washed with DMF (4×) and CH₂Cl₂ (4×) and dried in vacuum. The peptide was cleaved from resin by treatment with a mixture of TFA, water, phenol and triisopropylsilane (85:5:5:5 v-%). The peptide was then precipitated with methyl-tert-butyl ether, the solvent was removed by centrifugation, and the crude product was purified by RP-HPLC. The purified product (13) was confirmed by RP-HPLC and ESI-MS. Compound 13 may be fluorinated with [¹⁹F]potassium fluoride according to the above described method. Fluorinated product [¹⁹F]-2d could be confirmed by HPLC-MS of the crude reaction mixture.

For the following procedures, LG was selected from the group comprising

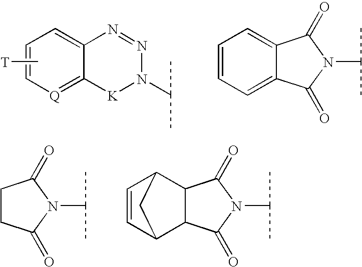

wherein T is H or Cl, Q is CH or N, K is absent or C═O, having general chemical Formula I.

### Procedure for the Displacement of the Trimethylamino Group by a N-hydroxy-type Leaving Group (LGOH)

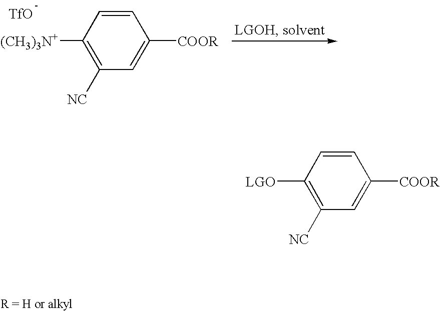

3-Cyano-4-(trimethylammonio)benzoic acid or a corresponding alkyl ester thereof was solved in DMF, DMSO, acetonitrile, DMPU or any solvent suitable for a nucleophilic aromatic substitution reaction. To this solution was added the N-hydroxy-type leaving group according to the above definition. A base like tertiary amine (triethylamine, DIPEA), potassium carbonate, or sodium hydride or a comparable base may be added. The solution was then stirred at ambient temperature, elevated temperature or under microwave conditions. The product was obtained after removal of the solvent and purification of the crude by reversed phase or normal phase chromatography.

### Procedure for the Displacement of a Boronic Acid Group by a N-Hydroxy-Type Leaving Group (LGOH)

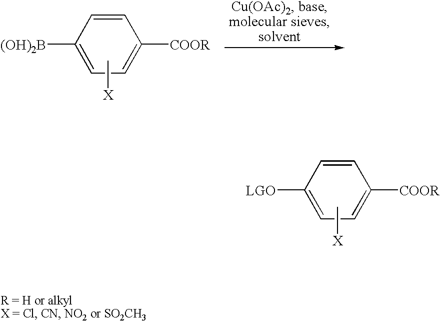

The substituted 4-carboxyphenylboronic acid or a corresponding alkylcarboxylic ester thereof was solved in either CH₂Cl₂, DMF, DMSO, acetonitrile, DMPU or mixtures thereof. To this solution was added the N-hydroxy-type leaving group according to the above definition, an amine base like triethylamine, DIPEA or pyridine, copper(II) acetate or a comparable copper salt, and molecular sieves. Ionic liquid (BMI or related) could be added. The solution was then stirred at ambient temperature in the presence of air or molecular oxygen. Alternatively the reaction can be carried out using an oxidative agent like TEMPO, possibly under elevated temperature. The product was obtained after removal of the solvent and purification of the crude by reversed phase or normal phase chromatography.

Procedure for the Saponification of 3-cyano-4-(LGO)-benzoic Acid Alkyl Esters:

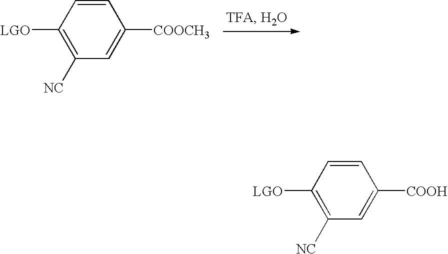

The alkyl ester was treated with a mixture of TFA and water under ambient or elevated temperature. Subsequently, the solvent was removed and the crude benzoic acid was purified by normal phase or reversed phase chromatography. The benzoic acid derivative was coupled to a resin-bound free amine peptide using one of various standard coupling conditions known in the literature.

### Analytical Data for Non-Radioactive Compounds

Compounds were analyzed on a Purosher® C-18, 4×125 mm, 5 μm pore size, 1 ml/min, solvent A: H₂O+0.1% TFA, solvent B: MeCN+0.1% TFA, gradient: 5-95% B in 12 min. Products were confirmed by ESI-MS. Purity was assessed by UV (215 nm). The following Table summarizes retention times and observed ESI-MS signals of the shown compounds.

| | Retention | |
|---|---|---|
| Preparative Example | Time | [M + H]⁺ |
| 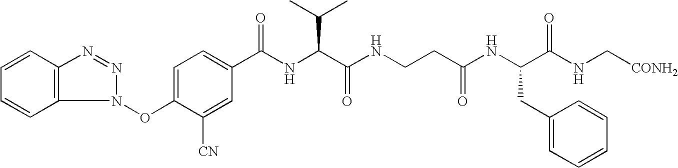 | 6.72 min | 654.2 |
| 1a | | |
| 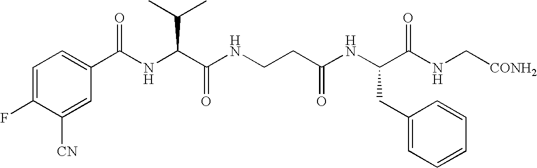 | 6.03 min | 539.1 |
| [¹⁹F]-2a | | |
| 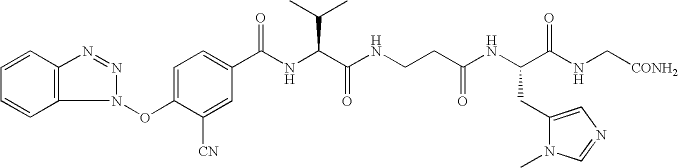 | 5.22 min | 658.1 |
| 1b | | |
| 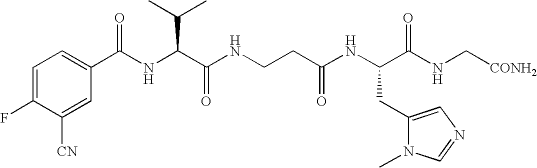 | 4.45 min | 543.1 |
| [¹⁹F]-2b | | |
| 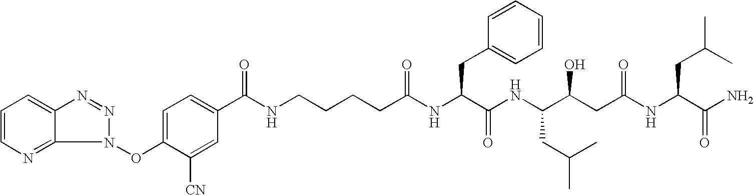 | 6.33 min | 797.4 |
| 10 | | |
| 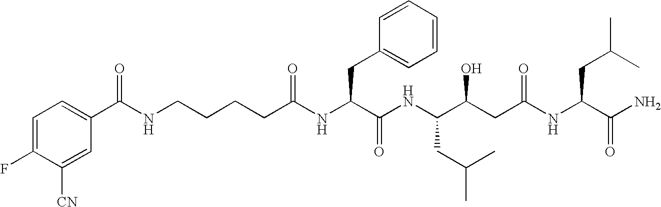 | 6.35 min | 681.1 |
| [¹⁹F]-2c | | |
| 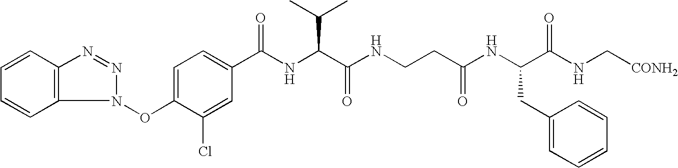 | 5.79 min | 663.2 |
| 12 | | |

Analysis of F-18-Fluorinated Compounds and Comparison with Labelling of the Corresponding Trimethylammonium Precursor

The identity of F-18 radiolabelled products was confirmed by coinjection with the non-radioactive F-19 fluoro standard on the Econospher analytical HPLC (see general procedure for radiolabeling).

FIG. 1 shows the radiotrace of the crude reaction mixture after incubating precursor 1a and “F-18” according to the above described general procedure for radiolabeling for 60 min.

FIG. 2 shows the radiotrace of the crude reaction mixture after incubating precursor 13 and “F-18” according to the above described general procedure for radiolabeling for 60 min for comparison.

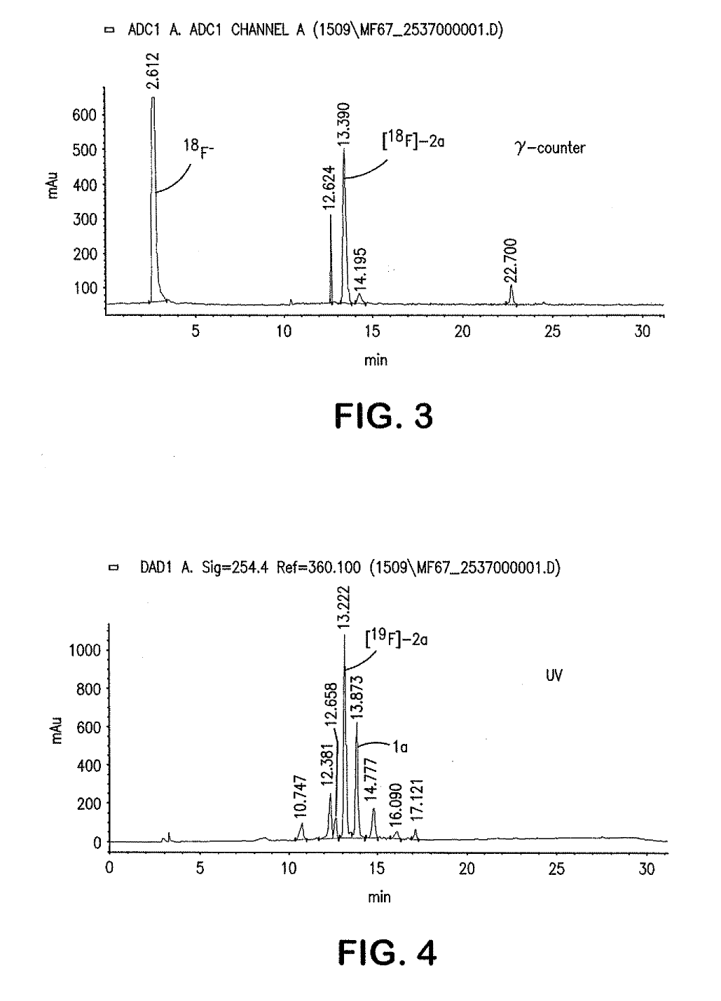

FIG. 3 shows radio- and UV-trace of the reaction according to FIG. 1 coinjected with the F-19 fluoro standard [¹⁹F]-2a.

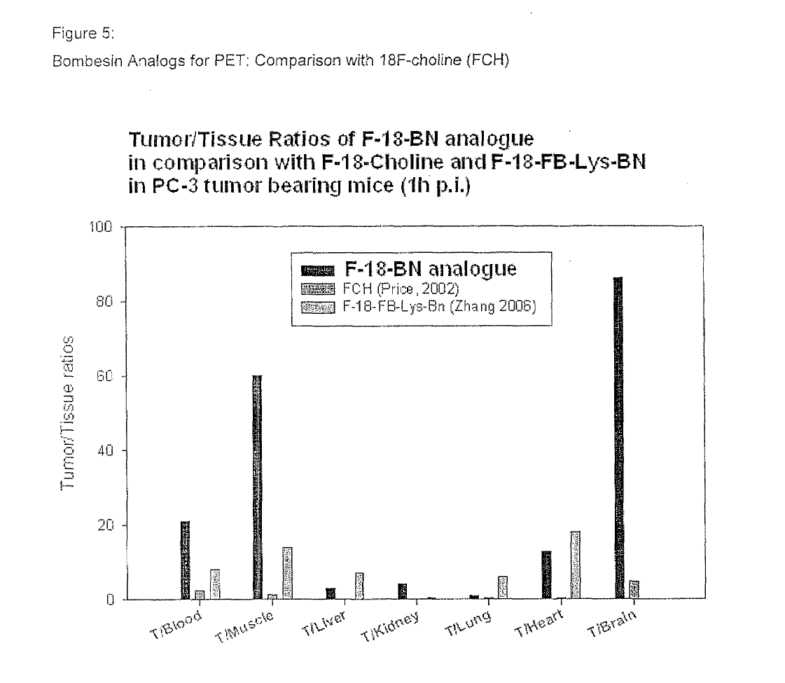

FIG. 4 shows radio- and UV-trace of the reaction according to FIG. 2 coinjected with the F-19 fluoro standard [¹⁹F]-2a.

FIGS. 1 and 2 are superposable for the F-18-2a pic. The same is observed for FIGS. 3 and 4.

### Biodistribution of F-18-Bombesin Analog

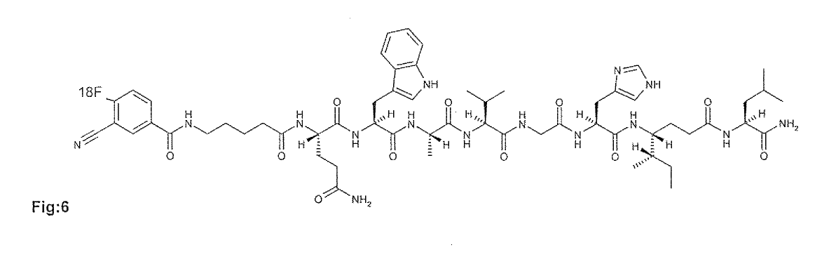

FIG. 6:

wherein Bombesin analogue is Gln-Trp-Ala-Val-Gly-His-FA01010-Leu-NH2

Radiolabeling of this bombesin analogue with F-18 was carried out via the method. The radiochemical yield was approx. 27% (decay corrected) giving 76 MBq in 50 μl ethanol with a radiochemical purity of >99% by HPLC and a specific activity of ˜480 GBq/mmol.

Nude mice bearing human prostate cancer PC-3 were injected with 100 μl radioactive peptide dissolved in PBS containing 135 kBq per animal. For blocking 100 μg unlabeled gastrin-releasing peptide was co-injected. One hour post injection the animals were sacrificed and organs dissected for counting in a gamma-counter. Values are expressed as percent of the injected dose per gram organ weight.

| | | 1 h |
|---|---|---|
| | 1 h | Blocking |
| | % ID/g | % ID/g |
| Tumor (% ID/g) | 1.00 ± 0.01 | 0.18 ± 0.03 |
| Blood (% ID/g | 0.05 ± 0.01 | 0.12 ± 0.00 |
| Muscle (% ID/g | 0.02 ± 0.00 | 0.03 ± 0.02 |
| Pancreas (% ID/g | 0.34 ± 0.03 | 0.10 ± 0.02 |
| Liver (% ID/g | 0.35 ± 0.13 | 0.39 ± 0.05 |
| Kidneys (% ID/g | 0.24 ± 0.02 | 0.71 ± 0.12 |
| Tumor/Tissue- | | |
| Ratios | | |
| T/Blood | 21.03 ± 11.92 | 1.57 ± 0.22 |
| T/Muscle | 59.99 ± 29.53 | 6.31 ± 3.27 |

It can be seen that ¹⁸F-labelled bombesin analog accumulates in tumor and the targeting agent ¹⁸F-labelled bombesin is specific since the blocking values are low in case of tumor and inchanged for the other tissue.

### Comparison of ¹⁸F-Labelled Bombesin Analogs

Protocol as above

### Table 1

Table 1 shows biodistribution in Nude mice bearing human prostate cancer PC-3 were injected with 100 μl radioactive peptide dissolved in PBS containing 135 kBq per animal.

Bombesin Analogs for PET: Comparison with 18F-Choline (FCH) and 18F-FB-Lys-BN

FIG. 5 shows that tumor-tissue ratio of Bombesin analog Gln-Trp-Ala-Val-Gly-His-FA01010-Leu-NH2 is 2.5 time higher than the tumor-tissue ratio of 18F-choline (FCH) and 18F-FB-Lys-BN.

Synthesis of H—Y-E: Solid-phase peptide synthesis (SPPS) involves the stepwise addition of amino acid residues to a growing peptide chain that is linked to an insoluble support or matrix, such as polystyrene. The C-terminal residue of the peptide is first anchored to a commercially available support (e.g., Rink amide resin) with its amino group protected with an N-protecting agent, fluorenylmethoxycarbonyl (FMOC) group. The amino protecting group is removed with suitable deprotecting agent such as piperidine for FMOC and the next amino acid residue (in N-protected form) is added with a coupling agents such as dicyclohexylcarbodiimide (DCC), di-isopropyl-cyclohexylcarbodiimide (DCCl), hydroxybenzotriazole (HOBt). Upon formation of a peptide bond, the reagents are washed from the support. After addition of the final residue of (Y), the peptide is attached to the solid support is ready for the coupling of RG-L₁-B₁—OH.

It is understood that the examples and embodiments described herein are for illustrative purpose only and that various modifications and changes in light thereof as well as combinations of features described in this application will be suggested to persons skilled in the art and are to be included within the spirit and purview of the described invention and within the scope of the appended claims. From the foregoing description, one skilled in the art can easily ascertain the essential characteristics of this invention and, without departing from the spirit and scope thereof, can make various changes and modifications of the invention to adapt it to various usages and conditions. The entire disclosure[s] of all applications, patents and publications, cited herein are incorporated by reference herein.

TABLE 1

| | Bind. Affinity | | Tumor % | | | | |
|---|---|---|---|---|---|---|---|
| Peptide sequence | (IC50) | | ID/g | Panc. % ID/g | Blocking | T/B | T/M |
| 3-CN,4-F-Bz-Ava-Gln-Trp-Ala-Val- | 6-10 | nM | 1 | 0.34 | >70% | 21.03 | 59.99 |
| Gly-His-FA01010-Leu-NH2 | | | | | | | |
| 3-CN,4-F-Benzoyl-Arg-Ava-Gln-Trp- | 1.9-2.7 | nM | 1.8 | 1.3 | 40-70% | 6.82 | 12.75 |
| Ala-Val-NMeGly-His-Sta-Leu-NH2 | | | | | | | |
| 3-CN,4-F-Benzoyl-Arg-Ava-Gln-Trp- | 1 | nM | 1.38 | 4.16 | 30-90% | 5.65 | 13.84 |
| Ala-Val-Gly-His(3Me)-Sta-Leu-NH2 | | | | | | | |
| 3-CF3,4-F-Benzoyl-Arg-Ava-Gln-Trp- | 0.3-1.8 | nM | 1.28 | 1.42 | >70% | 4.56 | 25.3 |
| Ala-Val-NMeGly-His-Sta-Leu-NH2 | | | | | | | |
| 4F,3CN-Bnz-Arg-Ava-Gln-Trp-Ala- | 2.3 | nM | 1.59 | 3.51 | 50-80% | 2.57 | 16.77 |
| Val-NMeGly-His(3Me)-Sta-Leu-NH2 | | | | | | | |

1. Compound having a general chemical Formula A: 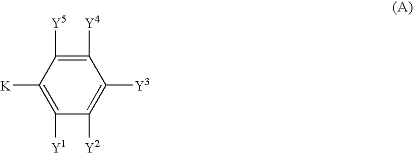
wherein
one of —Y¹, —Y², —Y³, —Y⁴ and —Y⁵ is a First Substituent (-G) which is selected from the group comprising —H, —F, —Cl, —Br, —I, —NO, —NO₂, —NR⁴COCF₃, —NR⁴SO₂CF₃, —N(CF₃)₂, —NHCSNHR⁴, —N(SO₂R⁵)₂, —N(O)═NCONH₂, —NR⁴CN, —NHCSR⁵, —N≡C, —N═C(CF₃)₂, —N═NCF₃, —N═NCN, —NR⁴COR⁴, —NR⁴COOR⁵, —OSO₂CF₃, —OSO₂C₆H₅, —OCOR⁵, —ONO₂, —OSO₂R⁵, —O—C═CH₂, —OCF₂CF₃, —OCOCF₃, —OCN, —OCF₃, —C≡N, —C(NO₂)₃, —COOR⁴, —CONR⁴R⁵, —C(S)NH₂, —CH═NOR⁴, —CH₂SO₂R⁴, —COCF₃, —CF₃, —CF₂Cl—CBr₃, —CClF₂, —CCl₃, —CF₂CF₃, —C≡CR⁴, —CH═NSO₂CF₃, —CH₂CF₃, —COR⁵, —CH═NOR⁵, —CH₂CONH₂, —CSNHR⁵, —CH═NNHCSNH₂, —CH═NNHCONHNH₂, —C≡C—CF₃, —CF═CFCF₃, —CF₂—CF₂—CF₃, —CR⁴(CN)₂, —COCF₂CF₂CF₃, —C(CF₃)₃, —C(CN)₃, —CR⁴═C(CN)₂, −1-pyrryl, —C(CN)═C(CN)₂, —C-pyridyl, —COC₆H₅, —COOC₆H₅, —SOCF₃, —SO₂CF₃, —SCF₃, —SO₂CN, —SCOCF₃, —SOR⁵, —S(OR⁵), —SC≡CR⁴, —SO₂R⁵, —SSO₂R⁵, —SR⁵, —SSR⁴, —SO₂CF₂CF₃, —SCF₂CF₃, —S(CF₃)═NSO₂CF₃, —SO₂C₆H₅, —SO₂N(R⁵)₂, —SO₂C(CF₃)₃, —SC(CF₃)₃, —SO(CF₃)═NSO₂CF₃, —S(O)(═NH)CF₃, —S(O)(═NH)R⁵, —S—C═CH₂, —SCOR⁵, —SOC₆H₅, —P(O)C₃F₇, —PO(OR⁵)₂, —PO(N(R⁵)₂)₂, —P(N(R⁵)₂)₂, —P(O)R⁵₂, and —PO(OR⁵)₂ and electron-withdrawing groups wherein the respective substituent can be in ortho, para or meta position in respect of the K (LG-O) group;
at least one of —Y¹, —Y², —Y³, —Y⁴ and —Y⁵ are Further Substituents (-Q) which are independently from each other selected from the group comprising —H, —CN, -halogen, —CF₃, —NO₂, —COR⁵ and —SO₂R⁵ wherein the respective substituent can be in ortho, para or meta position in respect of the K (LG-O),
wherein
R⁴ is hydrogen or a linear or branched C₁-C₆ alkyl,
R⁵ is hydrogen or a linear or branched C₁-C₆ alkyl,
wherein further one of —Y¹, —Y², —Y³, —Y⁴ and —Y⁵ is -A-B-D-P,
wherein
-A-B-D- is a bond or spacer,
P is a targeting agent, and
K is LG-O or W,
wherein:
LG is a leaving group, suitable for displacement by means of a nucleophilic aromatic substitution reaction and
W is a fluorine isotope (F),
as well as any pharmaceutically acceptable salts or organic or inorganic acids, hydrates, esters, amides, solvates and prodrugs thereof.

2. Compound according to claim 1, wherein W is radioactive or non-radioactive isotope of fluorine, more preferably ¹⁸F.

3. Compound according to any one of the preceding claims, wherein LG- is selected from the group comprising 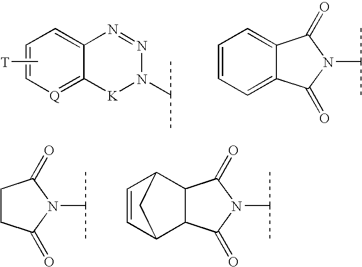
wherein
T is H or Cl,
Q is CH or N,
K is absent or is C═O.

4. Compound according to any one of the preceding claims, wherein LG- is selected from the group comprising

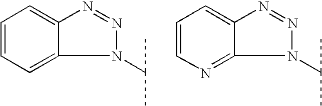

5. Compound according to any one of the preceding claims, wherein the First Substituent (-G) is selected from the group comprising —H, —F, —Cl, —Br, —NO₂, —OSO₂R⁵, —OCF₃, —C≡N, —COOR⁴, —CONR⁴R⁵, —COCF₃, —CF₂CF₃, —COR⁵, —CF₃, —C≡CF₃, —CF₂—CF₂—CF₃, —COC₆H₅, —SO₂CF₃, —SCOCF₃, —SO₂R⁵, —SO₂CF₂CF₃, —SO₂C₆H₅, —SO₂N(R⁵)₂, and —PO(OR⁵)₂ wherein the respective substituent can be in ortho, para or meta position in respect of the K (LG-O) group.

6. Compound according to any one of the preceding claims, wherein the Further Substituents (-Q) may independently from each other be selected from the group comprising —H, —CN, —F, —Cl, —Br and —NO₂, wherein the respective substituent can be in ortho, para or meta position in respect of the K (LG-O) group.

7. Compound according to any one of the preceding claims, wherein any of the First Substituent and said Further Substituents are independently from each other selected from the group comprising —H, —CN, —F, —Cl, —CF₃, —NO₂, —COCH₃ and —SO₂CH₃ wherein the respective substituent can be in ortho, para or meta position in respect of the K (LG-O) group.

8. Compound according to any one of the preceding claims, wherein any of the First Substituent and said Further Substituents are independently from each other selected from the group comprising —H, —CN and —Cl wherein the respective substituent can be in ortho, para or meta position in respect of the K (LG-O) group.

9. Compound according to any one of the preceding claims, wherein one of Y¹ and Y⁵ is selected from the group comprising CN and Cl wherein the respective substituent can be in ortho, para or meta position in respect of the K (LG-O) group.

10. Compound according to any one of the preceding claims, wherein R⁴ is hydrogen or linear or branched C₁-C₄ alkyl, R⁵ is hydrogen or linear or branched C₁-C₄ alkyl.

11. Compound according to any one of the preceding claims, wherein -A- is selected from the group comprising be selected from the group comprising a bond, —CO—, —SO₂—, —(CH₂)_{d}—CO—, —SO—, —C≡C—CO—, —[CH₂]ₘ-E-[CH₂]ₙ—CO—, —[CH₂]ₘ-E-[CH₂]ₙ—SO₂—, —C(═O)—O—, —NR¹⁰—, —O—, —(S)ₚ—, —C(═O)NR¹²—, —NR¹²—, —C(═S)NR¹²—, —C(═S)O—, C₁-C₆ cycloalkyl, alkenyl, heterocycloalkyl, unsubstituted and substituted aryl, heteroaryl, aralkyl, heteroaralkyl, alkyloxy, aryloxy, aralkyloxy, —SO₂NR¹³—, —NR¹³SO₂—, —NR¹³C(═O)O—, —NR¹³C(═O)NR¹²—, —NH—NH— and —NH—O—,
wherein
d is an integer of from 1 to 6,
m and n, independently, are any integer of from 0 to 5;
-E- is a bond, —S—, —O— or —NR⁹—,
wherein R⁹ is H, C₁-C₁₀ alkyl, aryl, heteroaryl or aralkyl,
p is any integer of from 1 to 3;
R¹⁰ and R¹², independently, are H, C₁-C₁₀ alkyl, aryl, heteroaryl or aralkyl and R¹³ is H, substituted or non substituted, linear or branched C₁-C₆ alkyl, aryl, cycloalkyl, heterocycloalkyl, aryl, heteroaryl, aralkyl or heteroaralkyl-.
further:
—B— is —NH— or —NR′—,
wherein R′ is a branched, cyclic or linear C₁-C₆ alkyl group.
and
-D- is —(CH₂)ₚ—CO— wherein p being an integer of from 1 to 10 or —(CH₂—CH₂—O)_{q}—CH₂—CH₂—CO— with q being an integer of from 1 to 5,
or
—B-D- together is a bond, one amino acid residue, an amino acid sequence with two (2) to twenty (20) amino acid residues or a non-amino acid group.

12. Compound according to any one of the preceding claims, wherein -A- is selected from the group comprising —CO—, —SO₂— and —C≡C—CO—.

13. Compound according to any one of the preceding claims, wherein -A- is selected from the group comprising —CO— and —SO₂—.

14. Compound according to any one of the preceding claims, wherein B-D is a natural or unnatural amino acid sequence or a non-amino acid group.

15. Compound according to any one of claims 14, wherein B-D is Arg-Ser, Arg-Ava, Lys(Me)2-β-ala, Lys(Me)-2-ser, Arg-β-ala, Ser-Ser, Ser-Thr, Arg-Thr, S-alkyl-cysteine, Cysteic acid, thioalkylcysteine (S—S-Alkyl) or 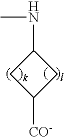
wherein k and l are independently selected in the range of from 0 to 4.

16. Compound according to any one of claims 14-15, wherein B-D is NH—(CH₂)ₚ—CO—, wherein p is an integer of from 1 to 10,
—NH—(CH₂—CH₂—O)_{q}—CH₂—CH₂—CO—, wherein q is an integer of from 1 to 5,
—NH-cycloalkyl-CO— wherein cycloalkyl is selected from C₅-C₈ cycloalkyl, or
—NH-heterocycloalkyl-(CH₂)ᵥ—CO— wherein heterocycloalkyl is selected from C₅-C₈ heterocycloalkyl containing carbon atoms and 1, 2, 3 or 4 oxygen, nitrogen or sulfur heteroatoms and v is an integer of from 1 to 4.

17. Compound according to any one of the preceding claims, wherein P is peptide, peptidomimetic, oligonucleotide or small molecule.

18. Compound according to any one of the preceding claims, wherein P is a peptide comprising from 4 to 100 amino acids.

19. Compound according to any one of the preceding claims, wherein P is selected from the group comprising bombesin, somatostatin receptor specific peptides, somatostatin, the derivatives and related peptides thereof, neuropeptide Y, neuropeptide Y₁, the derivatives and related peptides thereof, gastrin, gastrin releasing peptide, the derivatives and related peptides thereof, epidermal growth factor (EGF of various origin), insulin growth factor (IGF) and IGF-1, integrins (α₃β₁, αᵥβ₃, αᵥβ₅, αIIb₃), LHRH agonists and antagonists, transforming growth factors, particularly TGF-α, angiotensin, cholecystokinin receptor peptides, cholecystokinin (CCK) and the analogs thereof; neurotensin and the analogs thereof, thyrotropin releasing hormone, pituitary adenylate cyclase activating peptide (PACAP) and the related peptides thereof, chemokines, substrates and inhibitors for cell surface matrix metalloproteinase, prolactin and the analogs thereof, tumor necrosis factor, interleukins (IL-1, IL-2, IL-4 or IL-6), interferons, vasoactive intestinal peptide (VIP) and the related peptides thereof.

20. Compound according to any one of the preceding claims, wherein P is selected from the group comprising bombesin, somatostatin, neuropeptide Y₁ and analogs thereof.

21. Compound according to any one of the preceding claims, wherein P is selected from the group comprising bombesin analogs having a sequence of formula III or IV:
AA₁-AA₂-AA₃-AA₄-AA₅-AA₆-AA₇-AA₈-NT₁T₂ (type A) III,
with:
T₁=T₂=H or T₁=H,T₂═OH or T₁=CH₃, T₂═OH
AA₁=Gln, Asn, Phe(4-CO—NH₂)
AA₂=Trp, D-Trp
AA₃=Ala, Ser, Val
AA₄=Val, Ser, Thr
AA₅=Gly, (N-Me)Gly
AA₆=His, His(3-Me), (N-Me)His, (N-Me)His(3-Me)
AA₇═Sta, Statine analogs and isomers, 4-Am, 5-MeHpA, 4-Am, 5-MeHxA, γ-substituted aminoacids
AA₈=Leu, Cpa, Cba, CpnA, Cha, t-buGly, tBuAla, Met, Nle, iso-Bu-Gly
AA₁-AA₂-AA₃-AA₄-AA₅-AA₆-AA₇-AA₈-NT₁T₂ (type B) IV,
with:
T₁=T₂=H or T₁=H,T₂═OH or T₁=CH₃, T₂═OH
AA₁=Gln, Asn or Phe(4-CO—NH₂)
AA₂=Trp, D-Trp
AA₃=Ala, Ser, Val
AA₄=Val, Ser. Thr
AA₅=βAla, β² and β³-amino acids as shown herein after 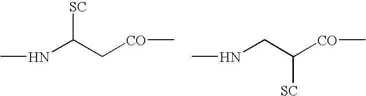
wherein SC represents a side chain found in proteinogenic amino acids and homologs of proteinogenic amino acids,
AA₆=His, His(3-Me), (N-Me)His, (N-Me)His(3-Me)
AA₇=Phe, Tha, NaI,
AA₈=Leu, Cpa, Cba, CpnA, Cha, t-buGly, tBuAla, Met, Nle, iso-Bu-Gly.

22. The compound according to any one of the preceding claims, wherein P is —NR⁷-peptide, or —(CH₂)ₙ-peptide, —O—(CH₂)ₙ— peptide or —S—(CH₂)ₙ— peptide, NR⁷— small-molecule, or —(CH₂)ₙ— small-molecule, —O—(CH₂)ₙ— small-molecule or —S—(CH₂)ₙ— small-molecule, NR⁷— oligonucleotide, or —(CH₂)ₙ— oligonucleotide, —O—(CH₂)ₙ— oligonucleotide or —S—(CH₂)ₙ— oligonucleotide, wherein n is an integer of from 1 to 6.

23. The compound according to any one of the preceding claims, wherein R⁷ is hydrogen or unbranched or branched C₁-C₆ alkyl.

24. The compound according to any one of the preceding claims, wherein R⁷ is hydrogen or methyl.

25. The compound according to any one of the preceding claims, wherein P is a small molecule having a molecular mass of from 200 to 800.

26. The compound according to any one of the preceding claims, wherein P is a oligonucleotide.

27. The compound according to any one of the preceding claims selected from

| | |
|---|---|
| 4-(Benzotriazol-1-yloxy)-3-cyano-benzoyl-valyl-β-alanyl-phenylalanyl-glycine amide, | (SEQ ID NO: 11) |
| 4-(Benzotriazol-1-yloxy)-3-cyano-benzoyl-valyl-β-alanyl-histidyl(π-Me)-glycine amide, | (SEQ ID NO: 13) |
| 3-cyano-4-([1,2,3]triazolo[4,5-b]pyridin-3-yloxy)-benzoyl-(5-aminopentanoyl)- | |
| phenylalanyl-(4(S)-amino-3(S)-hydroxy-6-methyl)heptanoyl-leucine amide, | |
| 4-(benzotriazol-1-yloxy)-3-chloro-benzoyl-valyl-β-alanyl-phenylalanyl-glycine amide, | (SEQ ID NO: 15) |
| 4-(Benzotriazol-1-yloxy)-3-cyano-benzoyl-Arg-Ava-Gln-Trp-Ala-Val-NMeGly-His(3Me)-Sta- | (SEQ ID NO: 16) |
| Leu-NH₂, | |
| 4-(Benzotriazol-1-yloxy)-3-cyano-benzoyl-1,4-cis-Achc-Gln-Trp-Ala-Val-Gly-His(3Me)- | (SEQ ID NO: 18) |
| Sta-Leu-NH₂, | |
| 4-(Benzotriazol-1-yloxy)-3-chloro-benzoyl-Gln-Trp-Ala-Val-Gly-His(3Me)-Sta-Leu-NH₂, | (SEQ ID NO: 19) |
| 4-(Benzotriazol-1-yloxy)-3-chloro-benzoyl-AOC-Gln-Trp-Ala-Val-Gly-His(3Me)-Sta-Leu- | (SEQ ID NO: 20) |
| NH₂, | |
| 4-(Benzotriazol-1-yloxy)-3-cyano-benzoyl-Ava-Gln-Trp-Ala-Val-NMeGly-His(3Me)-Sta- | (SEQ ID NO: 21) |
| Cpa-NH₂, | |
| 4-(Benzotriazol-1-yloxy)-3-cyano-benzoyl-Ava-Gln-Trp-Ala-Val-Gly-His(3Me)-FA4-Am,5- | (SEQ ID NO: 14) |
| MeHpA-Leu-NH₂, | |
| 3-Cyano-4-(2,5-dioxo-pyrrolidin-1-yloxy)-benzoyl-Ava-Gln-Trp-Ala-Val-Gly-His(3Me)-Sta- | (SEQ ID NO: 24) |
| Leu-NH₂, | |
| 3-Cyano-4-(2,5-dioxo-pyrrolidin-1-yloxy)-benzoyl-Arg-Ava-Gln-Trp-Ala-Val-Gly-His(3Me)- | (SEQ ID NO: 25) |
| Sta-Leu-NH₂, | |
| 3-Chloro-4-(2,5-dioxo-pyrrolidin-1-yloxy)-benzoyl-Arg-Ava-Gln-Trp-Ala-Val-Gly- | (SEQ ID NO: 26) |
| His(3Me)-Sta-Leu-NH₂, | |
| 3-Chloro-4-(2,5-dioxo-pyrrolidin-1-yloxy)-benzoyl-Ava-Gln-Trp-Ala-Val-Gly-His(3Me)- | (SEQ ID NO: 29) |
| Sta-Leu-NH₂, | |

3-Cyano-4-(2,5-dioxo-pyrrolidin-1-yloxy)-N-(thymidinyl-propyl)-benzamide: 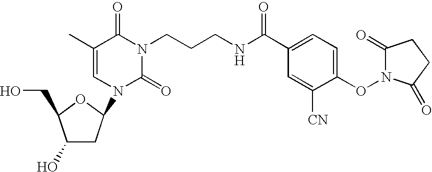
or
3-Cyano-4-(benzotriazol-1-yloxy)-N-(thymidinyl-propyl)-benzamide:

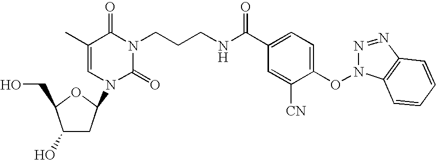

28. Compound according to any one of claims 1-26 comprising

| | |
|---|---|
| A-a-1: 4-[18]Fluoro-3-cyano-benzoyl-Arg-Ava-Gln-Trp-Ala-Val-NMeGly-His-Sta-Leu- | (SEQ ID NO: 31) |
| NH₂, | |
| A-a-2: 4-[18]Fluoro-3-cyano-benzoyl-Arg-Ava-Gln-Trp-Ala-Val-Gly-His(Me)-Sta-Leu- | (SEQ ID NO: 37) |
| NH₂, | |
| A-a-3: 4-[18]Fluoro-3-cyano-benzoyl-Arg-Ava-Gln-Trp-Ala-Val-NMeGly-His(3Me)- | (SEQ ID NO: 38) |
| Sta-Leu-NH₂, | |
| A-a-4: 4-[18]Fluoro-3-cyano-benzoyl-1,4-cis-Achc-Gln-Trp-Ala-Val-Gly-His(3Me)- | (SEQ ID NO: 39) |
| Sta-Leu-NH₂, | |
| A-a-5: 4-[18]Fluoro-3-cyano-benzoyl-Gln-Trp-Ala-Val-Gly-His(3Me)-Sta-Leu-NH₂, | (SEQ ID NO: 40) |
| A-a-6: 4-[18]Fluoro-3-cyano-benzoyl-AOC-Gln-Trp-Ala-Val-Gly-His(3Me)-Sta-Leu- | (SEQ ID NO: 41) |
| NH₂, | |
| A-a-7: 4-[18]Fluoro-3-cyano-benzoyl-Ava-Gln-Trp-Ala-Val-NMeGly-His(3Me)-Sta- | (SEQ ID NO: 44) |
| Cpa-NH₂, | |
| A-a-8: 4-[18]Fluoro-3-cyano-benzoyl-Ava-Gln-Trp-Ala-Val-Gly-His(3Me)-FA4-Am,5- | (SEQ ID NO: 45) |
| MeHpA-Leu-NH₂, | |
| A-a-9: 4-[18]Fluoro-3-cyano-benzoyl-Ava-Gln-Trp-Ala-Val-Gly-His(3Me)-Sta-Leu- | (SEQ ID NO: 47) |
| NH₂, | |
| A-a-10: 4-[18]Fluoro-3-cyano-benzoyl-Lys(Me)2-βAla-Gln-Trp-Ala-Val-Gly-His(3Me)- | (SEQ ID NO: 76) |
| Sta-Leu-NH₂, | |
| A-a-11: 4-[18]Fluoro-3-cyano-benzoyl-Lys(Me)2-βAla-Gln-Trp-Ala-Val-Gly-His(3Me)- | (SEQ ID NO: 76) |
| Sta-Leu-NH₂, | |
| A-a-12: 4-[18]Fluoro-3-cyano-benzoyl-Arg-Ser-Gln-Trp-Ala-Val-Gly-His(3Me)-4- | (SEQ ID NO: 78) |
| Am,5-MeHpA-Leu-NH₂, | |
| A-a-13: 4-[18]Fluoro-3-cyano-benzoyl-Ser-Ser-Gln-Trp-Ala-Val-Gly-His(3Me)-4- | (SEQ ID NO: 80) |
| Am,5-MeHpA-Leu-NH₂, | |
| A-a-14: 4-[18]Fluoro-3-cyano-benzoyl-Lys(Me)2-Ser-Gln-Trp-Ala-Val-Gly-His(3Me)- | (SEQ ID NO: 81) |
| 4-Am,5-MeHpA-Leu-NH₂, | |
| A-a--15: 4-[18]Fluoro-3-cyano-benzoyl-Arg-Ser-Gln-Trp-Ala-Val-Gly-His(3Me)-Sta- | (SEQ ID NO: 83) |
| Leu-NH₂, | |
| A-a-16: 4-[18]Fluoro-3-cyano-benzoyl-Lys(Me)2-βAla-Gln-Trp-Ala-Val-Gly-His(3Me)- | (SEQ ID NO: 84) |
| 4-Am,5-MeHpA-Leu-NH₂, | |
| A-a-17: 4-[18]Fluoro-3-cyano-benzoyl-Ava-Gln-Trp-Ala-Val-Gly-His-4-Am,5-MeHpA- | (SEQ ID NO: 85) |
| Leu-NH₂, | |
| A-a-18: 4-[18]Fluoro-3-trifluoromethyl-benzoyl-Arg-Ava-Gln-Trp-Ala-Val-Gly- | (SEQ ID NO: 86) |
| His(3Me)-Sta-LeuNH₂, | |
| A-a-19: 4-[18]Fluoro-3-trifluoromethyl-benzoyl-Arg-Ava-Gln-Trp-Ala-Val-NMeGly- | (SEQ ID NO: 87) |
| His(3Me)-Sta-Leu-NH₂, | |
| A-a-20: 4-[18]Fluoro-3-trifluoromethyl-benzoyl-1,4-cis-Achc-Gln-Trp-Ala-Val-Gly- | (SEQ ID NO: 88) |
| His(3Me)-Sta-Leu-NH₂, | |
| A-a-21: 4-[18]Fluoro-3-trifluoromethyl-benzoyl-Gln-Trp-Ala-Val-Gly-His(3Me)-Sta- | (SEQ ID NO: 92) |
| Leu-NH₂, | |
| A-a-22: 4-[18]Fluoro-3-trifluoromethyl-benzoyl-Arg-βAla-Gln-Trp-Ala-Val-Gly- | (SEQ ID NO: 93) |
| His(3Me)-4-Am,5-MeHpA-Leu-NH₂, | |
| IIB-a-23 4-[18]-Fluoro-3-cyano-benzoyl-Ava-Gln-Trp-Ala-Val-NMeGly-His(3Me)- | (SEQ ID NO: 94) |
| 4-Am,5-MeHpA-Cpa-NH₂, | |
| IIB-a-24 4-[18]-Fluoro-3-cyano-benzoyl-Ser-Ser-Gln-Trp-Ala-Val-Gly-His(3Me)- | (SEQ ID NO: 95) |
| Sta-Leu-NH₂, | |
| IIB-a-25 4-[18]-Fluoro-3-cyano-benzoyl-DOA-Gln-Trp-Ala-Val-Gly-His(3Me)- | (SEQ ID NO: 96) |
| Sta-Leu-NH₂, | |
| IIB-a-26 3,4-[18]-Difluorobenzoyl-Ava-Gln-Trp-Ala-Val-NMeGly-His-Sta-Leu- | (SEQ ID NO: 97) |
| NH₂, | |
| IIB-a-27 3,4-[18]-Difluorobenzoyl-Ava-Gln-Trp-Ala-Val-NMeGly-His-FA02010- | (SEQ ID NO: 98) |
| Cpa-NH₂, | |
| IIB-a-28 3,4-[18]-Difluorobenzoyl-Ava-Gln-Trp-Ala-Val-NMeGly-His-4-Am,5- | (SEQ ID NO: 99) |
| MeHpA-tbuGly-NH₂, | |
| IIB-a-29 3,4-[18]-Difluorobenzoyl-Ava-Gln-Trp-Ala-Val-NMeGly-His(3Me)-Sta- | (SEQ ID NO: 100) |
| Leu-NH₂, | |
| IIB-a-30 3,4-[18]-Difluorobenzoyl-Ava-Gln-Trp-Ala-Val-NMeGly-His(3Me)-Sta- | (SEQ ID NO: 103) |
| tBuGly-NH₂, | |
| IIB-a-31 3,4-[18]-Difluorobenzoyl-Ava-Gln-Trp-Ala-Val-Gly-His(3Me)-Sta-Leu- | (SEQ ID NO: 108) |
| NH₂, | |
| IIB-a-32 3,4-[18]-Difluorobenzoyl-Ava-Gln-Trp-Ala-Val-NMeGly-His(3Me)-4- | (SEQ ID NO: 109) |
| Am,5-MeHpA-Leu-NH₂, | |
| IIB-a-33 3,4-[18]-Difluorobenzoyl-Ava-Gln-DTrp-Ala-Val-Gly-His-4-Am,5- | |
| MeHpA-tbuGly-NH₂, | |
| IIB-a-34 3,4-[18]-Difluorobenzoyl-Ava-Gln-DTrp-Ala-Val-Gly-His-4-Am-5- | |
| MeHxA-Cpa-NH₂, | |
| IIB-a-35 3,4-[18]-Difluorobenzoyl-Ava-Gln-Trp-Ala-Val-NMeGly-His(3Me)-Sta- | (SEQ ID NO: 110) |
| Cpa-NH₂ | |
| IIB-a-36 3,4-[18]-Difluorobenzoyl-Ava-Gln-DTrp-Ala-Val-Gly-His-Sta-tbuAla- | |
| NH₂ , | |
| IIB-a-37 3,4-[18]-Difluorobenzoyl-Arg-Ava-Gln-Trp-Ala-Val-NMeGly-His-Sta- | (SEQ ID NO: 111) |
| Leu-NH₂ | |
| IIB-a-38 3,4-[18]-Difluorobenzoyl-Gln-Trp-Ala-Val-Gly-His(3Me)-Sta-Leu-NH₂ | (SEQ ID NO: 112) |
| IIB-a-39 3,4-[18]-Difluorobenzoyl-Arg-Ava-Gln-Trp-Ala-Val-NMeGly-His(3Me)- | (SEQ ID NO: 113) |
| Sta-Leu-NH₂ | |
| IIB-a-40 3,4-[18]-Difluorobenzoyl-Arg-Ava-Gln-Trp-Ala-Val-Gly-His(3Me)-Sta- | (SEQ ID NO: 114) |
| Leu-NH₂ | |
| IIB-a-41 3,4-[18]-Difluorobenzoyl-Arg-βAla-Arg-Gln-Trp-Ala-Val-Gly-His(3Me)- | (SEQ ID NO: 115) |
| Sta-Leu-NH₂ | |
| IIB-a-42 3,4-[18]-Difluorobenzoyl-Ava-Gln-Trp-Ala-Val-Gly-His(3Me)-Sta-Cpa- | (SEQ ID NO: 116) |
| NH₂ | |
| IIB-a-43 3,4-[18]-Difluorobenzoyl-Ava-Gln-Trp-Ala-Val-Gly-His(3Me)-Sta- | (SEQ ID NO: 117) |
| tBuGly-NH₂ | |
| IIB-a-44 3,4-[18]-Difluorobenzoyl-Arg-Arg-Gln-Trp-Ala-Val-NMeGly-His(3Me)- | (SEQ ID NO: 118) |
| Sta-Leu-NH₂ | |
| IIB-a-45 3,4-[18]-Difluorobenzoyl-Arg-βAla-Gln-Trp-Ala-Val-NMeGly-His(3Me)- | (SEQ ID NO: 119) |
| Sta-Leu-NH₂ | |
| IIB-a-46 3,4-[18]-Difluorobenzoyl-Gln-Trp-Ala-Val-Gly-His(3Me)-4-Am,5- | (SEQ ID NO: 120) |
| MeHpA-Leu-NH₂ | |
| IIB-a-47 3,4-[18]-Difluorobenzoyl-Ava-Gln-Trp-Ala-Val-NMeGly-His(3Me)-4- | (SEQ ID NO: 121) |
| Am,5-MeHpA-Cpa-NH₂ | |
| IIB-a-48 3,4-[18]-Difluorobenzoyl-Ava-Gln-Trp-Ala-Val-Gly-His(3Me)-4-Am,5- | (SEQ ID NO: 122) |
| MeHpA-Leu-NH₂ | |
| IIB-a-49 3,4-[18]-Difluorobenzoyl-Ava-Gln-Trp-Ala-Val-Gly-NMeHis-4-Am,5- | (SEQ ID NO: 123) |
| MeHpA-Cpa-NH₂, | |
| IIB-a-49 3,4-[18]-Difluorobenzoyl-Ava-Gln-Trp-Ala-Val-Gly-NMeHis(3Me)-4- | (SEQ ID NO: 124) |
| Am,5-MeHpA-Leu-NH₂, | |
| IIB-a-50 3,4-[18]-Difluorobenzoyl-Ava-Gln-Trp-Ala-Val-Gly-NMeHis-4-Am,5- | (SEQ ID NO: 125) |
| MeHpA-Leu-NH₂, | |
| IIB-a-51 3,4-[18]-Difluorobenzoyl-Ava-Gln-Trp-Ala-Val-NMeGly-His-AHMHxA- | (SEQ ID NO: 126) |
| Leu-NH₂, | |
| IIB-a-52 3,4-[18]-Difluorobenzoyl-Ava-Gln-Trp-Ala-Val-βAla-NMeHis-Tha-Cpa- | (SEQ ID NO: 127) |
| NH₂, | |
| IIB-a-53 3,4-[18]-Difluorobenzoyl-Ava-Gln-Trp-Ala-Val-βAla-NMeHis-Phe-Cpa- | (SEQ ID NO: 128) |
| NH₂, | |
| IIB-a-54 3,4-[18]-Difluorobenzoyl-Ava-Gln-Trp-Ala-Val-βAla-NMeHis-Phe-Leu- | (SEQ ID NO: 129) |
| NH₂, | |
| IIB-a-55 3,4-[18]-Difluorobenzoyl-Ava-Gln-Trp-Ala-Val-βAla-DHis-Phe-Leu-NH₂, | |
| IIB-a-56 3,4-[18]-Difluorobenzoyl-Ava-Gln-Trp-Ala-Val-βAla-His-βhLeu-Leu- | (SEQ ID NO: 130) |
| NH₂, | |
| IIB-a-57 3,4-[18]-Difluorobenzoyl-Ava-Gln-Trp-Ala-Val-βAla-His-βhIle-Leu-NH₂, | (SEQ ID NO: 131) |
| IIB-a-58 3,4-[18]-Difluorobenzoyl-Ava-Gln-Trp-Ala-Val-βAla-His-βhLeu-tbuGly- | (SEQ ID NO: 132) |
| NH₂, | |
| IIB-a-59 3,4-[18]-Difluorobenzoyl-Ava-Gln-Trp-Ala-Val-βAla-His(3Me)-Phe-Tha- | (SEQ ID NO: 133) |
| NH₂, | |
| IIB-a-60 3,4-[18]-Difluorobenzoyl-Ava-Gln-Trp-Ala-Val-βAla-His(3Me)-Phe-Nle- | (SEQ ID NO: 134) |
| NH₂, | |
| IIB-a-61 3,4-[18]-Difluorobenzoyl-Ava-Gln-Trp-Ala-Val-βAla-NMeHis-Phe- | (SEQ ID NO: 135) |
| tbuGly-NH₂, | |
| IIB-a-62 3,4-[18]-Difluorobenzoyl-Ava-Gln-Trp-Ala-Val-βAla-NMeHis-Tha- | (SEQ ID NO: 136) |
| tbuGly-NH₂, | |
| IIB-a-63 3,4-[18]-Difluorobenzoyl-Ava-Gln-Trp-Ala-Val-βAla-His(3Me)-Tha- | (SEQ ID NO: 137) |
| tbuGly-NH₂, | |
| IIB-a-64 3,4-[18]-Difluorobenzoyl-Ava-Gln-Trp-Ala-Val-βAla-His(3Me)-Phe-Cpa- | (SEQ ID NO: 138) |
| NH₂, | |
| IIB-a-65 3,4-[18]-Difluorobenzoyl-Ava-Gln-Trp-Ala-NMeVal-βAla-His-Phe-Leu- | (SEQ ID NO: 139) |
| NH₂, | |
| IIB-a-66 3,4-[18]-Difluorobenzoyl-Ava-Gln-Trp-Ala-Val-βAla-His-NMePhe-Leu- | (SEQ ID NO: 140) |
| NH₂, | |
| IIB-a-67 3,4-[18]-Difluorobenzoyl-Ava-Gln-DTrp-Ala-Val-βAla-His-Phe-Leu-NH₂, | |
| IIB-a-68 3,4-[18]-Difluorobenzoyl-Ava-Gln-Trp-DAla-Val-βAla-His-Phe-Leu-NH₂, | |
| IIB-a-69 3,4-[18]-Difluorobenzoyl-Ava-Gln-Trp-Ala-DVal-βAla-His-Phe-Leu-NH₂, | |
| IIB-a-70 3,4-[18]-Difluorobenzoyl-Ava-Gln-Trp-Ala-Val-βAla-His-DPhe-Leu-NH₂, | |
| IIB-a-71 3,4-[18]-Difluorobenzoyl-Ava-Gln-Trp-Ala-Val-βAla-His-βIle-tbuGly- | (SEQ ID NO: 141) |
| NH₂, | |
| IIB-a-72 4-[18]-Fluoro-3-cyano-phenylsulfonyl-Ava-Gln-Trp-Ala-Val-NMeGly- | (SEQ ID NO: 142) |
| His-4-Am,5-MeHpA-Cpa-NH₂, | |
| IIB-a-73 4-[18]-Fluoro-3-cyano-phenylsulfonyl-Ava-Gln-Trp-Ala-Val-NMeGly- | (SEQ ID NO: 143) |
| His-Sta-Cpa-NH₂, | |
| IIB-a-74 4-[18]-Fluoro-3-cyano-phenylsulfonyl-Ava-Gln-Trp-Ala-Val-NMeGly- | (SEQ ID NO: 144) |
| His-Sta-tbuAla-NH₂, | |
| IIB-a-75 4-[18]-Fluoro-3-cyano-phenylsulfonyl-Ava-Gln-Trp-Ala-Val-NMeGly- | (SEQ ID NO: 145) |
| His-4-Am,5-MeHpA-tbuAla-NH₂, | |
| 4-[18]Fluoro-3-cyano-benzoyl-(piperidyl-4-carbonyl)-Gln-Trp-Ala-Val-Gly-His(3Me)- | (SEQ ID NO: 146) |
| Sta-Leu-NH₂, | |
| 4-[18]Fluoro-3-cyano-benzoyl-(piperazin-1-yl-acetyl)-Gln-Trp-Ala-Val-Gly-His(3Me)- | (SEQ ID NO: 147) |
| Sta-Leu-NH₂, | |
| 4-[18]Fluoro-3-cyano-benzoyl-1,4-trans-Achc-Gln-Trp-Ala-Val-NMeGly-His-Sta-Leu- | (SEQ ID NO: 148) |
| NH₂, | |
| B-a-1: 4-[19]-Fluoro-3-cyano-benzoyl-Arg-Ava-Gln-Trp-Ala-Val-NMeGly-His-Sta- | (SEQ ID NO: 149) |
| Leu-NH₂, | |
| B-a--2: 4-[19]-Fluoro-3-cyano-benzoyl-Arg-Ava-Gln-Trp-Ala-Val-His(Me)-Sta-Leu- | (SEQ ID NO: 150) |
| NH₂, | |
| B-a-3: 4-[19]-Fluoro-3-cyano-benzoyl-Arg-Ava-Gln-Trp-Ala-Val-NMeGly-His(3Me)- | (SEQ ID NO: 151) |
| Sta-Leu-NH₂, | |
| B-a-4: 4-[19]-Fluoro-3-cyano-benzoyl-1,4-cis-Achc-Gln-Trp-Ala-Val-Gly-His(3Me)- | (SEQ ID NO: 152) |
| Sta-Leu-NH₂, | |
| B-a-5: 4-[19]-Fluoro-3-cyano-benzoyl-Gln-Trp-Ala-Val-Gly-His(3Me)-Sta-Leu-NH₂, | (SEQ ID NO: 153) |
| B-a-6: 4-[19]-Fluoro-3-cyano-benzoyl-AOC-Gln-Trp-Ala-Val-Gly-His(3Me)-Sta-Leu- | (SEQ ID NO: 154) |
| NH₂, | |
| B-a-7: 4-[19]-Fluoro-3-cyano-benzoyl-Ava-Gln-Trp-Ala-Val-NMeGly-His(3Me)-Sta- | (SEQ ID NO: 155) |
| Cpa-NH₂, | |
| B-a-8: 4-[19]-Fluoro-3-cyano-benzoyl-Ava-Gln-Trp-Ala-Val-Gly-His(3Me)-4-Am,5- | (SEQ ID NO: 156) |
| MeHpA-Leu-NH₂, | |
| B-a-9: 4-[19]-Fluoro-3-cyano-benzoyl-Ava-Gln-Trp-Ala-Val-Gly-His(3Me)-Sta-Leu- | (SEQ ID NO: 157) |
| NH₂, | |
| B-a-10: 4-[19]-Fluoro-3-cyano-benzoyl-Lys(Me)2-βAla-Gln-Trp-Ala-Val-Gly- | (SEQ ID NO: 158) |
| His(3Me)-Sta-Leu-NH₂, | |
| B-a-11: 4-[19]-Fluoro-3-cyano-benzoyl-Lys(Me)2-βAla-Gln-Trp-Ala-Val-Gly- | (SEQ ID NO: 159) |
| His(3Me)-Sta-Leu-NH₂, | |
| B-a-12: 4-[19]-Fluoro-3-cyano-benzoyl-Arg-Ser-Gln-Trp-Ala-Val-Gly-His(3Me)-4- | (SEQ ID NO: 160) |
| Am,5-MeHpA-Leu-NH₂, | |
| B-a-13: 4-[19]-Fluoro-3-cyano-benzoyl-Ser-Ser-Gln-Trp-Ala-Val-Gly-His(3Me)-4- | (SEQ ID NO: 161) |
| Am,5-MeHpA-Leu-NH₂, | |
| B-a-14: 4-[19]-Fluoro-3-cyano-benzoyl-Lys(Me)2-Ser-Gln-Trp-Ala-Val-Gly-His(3Me)- | (SEQ ID NO: 162) |
| 4-Am,5-MeHpA-Leu-NH₂, | |
| B-a-15: 4-[19]-Fluoro-3-cyano-benzoyl-Arg-Ser-Gln-Trp-Ala-Val-Gly-His(3Me)-Sta- | (SEQ ID NO: 163) |
| Leu-NH₂, | |
| B-a-16: 4-[19]-Fluoro-3-cyano-benzoyl-Lys(Me)2-βAla-Gln-Trp-Ala-Val-Gly- | (SEQ ID NO: 164) |
| His(3Me)-4-Am,5-MeHpA-Leu-NH₂, | |
| B-a-17: 4-[19]-Fluoro-3-cyano-benzoyl-Ava-Gln-Trp-Ala-Val-Gly-His-4-Am,5-MeHpA- | (SEQ ID NO: 165) |
| Leu-NH₂, | |
| B-a-18: 4-[19]-Fluoro-3-trifluoromethyl-benzoyl-Arg-Ava-Gln-Trp-Ala-Val-Gly- | (SEQ ID NO: 166) |
| His(3Me)-Sta-Leu-NH₂, | |
| B-a-19: 4-[19]-Fluoro-3-trifluoromethyl-benzoyl-Arg-Ava-Gln-Trp-Ala-Val-NMeGly- | (SEQ ID NO: 167) |
| His(3Me)-Sta-Leu-NH₂, | |
| B-a-20: 4-[19]-Fluoro-3-trifluoromethyl-benzoyl-1,4-cis-Achc-Gln-Trp-Ala-Val-Gly- | (SEQ ID NO: 168) |
| His(3Me)-Sta-Leu-NH₂, | |
| B-a-21: 4-[19]-Fluoro-3-trifluoromethyl-benzoyl-Gln-Trp-Ala-Val-Gly-His(3Me)-Sta- | (SEQ ID NO: 169) |
| Leu-NH₂, | |
| B-a-22: 4-[19]-Fluoro-3-trifluoromethyl-benzoyl-Arg-βAla-Gln-Trp-Ala-Val-Gly- | (SEQ ID NO: 170) |
| His(3Me)-4-Am,5-MeHpA-Leu-NH₂, | |
| B-a-23: 4-[19]-Fluoro-3-cyano-benzoyl-Ava-Gln-Trp-Ala-Val-NMeGly-His(3Me)-4- | (SEQ ID NO: 171) |
| Am,5-MeHpA-Cpa-NH₂, | |
| B-a-24: 4-[19]-Fluoro-3-cyano-benzoyl-Ser-Ser-Gln-Trp-Ala-Val-Gly-His(3Me)-Sta- | (SEQ ID NO: 172) |
| Leu-NH₂, | |
| B-a-25: 4-[19]-Fluoro-3-cyano-benzoyl-DOA-Gln-Trp-Ala-Val-Gly-His(3Me)-Sta-Leu- | (SEQ ID NO: 173) |
| NH₂, | |
| B-a-26: 3,4-[19]-Difluorobenzoyl-Ava-Gln-Trp-Ala-Val-NMeGly-His-Sta-Leu-NH₂, | (SEQ ID NO: 174) |
| B-a-27: 3,4-[19]-Difluorobenzoyl-Ava-Gln-Trp-Ala-Val-NMeGly-His-FA02010-Cpa- | (SEQ ID NO: 175) |
| NH₂, | |
| B-a-28: 3,4-[19]-Difluorobenzoyl-Ava-Gln-Trp-Ala-Val-NMeGly-His-4-Am,5-MeHpA- | (SEQ ID NO: 176) |
| tbuGly-NH₂, | |
| B-a-29: 3,4-[19]-Difluorobenzoyl-Ava-Gln-Trp-Ala-Val-NMeGly-His(3Me)-Sta-Leu- | (SEQ ID NO: 177) |
| NH₂, | |
| B-a-30: 3,4-[19]-Difluorobenzoyl-Ava-Gln-Trp-Ala-Val-NMeGly-His(3Me)-Sta-tBuGly- | (SEQ ID NO: 178) |
| NH₂, | |
| B-a-31: 3,4-[19]-Difluorobenzoyl-Ava-Gln-Trp-Ala-Val-Gly-His(3Me)-Sta-Leu-NH₂, | (SEQ ID NO: 179) |
| B-a-32: 3,4-[19]-Difluorobenzoyl-Ava-Gln-Trp-Ala-Val-NMeGly-His(3Me)-4-Am,5- | (SEQ ID NO: 180) |
| MeHpA-Leu-NH₂, | |
| B-a-33: 3,4-[19]-Difluorobenzoyl-Ava-Gln-DTrp-Ala-Val-Gly-His-4-Am,5-MeHpA- | |
| tbuGly-NH₂, | |
| B-a-34: 3,4-[19]-Difluorobenzoyl-Ava-Gln-DTrp-Ala-Val-Gly-His-4-Am-5-MeHxA- | |
| Cpa-NH₂, | |
| B-a-35: 3,4-[19]-Difluorobenzoyl-Ava-Gln-Trp-Ala-Val-NMeGly-His(3Me)-Sta-Cpa- | (SEQ ID NO: 181) |
| NH₂, | |
| B-a-36: 3,4-[19]-Difluorobenzoyl-Ava-Gln-DTrp-Ala-Val-Gly-His-Sta-tbuAla-NH₂, | |
| B-a-37: 3,4-[19]-Difluorobenzoyl-Arg-Ava-Gln-Trp-Ala-Val-NMeGly-His-Sta-Leu-NH₂, | (SEQ ID NO: 182) |
| B-a-38: 3,4-[19]-Difluorobenzoyl-Gln-Trp-Ala-Val-Gly-His(3Me)-Sta-Leu-NH₂, | (SEQ ID NO: 183) |
| B-a-39: 3,4-[19]-Difluorobenzoyl-Arg-Ava-Gln-Trp-Ala-Val-NMeGly-His(3Me)-Sta- | (SEQ ID NO: 184) |
| Leu-NH₂, | |
| B-a-40: 3,4-[19]-Difluorobenzoyl-Arg-Ava-Gln-Trp-Ala-Val-Gly-His(3Me)-Sta-Leu- | (SEQ ID NO: 185) |
| NH₂, | |
| B-a-41: 3,4-[19]-Difluorobenzoyl-Arg-βAla-Arg-Gln-Trp-Ala-Val-Gly-His(3Me)-Sta-Leu- | (SEQ ID NO: 186) |
| NH₂, | |
| B-a-42: 3,4-[19]-Difluorobenzoyl-Ava-Gln-Trp-Ala-Val-Gly-His(3Me)-Sta-Cpa-NH₂, | (SEQ ID NO: 187) |
| B-a-43: 3,4-[19]-Difluorobenzoyl-Ava-Gln-Trp-Ala-Val-Gly-His(3Me)-Sta-tBuGly-NH₂, | (SEQ ID NO: 188) |
| B-a-44: 3,4-[19]-Difluorobenzoyl-Arg-Arg-Gln-Trp-Ala-Val-NMeGly-His(3Me)-Sta- | (SEQ ID NO: 189) |
| Leu-NH₂, | |
| B-a-45: 3,4-[19]-Difluorobenzoyl-Arg-βAla-Gln-Trp-Ala-Val-NMeGly-His(3Me)-Sta- | (SEQ ID NO: 190) |
| Leu-NH₂, | |
| B-a-46: 3,4-[19]-Difluorobenzoyl-Gln-Trp-Ala-Val-Gly-His(3Me)-4-Am,5-MeHpA-Leu- | (SEQ ID NO: 191) |
| NH₂, | |
| B-a-47: 3,4-[19]-Difluorobenzoyl-Ava-Gln-Trp-Ala-Val-NMeGly-His(3Me)-4-Am,5- | (SEQ ID NO: 192) |
| MeHpA-Cpa-NH₂, | |
| B-a-48: 3,4-[19]-Difluorobenzoyl-Ava-Gln-Trp-Ala-Val-Gly-His(3Me)-4-Am,5-MeHpA- | (SEQ ID NO: 193) |
| Leu-NH₂, | |
| B-a-49: 3,4-[19]-Difluorobenzoyl-Ava-Gln-Trp-Ala-Val-Gly-NMeHis-4-Am,5-MeHpA- | (SEQ ID NO: 194) |
| Cpa-NH₂, | |
| B-a-49: 3,4-[19]-Difluorobenzoyl-Ava-Gln-Trp-Ala-Val-Gly-NMeHis(3Me)-4-Am,5- | (SEQ ID NO: 195) |
| MeHpA-Leu-NH₂, | |
| B-a-50: 3,4-[19]-Difluorobenzoyl-Ava-Gln-Trp-Ala-Val-Gly-NMeHis-4-Am,5-MeHpA- | (SEQ ID NO: 196) |
| Leu-NH₂, | |
| B-a-51: 3,4-[19]-Difluorobenzoyl-Ava-Gln-Trp-Ala-Val-NMeGly-Hls-AHMHxA-Leu- | (SEQ ID NO: 197) |
| NH₂, | |
| B-a-52: 3,4-[19]-Difluorobenzoyl-Ava-Gln-Trp-Ala-Val-βAla-NMeHis-Tha-Cpa-NH₂, | (SEQ ID NO: 198) |
| B-a-53: 3,4-[19]-Difluorobenzoyl-Ava-Gln-Trp-Ala-Val-βAla-NMeHis-Phe-Cpa-NH₂, | (SEQ ID NO: 199) |
| B-a-54: 3,4-[19]-Difluorobenzoyl-Ava-Gln-Trp-Ala-Val-βAla-NMeHis-Phe-Leu-NH₂, | (SEQ ID NO: 200) |
| B-a-55: 3,4-[19]-Difluorobenzoyl-Ava-Gln-Trp-Ala-Val-βAla-DHis-Phe-Leu-NH₂, | |
| B-a-56: 3,4-[19]-Difluorobenzoyl-Ava-Gln-Trp-Ala-Val-βAla-His-βhLeu-Leu-NH₂, | (SEQ ID NO: 201) |
| B-a-57: 3,4-[19]-Difluorobenzoyl-Ava-Gln-Trp-Ala-Val-βAla-His-βhIle-Leu-NH₂, | (SEQ ID NO: 202) |
| B-a-58: 3,4-[19]-Difluorobenzoyl-Ava-Gln-Trp-Ala-Val-βAla-His-βhLeu-tbuGly-NH₂, | (SEQ ID NO: 203) |
| B-a-59: 3,4-[19]-Difluorobenzoyl-Ava-Gln-Trp-Ala-Val-βAla-His(3Me)-Phe-Tha-NH₂, | (SEQ ID NO: 204) |
| B-a-60: 3,4-[19]-Difluorobenzoyl-Ava-Gln-Trp-Ala-Val-βAla-His(3Me)-Phe-Nle-NH₂, | (SEQ ID NO: 205) |
| B-a-61: 3,4-[19]-Difluorobenzoyl-Ava-Gln-Trp-Ala-Val-βAla-NMeHis-Phe-tbuGly-NH₂, | (SEQ ID NO: 206) |
| B-a-62: 3,4-[19]-Difluorobenzoyl-Ava-Gln-Trp-Ala-Val-βAla-NMeHis-Tha-tbuGly-NH₂, | (SEQ ID NO: 207) |
| B-a-63: 3,4-[19]-Difluorobenzoyl-Ava-Gln-Trp-Ala-Val-βAla-His(3Me)-Tha-tbuGly- | (SEQ ID NO: 208) |
| NH₂, | |
| B-a-64: 3,4-[19]-Difluorobenzoyl-Ava-Gln-Trp-Ala-Val-βAla-His(3Me)-Phe-Cpa-NH₂, | (SEQ ID NO: 209) |
| B-a-65: 3,4-[19]-Difluorobenzoyl-Ava-Gln-Trp-Ala-NMeVal-βAla-His-Phe-Leu-NH₂, | (SEQ ID NO: 210) |
| B-a-66: 3,4-[19]-Difluorobenzoyl-Ava-Gln-Trp-Ala-Val-βAla-His-NMePhe-Leu-NH₂, | (SEQ ID NO: 211) |
| B-a-67: 3,4-[19]-Difluorobenzoyl-Ava-Gln-DTrp-Ala-Val-βAla-His-Phe-Leu-NH₂, | |
| B-a-68: 3,4-[19]-Difluorobenzoyl-Ava-Gln-Trp-DAla-Val-βAla-His-Phe-Leu-NH₂, | |
| B-a-69: 3,4-[19]-Difluorobenzoyl-Ava-Gln-Trp-Ala-DVal-βAla-His-Phe-Leu-NH₂, | |
| B-a-70: 3,4-[19]-Difluorobenzoyl-Ava-Gln-Trp-Ala-Val-βAla-His-DPhe-Leu-NH₂, | |
| B-a-71: 3,4-[19]-Difluorobenzoyl-Ava-Gln-Trp-Ala-Val-βAla-His-βhIle-tbuGly-NH₂, | (SEQ ID NO: 212) |
| B-a-72: 4-[19]-Fluoro-3-cyano-phenylsulfonyl-Ava-Gln-Trp-Ala-Val-NMeGly-His-4- | (SEQ ID NO: 213) |
| Am,5-MeHpA-Cpa-NH₂, | |
| B-a-73: 4-[19]-Fluoro-3-cyano-phenylsulfonyl-Ava-Gln-Trp-Ala-Val-NMeGly-His-Sta- | (SEQ ID NO: 214) |
| Cpa-NH₂, | |
| B-a-74: 4-[19]-Fluoro-3-cyano-phenylsulfonyl-Ava-Gln-Trp-Ala-Val-NMeGly-His-Sta- | (SEQ ID NO: 215) |
| tbuAla-NH₂, | |
| B-a-75: 4-[19]-Fluoro-3-cyano-phenylsulfonyl-Ava-Gln-Trp-Ala-Val-NMeGly-His-4- | (SEQ ID NO: 216) |
| Am,5-MeHpA-tbuAla-NH₂. | |

29. Compound according to any one of claims 1-26, comprising

| | |
|---|---|
| IIA-a-76: 4-[18]Fluoro-3-cyano-benzoyl-Ava-□-c[Lys-(NMe)Phe-1Nal-D-Trp-Lys- | 68 |
| Thr], | |
| IIA-a-77: 4-[18]Fluoro-3-cyano-benzoyl-Ava-□-c[Dpr-Met-(NMe)Phe-Tyr-D-Trp-Lys], | 62 |
| IIB-a-76: 4-[19]Fluoro-3-cyano-benzoyl-Ava-□-c[Lys-(NMe)Phe-1Nal-D-Trp-Lys- | 68 |
| Thr], | |
| IIB-a-77: 4-[19]Fluoro-3-cyano-benzoyl-Ava-□-c[Dpr-Met-(NMe)Phe-Tyr-D-Trp-Lys], | 62 |
| IIA-a-78: 4-[18]Fluoro-3-cyano-benzoyl-Ava-DCys-Leu-Ile-Thr-Arg-Cys-Arg-Tyr-NH₂ | |
| IIA-a-79: 4-[18]Fluoro-3-cyano-benzoyl-Ava-DCys-Leu-Ile-Val-Arg-Cys-Arg-Tyr-NH₂ | |
| IIA-a-78: 4-[19]Fluoro-3-cyano-benzoyl-Ava-DCys-Leu-Ile-Thr-Arg-Cys-Arg-Tyr-NH₂, | |
| IIA-a-79: 4-[19]Fluoro-3-cyano-benzoyl-Ava-DCys-Leu-Ile-Val-Arg-Cys-Arg-Tyr- | |
| NH₂, | |
| 4-[19]Fluoro-3-cyano-benzoyl-(piperidyl-4-carbonyl)-Gln-Trp-Ala-Val-Gly-His(3Me)- | (SEQ ID NO: 217) |
| Sta-Leu-NH₂, | |
| 4-[19]Fluoro-3-cyano-benzoyl-(piperazin-1-yl-acetyl)-Gln-Trp-Ala-Val-Gly-His(3Me)-Sta- | (SEQ ID NO: 218) |
| Leu-NH₂, | |
| 4-[19]Fluoro-3-cyano-benzoyl-1,4-trans-Achc-Gln-Trp-Ala-Val-NMeGly-His-Sta-Leu- | (SEQ ID NO: 219) |
| NH₂, | |
| 3-cyano-4-fluoro-benzoyl-valyl-β-alanyl-phenylalanyl-glycine amide | (SEQ ID NO: 220) |
| [¹⁹F], | |
| 3-cyano-4-fluoro-benzoyl-valyl-β-alanyl-phenylalanyl-glycine amide | (SEQ ID NO: 221) |
| [¹⁸F], | |
| 3-cyano-4-fluoro-benzoyl-valyl-β-alanyl-histidyl(π-Me)-glycine amide | (SEQ ID NO: 222) |
| [¹⁹F], | |
| 3-cyano-4-fluoro-benzoyl-valyl-β-alanyl-histidyl(π-Me)-glycine amide | (SEQ ID NO: 223) |
| [¹⁸F], | |

3-cyano-4-fluoro-benzoyl-(5-aminopentanoyl)-phenylalanyl-(4(S)-amino-3(S)-hydroxy-6-methyl)heptanoyl-leucine amide [¹⁹F],
3-cyano-4-fluoro-benzoyl-(5-aminopentanoyl)-phenylalanyl-(4(S)-amino-3(S)-hydroxy-6-methyl)heptanoyl-leucine amide [¹⁸F],
3-Cyano-4-[F-19]fluoro-N-(thymidinyl-propyl)-benzamide,
3-Cyano-4-[F-18]fluoro-N-(thymidinyl-propyl)-benzamide,
3-Cyano-4-[F-19]fluoro-N-(2-[2-thymidinyl-ethoxy]-ethyl)-benzamide,
3-Cyano-4-[F-18]fluoro-N-(2-[2-thymidinyl-ethoxy]-ethyl)-benzamide,
3-Cyano-4-[F-19]fluoro-N-(thymidinyl-hexyl)-benzamide,
3-Cyano-4-[F-18]fluoro-N-(thymidinyl-hexyl)-benzamide,
3-Cyano-4-[19F]fluoro-N-(thymidinyl-butyl)benzamide,
3-Cyano-4-[19F]fluoro-N-(thymidinyl-butyl)benzamide,
3-Cyano-4-fluoro-N-(trifluoromethyl thymidinyl-hexyl)benzamide,
3-Cyano-4-fluoro-N-(trifluoromethyl thymidinyl-hexyl)benzamide,
3-Cyano-4-fluoro[F-18]-N-{6-[3-((2R,4S,5R)-4-hydroxy-5-hydroxymethyl-tetrahydro-thiophen-2-yl)-5-methyl-2,6,dioxo-3,6-dihydro-2H-pyrimidin-1-yl]-hexyl}-benzamide,
3-Cyano-4-fluoro[F-19]-N-{6-[3-((2R,4S,5R)-4-hydroxy-5-hydroxymethyl-tetrahydro-thiophen-2-yl)-5-methyl-2,6,dioxo-3,6-dihydro-2H-pyrimidin-1-yl]-hexyl}-benzamide. 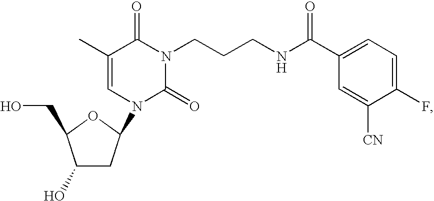
3-Cyano-4-[F-19]fluoro-N-(thymidinyl-propyl)-benzamide,
3-Cyano-4-[F-18]fluoro-N-(thymidinyl-propyl)-benzamide; 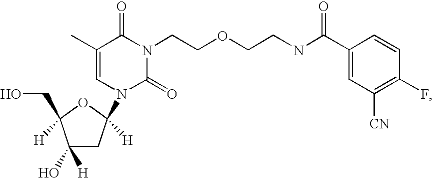
3-Cyano-4-[F-19]fluoro-N-(2-[2-thymidinyl-ethoxy]-ethyl)-benzamide,
3-Cyano-4-[F-18]fluoro-N-(2-[2-thymidinyl-ethoxy]-ethyl)-benzamide; 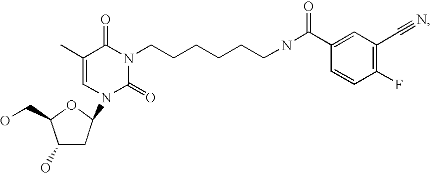
3-Cyano-4-[F-19]fluoro-N-(thymidinyl-hexyl)-benzamide,
3-Cyano-4-[F-18]fluoro-N-(thymidinyl-hexyl)-benzamide; 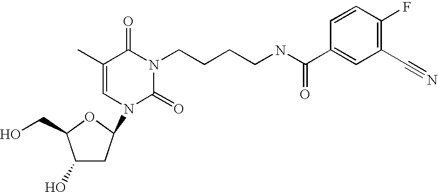
3-Cyano-4-[19F]fluoro-N-(thymidinyl-butyl)benzamide,
3-Cyano-4-[18F]fluoro-N-(thymidinyl-butyl)benzamide; 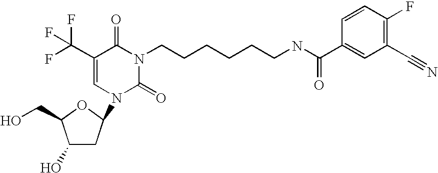
wherein F is ¹⁸F or ¹⁹F,
3-Cyano-4-fluoro-N-(trifluoromethyl thymidinyl-hexyl)benzamide,
3-Cyano-4-fluoro-N-(trifluoromethyl thymidinyl-hexyl)benzamide; 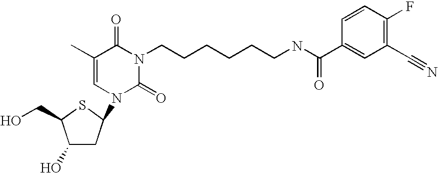
wherein F is ¹⁸F or ¹⁹F,
3-Cyano-4-fluoro[F-18]-N-{6-[3-((2R,4S,5R)-4-hydroxy-5-hydroxymethyl-tetrahydro-thiophen-2-yl)-5-methyl-2,6,dioxo-3,6-dihydro-2H-pyrimidin-1-yl]-hexyl}-benzamide;
3-Cyano-4-fluoro[F-19]-N-{6-[3-((2R,4S,5R)-4-hydroxy-5-hydroxymethyl-tetrahydro-thiophen-2-yl)-5-methyl-2,6,dioxo-3,6-dihydro-2H-pyrimidin-1-yl]-hexyl}-benzamide;

| | |
|---|---|
| 3-CN,4-F-Bz-Ava-Gln-Trp-Ala-Val-Gly-His-FA01010-Leu-NH2, | (SEQ ID NO: 224) |
| 4F,3CN-Bnz-Arg-Ava-Gln-Trp-Ala-Val-NMeGly-His(3Me)-Sta-Leu-NH2, | (SEQ ID NO: 225) |
| 3-CF3,4-F-Benzoyl-Arg-Ava-Gln-Trp-Ala-Val-NMeGly-His-Sta-Leu-NH2, | (SEQ ID NO: 226) |
| 3-CN,4-F-Benzoyl-Arg-Ava-Gln-Trp-Ala-Val-Gly-His(3Me)-Sta-Leu-NH2, | (SEQ ID NO: 227) |
| 3-CN,4-F-Benzoyl-Arg-Ava-Gln-Trp-Ala-Val-NMeGly-His-Sta-Leu-NH2, | (SEQ ID NO: 228) |

wherein F is ¹⁸F or ¹⁹F.

30. The compound according to claims 1-26, wherein P is selected from the group comprising

| | |
|---|---|
| SEQ ID NO: 1 | Gln-Trp-Ala-Val-NMeGly-His-Sta-Leu-NH₂ |
| SEQ ID NO: 2 | Gln-Trp-Ala-Val-Gly-His(Me)-Sta-Leu-NH₂ |
| SEQ ID NO: 3 | Gln-Trp-Ala-Val-NMeGly-His(3Me)-Sta-Leu-NH₂ |
| SEQ ID NO: 4 | Gln-Trp-Ala-Val-Gly-His(3Me)-Sta-Leu-NH₂ |
| SEQ ID NO: 7 | Gln-Trp-Ala-Val-NMeGly-His(3Me)-Sta-Cpa-NH₂ |
| SEQ ID NO: 8 | Gln-Trp-Ala-Val-Gly-His(3Me)-4-Am,5-MeHpA-Leu-NH₂ |
| SEQ ID NO: 12 | Gln-Trp-Ala-Val-Gly-His(3Me)-4-Am,5-MeHpA-Leu-NH₂ |
| SEQ ID NO: 17 | Gln-Trp-Ala-Val-Gly-His-4-Am,5-MeHpA-Leu-NH₂ |
| SEQ ID NO: 23 | Gln-Trp-Ala-Val-NMeGly-His(3Me)-4-Am,5-MeHpA-Cpa-NH₂ |
| SEQ ID NO: 27 | Gln-Trp-Ala-Val-NMeGly-His-FA02010-Cpa-NH₂ |
| SEQ ID NO: 28 | Gln-Trp-Ala-Val-NMeGly-His-4-Am,5-MeHpA-tbuGly-NH₂ |
| SEQ ID NO: 30 | Gln-Trp-Ala-Val-NMeGly-His(3Me)-Sta-tBuGly-NH₂ |
| SEQ ID NO: 32 | Gln-Trp-Ala-Val-NMeGly-His(3Me)-4-Am,5-MeHpA-Leu-NH₂ |
| SEQ ID NO: 33 | Gln-DTrp-Ala-Val-Gly-His-4-Am,5-MeHpA-tbuGly-NH₂ |
| SEQ ID NO: 34 | Gln-DTrp-Ala-Val-Gly-His-4-Am-5-MeHxA-Cpa-NH₂ |
| SEQ ID NO: 35 | Gln-Trp-Ala-Val-NMeGly-His(3Me)-Sta-Cpa-NH₂ |
| SEQ ID NO: 36 | Gln-DTrp-Ala-Val-Gly-His-Sta-tbuAla-NH₂ |
| SEQ ID NO: 42 | Gln-Trp-Ala-Val-Gly-His(3Me)-Sta-Cpa-NH₂ |
| SEQ ID NO: 43 | Gln-Trp-Ala-Val-Gly-His(3Me)-Sta-tBuGly-NH₂ |
| SEQ ID NO: 46 | Gln-Trp-Ala-Val-Gly-His(3Me)-4-Am,5-MeHpA-Leu-NH₂ |
| SEQ ID NO: 48 | Gln-Trp-Ala-Val-Gly-His(3Me)-4-Am,5-MeHpA-Leu-NH₂ |
| SEQ ID NO: 5 | Gln-Trp-Ala-Val-Gly-NMeHis-4-Am,5-MeHpA-Cpa-NH₂ |
| SEQ ID NO: 49 | Gln-Trp-Ala-Val-Gly-NMeHis(3Me)-4-Am,5-MeHpA-Leu-NH₂ |
| SEQ ID NO: 50 | Gln-Trp-Ala-Val-Gly-NMeHis-4-Am,5-MeHpA-Leu-NH₂ |
| SEQ ID NO: 51 | Gln-Trp-Ala-Val-NMeGly-Hls-AHMHxA-Leu-NH₂ |
| SEQ ID NO: 52 | Gln-Trp-Ala-Val-βAla-NMeHis-Tha-Cpa-NH₂ |
| SEQ ID NO: 53 | Gln-Trp-Ala-Val-βAla-NMeHis-Phe-Cpa-NH₂ |
| SEQ ID NO: 54 | Gln-Trp-Ala-Val-βAla-NMeHis-Phe-Leu-NH₂ |
| SEQ ID NO: 55 | Gln-Trp-Ala-Val-βAla-DHis-Phe-Leu-NH₂ |
| SEQ ID NO: 56 | Gln-Trp-Ala-Val-βAla-His-βhLeu-Leu-NH₂ |
| SEQ ID NO: 57 | Gln-Trp-Ala-Val-βAla-His-βhIle-Leu-NH₂ |
| SEQ ID NO: 58 | Gln-Trp-Ala-Val-βAla-His-βhLeu-tbuGly-NH₂ |
| SEQ ID NO: 59 | Gln-Trp-Ala-Val-βAla-His(3Me)-Phe-Tha-NH₂ |
| SEQ ID NO: 60 | Gln-Trp-Ala-Val-βAla-His(3Me)-Phe-Nle-NH₂ |
| SEQ ID NO: 61 | Gln-Trp-Ala-Val-βAla-NMeHis-Phe-tbuGly-NH₂ |
| SEQ ID NO: 62 | Gln-Trp-Ala-Val-βAla-NMeHis-Tha-tbuGly-NH₂ |
| SEQ ID NO: 63 | Gln-Trp-Ala-Val-βAla-His(3Me)-Tha-tbuGly-NH₂ |
| SEQ ID NO: 64 | Gln-Trp-Ala-Val-βAla-His(3Me)-Phe-Cpa-NH₂ |
| SEQ ID NO: 65 | Gln-Trp-Ala-NMeVal-βAla-His-Phe-Leu-NH₂ |
| SEQ ID NO: 66 | Gln-Trp-Ala-Val-βAla-His-NMePhe-Leu-NH₂ |
| SEQ ID NO: 67 | Gln-DTrp-Ala-Val-βAla-His-Phe-Leu-NH₂ |
| SEQ ID NO: 68 | Gln-Trp-DAla-Val-βAla-His-Phe-Leu-NH₂ |
| SEQ ID NO: 69 | Gln-Trp-Ala-DVal-βAla-His-Phe-Leu-NH₂ |
| SEQ ID NO: 70 | Gln-Trp-Ala-Val-βAla-His-DPhe-Leu-NH₂ |
| SEQ ID NO: 71 | Gln-Trp-Ala-Val-βAla-His-βhIle-tbuGly-NH₂ |
| SEQ ID NO: 72 | Gln-Trp-Ala-Val-NMeGly-His-4-Am,5-MeHpA-Cpa-NH₂ |
| SEQ ID NO: 73 | Gln-Trp-Ala-Val-NMeGly-His-Sta-Cpa-NH₂ |
| SEQ ID NO: 74 | Gln-Trp-Ala-Val-NMeGly-His-Sta-tbuAla-NH₂ |
| SEQ ID NO: 75 | Gln-Trp-Ala-Val-NMeGly-His-4-Am,5-MeHpA-tbuAla-NH₂ |
| SEQ ID NO: 77 | Gln-Trp-Ala-Val-His(Me)-Sta-Leu-NH₂ |
| SEQ ID NO: 82 | Gln-Trp-Ala-Val-Gly-His(3Me)-FA4-Am,5-MeHpA-Leu-NH₂ |
| SEQ ID NO: 90 | Gln-Trp-Ala-Val-Gly-His(3Me)-4-Am,5-MeHpA-Leu-NH₂ |
| SEQ ID NO: 91 | Gln-Trp-Ala-Val-Gly-His-4-Am,5-MeHpA-Leu-NH₂ |
| SEQ ID NO: 101 | Gln-Trp-Ala-Val-Gly-His(3Me)-4-Am-5-MeHpA-4- |
| | amino-5-methylheptanoic acid-Leu-NH₂ |
| SEQ ID NO: 102 | Gln-Trp-Ala-Val-NMeGly-His(3Me)-4-Am-5-MeHpA-4- |
| | amino-5-methylheptanoic acid-Cpa-NH_{2.} |

31. Method of preparing a compound having general chemical Formula II, wherein K=W, according to any one of claims 1-30, in which method a compound having general chemical Formula A, wherein K=LG-O, is labelled with fluorine isotope.

32. Method according to claim 31, comprising the step of coupling a compound having general chemical Formula A, wherein K=LG-O, according to any one of claims 1-30, with fluorine isotope to form a compound having general chemical Formula II, wherein K=W, or a pharmaceutically acceptable salt, hydrate or solvate thereof.

33. Method according to claims 31 and 32 wherein W is fluorine isotope and more preferably ¹⁸F.

34. A composition comprising a compound having general chemical Formula A, wherein K=LG-O or W, according to any one of claims 1-30, and a pharmaceutically acceptable carrier, diluent, adjuvant or excipient.

35. A method for imaging diseases, the method comprising introducing into a patient a detectable quantity of a labelled compound having general chemical Formula A, wherein K=W, according to any one of claims 1-30, or of a pharmaceutically acceptable salt, hydrate, ester, amide, solvate and prodrug thereof.

36. A method according to claim 35 wherein W is ¹⁸F.

37. A kit comprising a sealed vial containing a predetermined quantity of a compound having general chemical Formula A, wherein K=LG-O, according to any one of claims 1-30, or a pharmaceutically acceptable salt, hydrate, ester, amide, solvate and prodrug thereof.

38. A compound having general chemical Formula A, wherein K=LG-O or W, according to any one of claims 1-30, or a pharmaceutically acceptable salt, hydrate, ester, amide, solvate and prodrug thereof for use as medicament.

39. A compound having general chemical Formula A, wherein K=W, according to any one of claims 1-30, or a pharmaceutically acceptable salt, hydrate, ester, amide, solvate and prodrug thereof for use as diagnostic imaging agent.

40. A compound having general chemical Formula A, wherein K=W, according to any one of claims 1-30, or a pharmaceutically acceptable salt, hydrate, ester, amide, solvate and prodrug thereof for use as imaging agent for positron emission tomography (PET).

41. A compound according to claims 38 to 40 wherein W is fluorine isotope and more preferably ¹⁸F.

42. Use of a compound having general chemical Formula A, wherein K=LG-O or W, according to any one of claims 1-30, or of a pharmaceutically acceptable salt, hydrate, ester, amide, solvate and prodrug thereof for the manufacture of a medicament.

43. Use of a compound having general chemical Formula A, wherein K=LG-O or W, according to any one of claims 1-30, or of a pharmaceutically acceptable salt, hydrate, ester, amide, solvate and prodrug thereof for the manufacture of a diagnostic imaging agent.

44. The use according to claim 43 for the manufacture of a diagnostic imaging agent for imaging tissue at a target site using the imaging agent.

45. The use according to claim 44 wherein the imaging agent is positron emission tomography (PET) imaging agent.

46. A compound having general chemical Formula V: 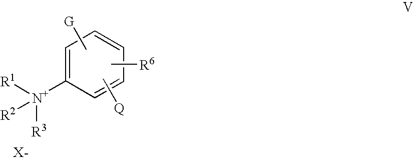
wherein N⁺(R¹)(R²)(R³), X⁻, -G, and -Q, have the same meaning as depicted above for compounds having general chemical Formula A and
R⁶ is selected from the group comprising —S(O)₂—N(H)—CH₂—C(O)OH, —S(O)₂—N(Me)—CH₂—C(O)OH and C(O)OH.

47. A method of preparing compound of Formula A wherein K=LG-O by reacting a compound of Formula V with a targeting agent.

48. The method according to claim 47 wherein the compound of Formula A wherein K=LG-O and the targeting agent are reacted optionally with a condensing agent.

49. Peptide sequence selected from

| | |
|---|---|
| SEQ ID NO: 1 | Gln-Trp-Ala-Val-NMeGly-His-Sta-Leu-NH₂ |
| SEQ ID NO: 2 | Gln-Trp-Ala-Val-Gly-His(Me)-Sta-Leu-NH₂ |
| SEQ ID NO: 3 | Gln-Trp-Ala-Val-NMeGly-His(3Me)-Sta-Leu-NH₂ |
| SEQ ID NO: 4 | Gln-Trp-Ala-Val-Gly-His(3Me)-Sta-Leu-NH₂ |
| SEQ ID NO: 7 | Gln-Trp-Ala-Val-NMeGly-His(3Me)-Sta-Cpa-NH₂ |
| SEQ ID NO: 8 | Gln-Trp-Ala-Val-Gly-His(3Me)-4-Am,5-MeHpA-Leu-NH₂ |
| SEQ ID NO: 12 | Gln-Trp-Ala-Val-Gly-His(3Me)-4-Am,5-MeHpA-Leu-NH₂ |
| SEQ ID NO: 17 | Gln-Trp-Ala-Val-Gly-His-4-Am,5-MeHpA-Leu-NH₂ |
| SEQ ID NO: 23 | Gln-Trp-Ala-Val-NMeGly-His(3Me)-4-Am,5-MeHpA-Cpa-NH₂ |
| SEQ ID NO: 27 | Gln-Trp-Ala-Val-NMeGly-His-FA02010-Cpa-NH₂ |
| SEQ ID NO: 28 | Gln-Trp-Ala-Val-NMeGly-His-4-Am,5-MeHpA-tbuGly-NH₂ |
| SEQ ID NO: 30 | Gln-Trp-Ala-Val-NMeGly-His(3Me)-Sta-tBuGly-NH₂ |
| SEQ ID NO: 32 | Gln-Trp-Ala-Val-NMeGly-His(3Me)-4-Am,5-MeHpA-Leu-NH₂ |
| SEQ ID NO: 33 | Gln-DTrp-Ala-Val-Gly-His-4-Am,5-MeHpA-tbuGly-NH₂ |
| SEQ ID NO: 34 | Gln-DTrp-Ala-Val-Gly-His-4-Am-5-MeHxA-Cpa-NH₂ |
| SEQ ID NO: 35 | Gln-Trp-Ala-Val-NMeGly-His(3Me)-Sta-Cpa-NH₂ |
| SEQ ID NO: 36 | Gln-DTrp-Ala-Val-Gly-His-Sta-tbuAla-NH₂ |
| SEQ ID NO: 42 | Gln-Trp-Ala-Val-Gly-His(3Me)-Sta-Cpa-NH₂ |
| SEQ ID NO: 43 | Gln-Trp-Ala-Val-Gly-His(3Me)-Sta-tBuGly-NH₂ |
| SEQ ID NO: 46 | Gln-Trp-Ala-Val-Gly-His(3Me)-4-Am,5-MeHpA-Leu-NH₂ |
| SEQ ID NO: 48 | Gln-Trp-Ala-Val-Gly-His(3Me)-4-Am,5-MeHpA-Leu-NH₂ |
| SEQ ID NO: 5 | Gln-Trp-Ala-Val-Gly-NMeHis-4-Am,5-MeHpA-Cpa-NH₂ |
| SEQ ID NO: 49 | Gln-Trp-Ala-Val-Gly-NMeHis(3Me)-4-Am,5-MeHpA-Leu-NH₂ |
| SEQ ID NO: 50 | Gln-Trp-Ala-Val-Gly-NMeHis-4-Am,5-MeHpA-Leu-NH₂ |
| SEQ ID NO: 51 | Gln-Trp-Ala-Val-NMeGly-Hls-AHMHxA-Leu-NH₂ |
| SEQ ID NO: 52 | Gln-Trp-Ala-Val-βAla-NMeHis-Tha-Cpa-NH₂ |
| SEQ ID NO: 53 | Gln-Trp-Ala-Val-βAla-NMeHis-Phe-Cpa-NH₂ |
| SEQ ID NO: 54 | Gln-Trp-Ala-Val-βAla-NMeHis-Phe-Leu-NH₂ |
| SEQ ID NO: 55 | Gln-Trp-Ala-Val-βAla-DHis-Phe-Leu-NH₂ |
| SEQ ID NO: 56 | Gln-Trp-Ala-Val-βAla-His-βhLeu-Leu-NH₂ |
| SEQ ID NO: 57 | Gln-Trp-Ala-Val-βAla-His-βhIle-Leu-NH₂ |
| SEQ ID NO: 58 | Gln-Trp-Ala-Val-βAla-His-βhLeu-tbuGly-NH₂ |
| SEQ ID NO: 59 | Gln-Trp-Ala-Val-βAla-His(3Me)-Phe-Tha-NH₂ |
| SEQ ID NO: 60 | Gln-Trp-Ala-Val-βAla-His(3Me)-Phe-Nle-NH₂ |
| SEQ ID NO: 61 | Gln-Trp-Ala-Val-βAla-NMeHis-Phe-tbuGly-NH₂ |
| SEQ ID NO: 62 | Gln-Trp-Ala-Val-βAla-NMeHis-Tha-tbuGly-NH₂ |
| SEQ ID NO: 63 | Gln-Trp-Ala-Val-βAla-His(3Me)-Tha-tbuGly-NH₂ |
| SEQ ID NO: 64 | Gln-Trp-Ala-Val-βAla-His(3Me)-Phe-Cpa-NH₂ |
| SEQ ID NO: 65 | Gln-Trp-Ala-NMeVal-βAla-His-Phe-Leu-NH₂ |
| SEQ ID NO: 66 | Gln-Trp-Ala-Val-βAla-His-NMePhe-Leu-NH₂ |
| SEQ ID NO: 67 | Gln-DTrp-Ala-Val-βAla-His-Phe-Leu-NH₂ |
| SEQ ID NO: 68 | Gln-Trp-DAla-Val-βAla-His-Phe-Leu-NH₂ |
| SEQ ID NO: 69 | Gln-Trp-Ala-DVal-βAla-His-Phe-Leu-NH₂ |
| SEQ ID NO: 70 | Gln-Trp-Ala-Val-βAla-His-DPhe-Leu-NH₂ |
| SEQ ID NO: 71 | Gln-Trp-Ala-Val-βAla-His-βhIle-tbuGly-NH₂ |
| SEQ ID NO: 72 | Gln-Trp-Ala-Val-NMeGly-His-4-Am,5-MeHpA-Cpa-NH₂ |
| SEQ ID NO: 73 | Gln-Trp-Ala-Val-NMeGly-His-Sta-Cpa-NH₂ |
| SEQ ID NO: 74 | Gln-Trp-Ala-Val-NMeGly-His-Sta-tbuAla-NH₂ |
| SEQ ID NO: 75 | Gln-Trp-Ala-Val-NMeGly-His-4-Am,5-MeHpA-tbuAla-NH₂ |
| SEQ ID NO: 77 | Gln-Trp-Ala-Val-His(Me)-Sta-Leu-NH₂ |
| SEQ ID NO: 82 | Gln-Trp-Ala-Val-Gly-His(3Me)-FA4-Am,5-MeHpA-Leu-NH₂ |
| SEQ ID NO: 90 | Gln-Trp-Ala-Val-Gly-His(3Me)-4-Am,5-MeHpA-Leu-NH₂ |
| SEQ ID NO: 91 | Gln-Trp-Ala-Val-Gly-His-4-Am,5-MeHpA-Leu-NH₂ |
| SEQ ID NO: 101 | Gln-Trp-Ala-Val-Gly-His(3Me)-4-Am-5-MeHpA-4-amino-5- |
| | methylheptanoic acid-Leu-NH₂ |
| SEQ ID NO: 102 | Gln-Trp-Ala-Val-NMeGly-His(3Me)-4-Am-5-MeHpA-4-amino- |
| | 5-methylheptanoic acid-Cpa-NH_{2.} |
